(19) United States
(12) Patent Application Publication
(10) Pub. No.: US 2022/0287949 A1
(43) Pub. Date: Sep. 15, 2022

(54) SAFE PARTICLES FOR THE INTRODUCTION OF USEFUL CHEMICAL AGENTS IN THE BODY WITH CONTROLLED ACTIVATION

(71) Applicant: Bambu Vault LLC, Lowell, MA (US)

(72) Inventors: Glenn HORNER, West Roxbury, MA (US); Prakash RAI, Lowell, MA (US); Satish AGRAWAL, Sudbury, MA (US); Bethany PARKER, New Ipswich, NH (US); Joshna CHITTIGORI, Lowell, MA (US)

(73) Assignee: Bambu Vault LLC, Lowell, MA (US)

(21) Appl. No.: 17/432,902

(22) PCT Filed: Feb. 21, 2020
(86) PCT No.: PCT/US2020/019346

§ 371 Date: Aug. 20, 2021

Related U.S. Application Data
(60) Provisional application No. 62/808,724, filed on Feb. 21, 2019.

(51) Int. Cl. A61K 8/81; A61K 8/895; A61K 8/02; A61Q 1/02

(52) U.S. Cl. CPC A61K 8/8152; A61K 8/895; A61K 8/0241; A61Q 1/025; A61K 2800/43; A61K 2800/654; A61K 2800/81

(57) ABSTRACT

This invention provides particles encapsulating colorant that do not produce functional effects or remove functional effects until they are triggered by contacting with at least one exogenous source. The particles in this invention minimize toxic effects the body of the colorant and the material that interacts with an exogenous source as well as minimize body chemicals from degrading both the colorant and the material that interacts with an exogenous source inside the particle.

### CROSS-REFERENCE TO RELATED APPLICATIONS

This application claims priority to U.S. Provisional Patent Application No. 62/808,724, filed on Feb. 21, 2019, which is hereby incorporated by reference in its entirety.

### BACKGROUND OF THE INVENTION

Active agents, such as cosmetic agents (e.g., tattoo pigments) or material interacting with the exogenous source (e.g., dye excitable by the near infrared radiation), have many utilities in cosmetic, biomedical and pharmaceutical applications. The active agent and the material interacting with the exogenous source are typically organic compounds that can be susceptible to degradation by the body chemicals present in the bodily fluids. On the other hand, the active agent and the material may leach out, and cause cytotoxicity to the human body. For example, tattoo ink particles currently in the market contain a wide range of chemicals and heavy metals. Whereas these materials are not generally encapsulated, even after encapsulation, leaked agent toxicity can be very high unless the particle structure is modified to create a structure that significantly lowers the concentration outside the particle. These chemicals and heavy metals are potentially toxic. Recent research on the deceased tattooed individuals showed that the organic pigments, including phthalocyanines and azo compounds, are present in the skin and lymph samples of the tattooed individuals (Sci. Rep. 2017, DOI: 10.1038/s41598-017-11721-z).

It is also important to note that even with encapsulation of active agents, the efficacy of such agents can be negatively impacted by physiological media that can enter the encapsulated particle. This, of course, is dependent upon the duration that the particle has to reside in the body.

In some cases, cosmetic agents such as tattoo pigments are designed to stay inside a human body for a prolonged period of time, and thus have greater potential to be degraded over time by the various physiological media in the human body. Even with small leakage of physiological media, even with low particle porosity, significant degradation could occur over longer periods of time.

The reported encapsulation techniques generate particles in general that have some degree of porosity which allows chemical agents to escape out as well as allows the incursion of the bodily fluids into the particles in a time-dependent fashion.

Thus, there exists a need to create particles with controlled porosity to not only reduce toxicity from leakage of active agent outside the particle but also the loss of efficacy from breakdown of the agent due to the incursion of body chemicals into the particle.

### SUMMARY OF THE INVENTION

The disclosure provides particles comprising a colorant and a material that interacts with an exogenous source. Such particles minimize toxic effects of the colorant and the material to the body as well as minimize body chemicals from degrading both the colorant and the material inside the particle. In one embodiment, the colorant does not exhibit any functional effects until activated by an exogenous source.

In an embodiment, this disclosure provides a particle comprising a carrier, a material, and a colorant, wherein the color of the colorant changes when the material absorbs radiation at infrared wavelengths; wherein the colorant and the material in the particle exhibit stability such that the particle is considered passing the Efficacy Determination Protocol; and wherein the particle structure is constructed such that it passes the Extractable Cytotoxicity Test.

In some embodiments, the color is changed to colorless. In some embodiments, the color is changed from one hue to a different hue.

In some embodiments, the carrier comprises a polymer or copolymer of methylmethacrylate.

In some embodiments, the infrared wavelengths of the radiation are in the range of 700 to 1500 nm. In some embodiments, the infrared wavelength of the radiation is at 1064 nm.

In some embodiments, the material for absorbing the radiation at infrared wavelengths is a tetrakis aminium dye. In some embodiments, the material for absorbing the radiation at infrared wavelengths is a zinc iron phosphate pigment.

In some embodiments, the colorant comprises a chromophore group and a thermally activatable fragmentation group. In some embodiments, the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof. In some embodiments, the thermally activatable fragmentation group produces a nucleophilic group upon activation. In some embodiments, the thermally activatable fragmentation group comprises substituted and unsubstituted carbonates, carbamates, esters, lactams, lactones, amides, imides, oximes, sulfonates, or phosphonates.

In some embodiments, the particle is amorphous, partially amorphous, or partially crystalline.

In some embodiments, the particle further comprises a shell enclosing the particle to form a core-shell particle. In some embodiments, the shell comprises a cross-linked polymer. In some embodiments, the shell comprises an organosilicate polymer derived from vinyl trimethoxysilane reagent in Stober synthesis.

In an embodiment, the disclosure provides a particle comprising: (a) a core comprising a carrier, a material, and a colorant, (b) a shell enclosing the core, wherein the material absorbs radiation at infrared wavelengths, wherein the colorant is transformed to colorless when the material absorbs radiation at infrared wavelengths, wherein the colorant and the material in the particle exhibit stability such that the particle is considered passing the Efficacy Determination Protocol; and further wherein the particle structure is constructed such that it passes the Extractable Cytotoxicity Test.

In an embodiment, the particle passes the Extractable Cytotoxicity Test at the extract concentration.

In an embodiment, the particle passes the Extractable Cytotoxicity Test up to 0.1× dilution of the extract concentration.

In an embodiment, the particle passes the Extractable Cytotoxicity Test up to 0.01× dilution of the extract concentration.

In an embodiment, the particle passes the Extractable Cytotoxicity Test up to 0.001× dilution of the extract concentration.

In an embodiment, the particle passes the Extractable Cytotoxicity Test up to 0.0001× dilution of the extract concentration.

In some embodiments, the carrier comprises a polymer or copolymer of methylmethacrylate.

In some embodiments, the shell is a cross-linked polymer. In some embodiments, the shell comprises a silicate polymer derived from vinyl trimethoxysilane.

In some embodiments, the infrared wavelengths of the radiation are in the range of 700 to 1500 nm. In some embodiments, the infrared wavelength of the radiation is at 1064 nm.

In some embodiments, the material for absorbing the radiation at infrared wavelengths is a tetrakis aminium dye. In some embodiments, the material for absorbing the radiation at infrared wavelengths is a zinc iron phosphate pigment.

In some embodiments, the colorant comprises a chromophore group and a thermally activatable fragmentation group. In some embodiments, the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof. In some embodiments, the thermally activatable fragmentation group produces a nucleophilic group upon activation.

In some embodiments, the thermally activatable fragmentation group comprises substituted and unsubstituted carbonates, carbamates, esters, lactams, lactones, amides, imides, oximes, sulfonates, or phosphonates.

In an embodiment, this disclosure provides a particle comprising: (a) a core comprising a carrier, a material, and a thermally-activatable colorant; and (b) a shell enclosing the core; wherein the shell comprises a cross-linked organo-silicate polymer derived from trialkoxysilane, or trihalorosilane; wherein the thermally activatable colorant is transformed to colorless when the material absorbs radiation at infrared wavelengths and converts the energy to heat; wherein the thermally activatable colorant and the material in the particle exhibit stability such that the particle is considered passing the Efficacy Determination Protocol; and wherein the particle structure is constructed such that it passes the Extractable Cytotoxicity Test.

In some embodiments, the carrier comprises a polymer or copolymer of methylmethacrylate.

In some embodiments, the trialkoxysilane used for making the shell is selected from the group of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group of trichlorosilane, tribromosilane, triiodosilane, and combinations thereof. In some embodiments, the cross-linked organo-silicate polymer is derived from vinyl-trimethoxysilane (VTMS).

In some embodiments, the thermally activatable colorant comprises a chromophore group and a thermally activatable fragmentation group. In some embodiments, the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof. In some embodiments, the thermally activatable fragmentation group produces a nucleophilic group upon activation. In some embodiments, the thermally activatable fragmentation group comprises substituted and unsubstituted carbonates, carbamates, esters, lactams, lactones, amides, imides, oximes, sulfonates, or phosphonates.

In some embodiments, the infrared wavelengths of the radiation are in the range of 700 to 1500 nm. In some embodiments, the infrared wavelengths of the radiation is at 1064 nm.

In some embodiments, the material for absorbing radiation at infrared wavelength is a tetrakis aminium dye.

In an embodiment, this disclosure provides a tattoo ink for the permanent removable tattoo comprising the tattoo particles described herein and a dermatologically acceptable liquid carrier.

In some embodiments, the tattoo ink is in the form of an injectable suspension.

In some embodiments, the dermatologically acceptable liquid carrier is selected from the group of purified water, witch hazel, Listerine® mouthwash, and buffer solution. In some embodiments, the dermatologically acceptable liquid carrier comprises a buffer solution having pH ranging from about 6 to about 8. In some embodiments, the buffer solution is a hydrogen ion buffer selected from the buffers listed in Table A.

In an embodiment, this disclosure provides a method of making permanent removable tattoos in a subject comprising the step of injecting any one of the herein described tattoo ink to an area of skin of the subject to form a permanent tattoo on the subject.

In an embodiment, this disclosure provides a method of remotely triggering color-change from tattoo particles comprising the step of administering a dose of laser to the permanent tattoos on the subject as described herein.

In some embodiments, the method further comprises repeating the step of administering a dose of laser.

In some embodiments, the laser is a pulsed laser light. In some embodiments, the laser pulse duration is in a range from milliseconds to nanoseconds, and the laser has an oscillation wavelength at 1064 nm. In some embodiments, the laser emits light at 808 nm. In some embodiments, the laser emits light at 805 nm. In some embodiments, the carrier is crosslinked.

In some embodiments, the laser pulse duration is selected from the group of about 10 ns; about 400 ps to about 500 ps; about 500 ps to about 600 ps, and about 600 ps to about 750 ps. In some embodiments, the laser emits light at 1064 nm. In some embodiments, the material for absorbing the radiation at infrared wavelengths is Epolight IR 1117.

In some embodiments, the colorant is one or more selected from the group of magenta, cyan, yellow, black and PB5 disclosed in the Table 1. In some embodiments, the colorant is one or more selected from the group of magenta, cyan, yellow, black and PBS disclosed in the Table 3.

### BRIEF DESCRIPTION OF THE DRAWINGS

FIG. 1 illustrates the flowchart of the feedback loop for identifying optimal particle structure.

FIG. 2 illustrates a typical particle size distribution for 2 μm particles as measured with a Horiba LA-950 particle size analyzer in distilled water with pH 7.4.

FIG. 3 illustrates the absorbance spectra of the extract of the colorant leached into 3 mL 1% SDS from 3 μm PB1 particles with no VTMS shell, with a 9.1% VTMS shell, with a 25% VTMS shell, and with a 40% VTMS shell. For all particles, the core contains 3:1 weight ratio of polymer to dye.

FIG. 4A illustrates the absorbance spectrum of the extract of the colorant leached into 3 mL 1% SDS from the 1 μm, NB particles having a 25% VTMS shell, compared to 1 μm particles without a 25% VTMS shell, of which the core contains 3:1 weight ratio of polymer to dye. FIG. 4B illustrates the absorbance spectrum of the extract of the dye leached in 3 mL 1% SDS from the 0.5 μm PB4 particles having a 25% VTMS shell, compared to 0.5 μm particles without the shell, of which the core contains 2:1 weight ratio of polymer to dye. FIG. 4C illustrates the absorbance spectrum of the extract of the dye leached in 3 mL 1% SDS from 0.7 μm process black particles having a 25% VTMS shell, of which the core contains 3:1 weight ratio of polymer to the process black (PB) dye.

FIG. 5A illustrates the SEM image for 3 μm, NB particles having no shell, of which the core contains 3:1 weight ratio of polymer to dye. FIG. 5B illustrates the SEM image for 1 μm, NB particles having a 25% VTMS shell, of which the core contains 3:1 weight ratio of polymer to dye. FIG. 5C illustrates the TEM image for 0.7 μm PB1 dye particles having a 25% VTMS shell, of which the core contains 3:1 weight ratio of polymer to dye.

FIG. 6 illustrates the absorbance spectra of the extract of the dye leached in 3 mL 1% SDS from 0.7 μm, PB1 particles having a 25% TEOS shell as compared with uncoated particles, of which the core contains 3:1 weight ratio of polymer to dye.

FIG. 7 illustrates the absorbance spectra of the extract of the dye leached in 3 mL 1% SDS from 0.9 mm, PB1 particles having a 25% VTMS shell, and particles having a shell made from the VTMS/TEOS mixture, of which the core contains 3:1 weight ratio of polymer to dye.

FIG. 8 illustrates the absorption spectra of Epolight™ 1117 in methanol, and in neutrophil media after 0, 10, and 20 minutes of exposure.

FIG. 9 illustrates the absorption spectra of Epolight™ 1117 in methanol, and in macrophage media after 0 and 15 minutes of exposure.

FIG. 10 illustrates the absorbance spectra of the extract of the Y197 dye and Epolight™ 1117 leached in dichloromethane (DCM) after treatment with a dose of laser irradiation at 1064 nm wavelength and a fluence of 3.51 J/cm² as compared with the control (Y197 particle without laser treatment). After being treated with the laser, 68% Epolight™ 1117 and 41% of Y197 dye in the particle were degraded. The results indicated that Epolight™ 1117 had significant absorption of the laser and localized heat generation inside the particle to cause the degradation of Y197 dye.

FIG. 11 illustrates the absorbance spectra of the extract of the M071 dye and EpolightTM 1117 leached in DCM after treatment with a dose of laser irradiation at 1064 nm wavelength and a fluence at 2.46 J/cm², 3.03 J/cm², 3.51 J/cm², 4.28 J/cm², and 5.09 J/cm² as compared with the control (M071 particle without laser treatment). The results demonstrated that the decay of the IR absorbing agent in M071 particles is 80% vs a decay of about 50% for the magenta dye. Furthermore, the dye decay in M071 particles leveled off at 4.28 J/cm²

FIG. 12 illustrates the absorbance spectra of the extract of the PB5 dye and Epolight™ 1117 leached in DCM after treatment with a dose of laser irradiation at 1064 nm wavelength and a fluence at 3.51 J/cm², 4.28 J/cm², and 5.09 J/cm² as compared with the control (PB5 particle without laser treatment). The results demonstrated that low level of color clearing for 5% PBS particles at about 30%. Furthermore, the dye decay in 5% PB5 particles appears to level off at 3.51 J/cm². No additional heat was generated from the higher fluence which suggests that the IR absorbing agent absorbance is saturated at 3.51 J/cm².

### DETAILED DESCRIPTION OF THE INVENTION

Tattoos are a form of tissue marking that have been practiced for thousands of years. Tattoos today may be used for personal artistic expression, or may also be used for cosmetic modification, in particular on faces. Tattoos are prepared by introducing an ink prepared from pigments to the dermis of the skin; pigment that reaches only the epidermis can be sloughed off over time. Typical pigments include carbon black, inorganic metal salts, and organometallic complexes. Such ingredients are generally not regulated for tissue marking applications, and these materials have been known to cause biological responses, including allergic reaction, which can be severe even long after the original exposure.

Many people who obtain tattoos decide after a period of time that they would like to have them removed. Such “buyer's remorse” has led to an industry for tattoo removal using such methods as over-tattooing, dermabrasion, surgical excision, and removal by irradiation with pulsed lasers. All of these methods are quite invasive and can lead to additional skin trauma and scarring as well as to the possibilities of secondary infection (from surgical approaches) or collateral damage (from abrasive and laser methods).

Several methods to produce tissue marking that may be subsequently removed by non-invasive methods have been described. Anderson (US Pat. No. 6,800,122) discloses a tattoo ink particle comprising a core of at least one pigment or dye, an absorption component and a vehicle surrounding the core, wherein the absorption component alters the structure of the particles when exposed to an exogenous energy such that the dye is released from the particles and eventually digested by the human body. Agrawal (US Pat. No. 8,039,193) discloses a method of tissue marking that includes at least one colored compound comprising a thermally labile fragmentation group and at least one infrared absorbing compound such that the color of the dye in the tattoo pigment can be rendered colorless by the application of infrared radiation. Such methods do not consider the toxic nature of the components used. Particles comprising amorphous polymers contain inherent free volume or porosity that can be penetrated by body fluids, allowing the leakage of toxic components into the extracellular matrix. Likewise, such body fluids can carry with them molecular components that can attack the colorant inside the particles, rendering it loss of function. Furthermore, even inorganic materials can have inherent porous structures that provide pathways to incursion by biological agents.

For tissue marking application, it would be advantageous to provide particles having decreased toxicity of pigments, dyes and chromophores. It would be even more desirable to provide particles having more controlled color removal properties.

In an embodiment, the disclosure provides a particle suitable for tissue marking comprising: (a) a core comprising a carrier, a material, and a colorant, (b) a shell enclosing the core, wherein the material absorbs radiation at infrared wavelengths, wherein the colorant is transformed to a colorless dye when the material absorbs radiation at infrared wavelengths, wherein the colorant and the material in the particle exhibit stability such that the particle is considered passing the Efficacy Determination Protocol; and further wherein the particle structure is constructed such that it passes the Extractable Cytotoxicity Test.

Such particles minimize the toxic effects of any color dye and the material that interacts with the exogenous source which have leaked out of the particle into the body as well as minimize the entry of body chemicals inside the particle at concentrations that can degrade both the color dye and the material inside the particle.

The encapsulation of the colorant and/or the material with a polymer may reduce the degradation and the leakage mentioned above, but only to some extent due to the inherent porosity of the polymeric particle.

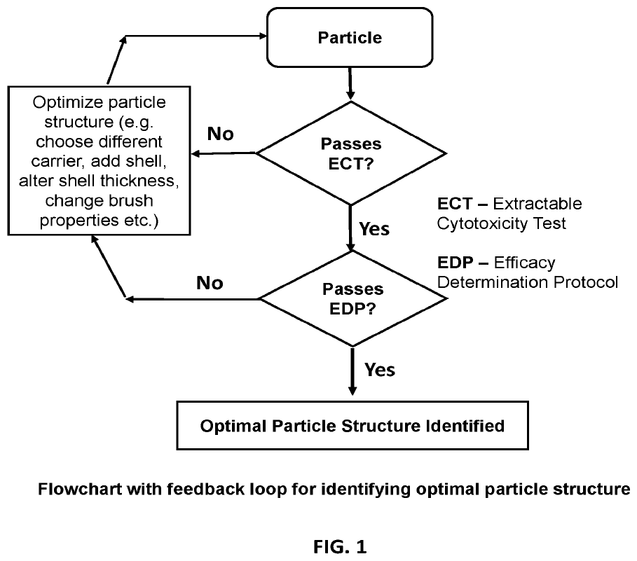

The porosity of a particle depends on various factors, including the molecular weight of the polymer, the structure of the polymer, the cross-linker and the amount thereof, the polymerization temperature, and solvent, etc. Further, when treating a disease with polymeric particles comprising a therapeutic agent, for example, the tolerable leakage of the therapeutic agent for any specific disease is different from that of another. Therefore, it is desirable to have an efficient method of controlling the particle porosity. To this end, present invention provides a process methodology to arrive at a solution to such problems. Specifically, the present invention provides a method of controlling porosity of the polymeric particles via a feedback loop depicted in FIG. 1, resulting in much safer particles for human use. As shown in FIG. 1, the particle structure is sequentially optimized to reduce: (1) the toxicity of agents and materials that leak out of the particle to healthy cells, and (2) the loss of efficacy of the agents and materials from breakdown due to the entry of body chemicals into the particle. To this end, the present invention provides a particle comprising:(a) a colorant, (b) a carrier, (c) a material that interacts with an exogenous source, wherein the colorant is encapsulated by the carrier, wherein the colorant and the material in the particle exhibit stability such that the particle is considered passing the Efficacy Determination Protocol; and wherein the particle structure is constructed such that it passes the Extractable Cytotoxicity Test. Furthermore, the particles described herein reduce the toxicity of the colorant.

### Definitions

As used in the preceding sections and throughout the rest of this specification, unless defined otherwise, all technical and scientific terms used herein have the same meaning as is commonly understood by one of skill in the art to which this invention belongs. All patents and publications referred to herein are incorporated by reference in their entireties.

The terms “a”, “an”, and “the” as used herein, generally is construed to cover both the singular and the plural forms.

The term “about” as used herein, generally refers to a particular numeric value that includes variation and an acceptable error range as determined by one of ordinary skill in the art, which will depend in part on how the numeric value is measured or determined, i.e., the limitations of the measurement system. For example, “about” can mean zero variation, and a range of ±20%, ±10%, or ±5% of a given numeric value.

The term “bodily fluid” as used herein, generally refers to a natural fluid found in one of the fluid compartments of the human body. The principal fluid compartments are intracellular and extracellular. A much smaller segment, the transcellular, includes fluid in the tracheobronchial tree, the gastrointestinal tract, and the bladder; cerebrospinal fluid; and the aqueous humor of the eye. The bodily fluid includes blood plasma, serum, cerebrospinal fluid, or saliva. In an embodiment, the bodily fluid contains neutrophils and macrophages.

The term “body chemicals” as used herein, generally refers to chemicals existing in any one of the bodily fluids, neutrophil media, macrophage media or any complete cell growth media.

The term “biocompatibility” as used herein, refers to the capability of a material implanted in the body to exist in harmony with tissue without causing deleterious changes.

The term “biocompatible polymer” as used herein, generally refers to materials that are intended to interface with biological systems to evaluate, treat, augment or replace any tissue, organ or function of the body. Some of the characteristic properties of the biocompatible materials include “not having toxic or injurious effects on biological systems”, “the ability of a material to perform with an appropriate host response in a specific application”, and “ability of a biomaterial to perform its desired function with respect to a medical therapy, without eliciting any undesirable local or systemic effects in the recipient or beneficiary of that therapy, but generating the most appropriate beneficial cellular or tissue response in that specific situation, and optimizing the clinically relevant performance of that therapy”.

The term “biodegradable” as used herein, refers to polymers that degrade fully (i.e., down to monomeric species) under physiological or endosomal conditions. Biodegradable polymers are not necessarily hydrolytically degradable and may require enzymatic action to fully degrade.

The term “chromophore” as used herein refers to a chemical group (such as a xanthene group, or an acridine group) that absorbs light at a specific frequency and so imparts color to a molecule.

The term “colorant” as used herein is used interchangeably with the term “chromophore compound” throughout the entire disclosure.

The term “dye” as used herein includes both the colorant and the IR absorbing agent.

The term “IR absorbing material”, “IR dye”, “infrared radiation absorbing agent”, and “IR absorbing agent” as used herein are used interchangeably.

The term “Efficacy Determination Protocol” as used herein, generally refers to the method used for determining the degree of the degradation of a colorant and/or a material inside a particle, wherein the material interacts with an exogenous source, after being treated with body chemicals for a period of time that simulates the use environment (e.g., tattoo). Various analytical tools, like UV-VIS-NIR, NMR, HPLC, LCMS, etc., would be used to quantify the concentration of the colorant in the extracts and control. The details of the Efficacy Determination Protocol are described in Example 6. In some instances, if the degradation of the colorant is less than 90% and the degradation of the material is less than 90%, then the particle is considered passing the Efficacy Determination Protocol. In some instances, depending on the absorption intensity of the colorant and the physicochemical property of the material, if the degradation of the colorant is less than 85%, 80%, 75%, 70%, 65%, 60%, 55%, 50%, 45%, 40%, 35%, 30%, 25%, 20%, 15%, 10%, or 5%, and the degradation of the material is less than 85%, 80%, 75%, 70%, 65%, 60%, 55%, 50%, 45%, 40%, 35%, 30%, 25%, 20%, 15%, 10%, or 5%, then the particle is considered passing the Efficacy Determination Protocol.

The term “Extractable Cytotoxicity Test” as used herein, generally refers to an in vitro leaching protocol (using physiologically relevant media that contains serum proteins at physiological temperature) can be used to extract the colorants from the particles. The extract can then be used as is (“neat” or 1×) or in serial dilutions (up to 0.0001× dilutions) with the media in a cytotoxicity test against healthy cells (different cells will be chosen depending upon the application) as a surrogate measurement for the porosity of the particles. The neat or dilution of the extract that kills 30% of the cells can be measured and is referred to as an IC₃₀. Likewise, the neat or dilution of the extract that kills 10% of the cells can be measured and is referred to as an IC₁₀. The neat or dilution of the extract that kills 20% of the cells or below can be measured and is referred to as an IC₂₀. The neat or dilution of the extract that kills 40% or below of the cells can be measured and is referred to as an IC₄₀. The neat or dilution of the extract that kills 50% or below of the cells can be measured and is referred to as an IC₅₀. The neat or dilution of the extract that kills 60% or below of the cells can be measured and is referred to as an IC₆₀. The neat or dilution of the extract that kills 70% or below of the cells can be measured and is referred to as an IC₇₀. The neat or dilution of the extract that kills 80% or below of the cells can be measured and is referred to as an IC₈₀. The neat or dilution of the extract that kills 90% or below of the cells can be measured and is referred to as an IC₉₀. Details of the Extractable Cytotoxicity Test are described in Example 4. The Extractable Cytotoxicity Test is compliant with the international standards: ISO-10993-5 “Tests for cytotoxicity- in vitro methods”. In some instances, if the neat or dilution concentrations of the colorant and of the material in the leachate are independently less than IC₁₀, IC₃₀, IC₄₀, IC₅₀, IC₆₀, IC₇₀, IC₈₀, or IC₉₀, the particle passes the Extractable Cytotoxicity Test.

The term “feedback loop” as used herein, generally refers to a feedback loop based on the Extractable Cytotoxicity Test and/or the Efficacy Determination Protocol which have been utilized to evaluate if a particle needs to be rendered less porous by altering the chemistry of the particle fabrication. In an embodiment, in the Extractable Cytotoxicity Test, when cell death is less than or equal to 30% then the particles are considered to have passed the Extractable Cytotoxicity Test. The Extractable Cytotoxicity Test is compliant with the international standards: ISO-10993-5 “Tests for cytotoxicity- in vitro methods”. In some embodiments, when cell death is less than or equal to 10%, 20%, 40%, 50%, 60%, 70%, 80%, or 90%, then the particles are considered to have passed the corresponding Extractable Cytotoxicity Test.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

The term “macrophage medium” as used herein, generally refers to a complete medium designed for the culture of macrophages. The medium consists of basal medium (containing essential and non-essential amino acids, vitamins, organic and inorganic compounds, hormones, growth factors, trace minerals), supplemented with macrophage growth supplement, antibiotics, and fetal bovine serum.

The term “the material” as used herein, refers to the material that interacts with an exogenous source described in the disclosure.

The term “Material Process Stability” as used herein refers to the preservation of the optical and physical characteristics of the material under conditions of use such that it can deliver heat as intended upon stimulation by the exogenous source.

The term “neutrophil medium” as used herein, generally refers to a complete medium designed for the culture of neutrophils. The medium contains a basal medium (containing essential and non-essential amino acids, vitamins, organic and inorganic compounds, hormones, growth factors, trace minerals), supplemented with neutrophil culture supplement, antibiotics (i.e. penicillin, streptomycin), L-glutamine, and fetal bovine serum (FBS).

The term polymer “polydispersity (PD)” as used herein, generally is used as a measure of the broadness of a molecular weight distribution of a polymer and is defined by the formula

$$PD = \frac{Mw}{Mn} .$$

The larger the polydispersity, the broader the molecular weight. A monodisperse polymer where all the chain lengths are equal (such as endogenous protein) has an Mw/Mn=1. The best controlled synthetic polymers have Mw/Mn of 1.02 to 1.10.

The term “Polydispersity Index (PdI)” is defined as the square of the ratio of standard deviation (σ) of the particle diameter distribution divided by the mean particle diameter (2a), as illustrated by the formula: PdI=(σ/2a)². PdI is used to estimate the degree of non-uniformity of a size distribution of particles, and larger PdI values correspond to a larger size distribution in the particle sample. PdI can also indicate nanoparticle aggregation along with the consistency and efficiency of particle surface modifications. A sample is considered monodisperse when the PdI value is less than 0.1.

The term “solid solution” as used herein, refers to the colorant molecularly dissolved in the solid excipient matrix such as hydrophobic polymers, wherein the colorant is miscible with the polymer matrix excipient.

The term “solid dispersion” as used herein, refers to the colorant dispersed as crystalline or amorphous particles, wherein the colorant is dispersed in an amorphous polymer and is distributed at random between the polymer matrix excipient.

Stöber reaction: The Stöber reaction was reported by Werner Stöber in 1968, and it remains today the most widely used wet chemistry synthetic approach to prepare silica (SiO₂) particles of controllable and uniform size. It is an example of a sol-gel process wherein a molecular precursor (typically tetraethylorthosilicate, TEOS) is first reacted with water in an alcoholic solution, the resulting molecules then joining together to build larger cross-linked inorganic network structures. The particles in this disclosure used a modified Stöber process using vinyltrimethoxysilane (VTMS) reagent. In 1999 a two-stage modification was reported that allowed the controlled formation of silica particles with small pores. The process was undertaken at low pH in the presence of a surface-active molecule. The hydrolysis step is completed with the formation of a microemulsion before adding sodium fluoride to start the condensation process. Development work has also been undertaken for larger pore structures such as macroporous monoliths, core-shell particles based on polystyrene, cyclen, or polyamines, and carbon spheres.

### 1. Tattoo Particles

This disclosure provides tattoo particles useful for the removable tissue marking by rendering the colored tattoo pigments colorless when activated by the exogenous source.

In one embodiment, this disclosure provides for tissue markings that are suitable for permanent markings and are capable of being selectively being rendered colorless, as desired, on demand. In some embodiments, the tissue markings may be produced from ink formulations prepared from the tattoo pigment particles, and the particles can be rendered colorless by irradiation with a suitable infrared light source.

In one embodiment, this disclosure provides a particle suitable for tissue marking applications (e.g., tattoo ink) comprising: (a) a core comprising a carrier, a material, and a colorant, (b) a shell enclosing the core, wherein the material absorbs radiation at infrared wavelengths (IR absorbing agent), wherein the colorant dye and the material exhibits stability such that the colorant and the material in the particle exhibit stability such that the particle is considered passing the Efficacy Determination Protocol; and wherein the particle structure is constructed such that it passes the Extractable Cytotoxicity Test.

To be useful for tissue marking, the colorant encapsulated within the particles must be rendered sufficiently non-toxic. Cytotoxicity can be tested by the survival of cell lines when they are exposed to relevant chemical agents. In particular, ink particles can be extracted with physiologically relevant media that contains serum proteins at physiological temperature, and these extracts tested for cytotoxicity against healthy cells. The concentration of the extract that kills 30% of the cells can be estimated and is referred to as an IC₃₀. An IC₃₀ of the particles can be established for every application. Such an “Extractable Cytotoxicity Test” is a direct measure of the toxicological impact of tattoo ink components, and methods must be taken beyond those described in the literature to assure the safe composition of these inks. These methods can include additional chemistry to incorporate additional coatings that reduce the incursion of biological media. Most importantly, the process of preparing ink particles is not complete until testing reveals that the composition meets a required toxicological specification.

In one embodiment, this disclosure provides for tissue markings that are suitable for permanent markings and are capable of being selectively being rendered colorless, as desired, on demand. In some embodiments, the tissue markings may be produced from ink formulations prepared from the tattoo pigment particles, and the particles can be rendered colorless by irradiation with a suitable infrared light source.

In an embodiment, the degradation of the colorant and the material respectively inside the particle due to incursion by body chemicals is less than 85%, 80%, 75%, 70%, 65%, 60%, 55%, 50%, 45%, 40%, 35%, 30%, 25%, 20%, 15%, 10%, or 5% measured by the Efficacy Determination Protocol.

The polymeric particles may have a certain free volume or porosity associated with them depending upon the nature of the polymeric carrier used and encapsulation methodology and efficacy. Therefore, the colorant and/or the material responsive to the exogenous source may be susceptible to degradation due to the incursion of the body chemicals into the interior of the particles. Such degradation reduces the stability of the encapsulated colorant and/or the material. On the other hand, the colorant and/or the material may leach out or diffuse to the outside of the particle due to the carrier porosity or free volume. This leakage can lead to the cytotoxicity if the particles are implanted in a human subject.

It should be noted that the carrier matrix alone may be insufficient to provide the barrier protection from the penetration of the body chemicals outside the particle, nor to prevent the leakage of the colorant and/or the material due to the inherent porosity of the carrier matrix.

Thus, in some embodiments, the disclosure provides a particle with a shell having suitable barrier properties for limiting the exposure of the colorant and/or the material inside the particles to the body chemicals, and also reducing the colorant and/or the material leaching out or diffusing to the outside of the particle.

Therefore, in some embodiments, the present disclosure provides particles having a core-shell structure to reduce particle porosity and to protect the enclosed colorant and/or the material from the degradation by the body chemicals. Therefore, the stability of the colorant and/or the material inside the particles are improved due to the reduced incursion of the body chemicals. Also, the cytotoxicity of the particles due to the leakage of the colorant and/or the material is minimized by the process described in FIG. 1.

In some embodiments, the particles are biocompatible and/or biodegradable.

In some embodiments, the carrier of the particles is selected to be compatible with the colorant and/or the material so as to maximize efficacy. For example, in the case wherein the material is an infrared dye, a solid solution of the material with the carrier will maximize its absorption density. In the absence of a solid solution, especially when the material is an organic dye, can lead to aggregation, loss of absorption density and shift in absorption maximum which can limit interaction with the exogenous source in undesirable ways

In an embodiment, after exposure to the exogenous source the particles retain their structural integrity, and the colorant and the material that interacts with exogenous source are retained inside the particle. Colorants could include dyes and pigments.

In some embodiments, the particles are amorphous or partially amorphous or partially crystalline.

### (a) Colorant

In one embodiment, this disclosure provides a particle suitable for tissue marking applications (e.g., tattoo ink) comprising (a) a core comprising a carrier, a material, and a thermally-activatable colorant, (b) a shell enclosing the core, wherein the material absorbs radiation at infrared wavelengths (IR absorbing agent), wherein the thermally-activatable colorant and the material exhibits stability such that the particle is considered passing the Efficacy Determination Protocol; and wherein the particle structure is constructed such that it passes the Extractable Cytotoxicity Test.

In some embodiments, the thermally activatable colorant comprises a chromophore group and a thermally activatable fragmentation group. In some embodiments, the thermally-activatable colorant is chosen such that it contains a chromophore group conjugated with a nucleophilic group that is protected by a thermally activatable fragmentation group. Upon application of sufficient heat generated by the IR absorbing agent within the particle, the thermally activatable fragmentation group is degraded to produce the nucleophilic group that subsequently reacts with the chromophore group to eliminate the visible light absorption of the colorant responsible for its color. In some embodiments, the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof. In some embodiments, the chromophore group is selected from a substituted or unsubstituted xanthene such as those described in U.S. Pat. Nos. 4,602,263, and 4,826,976, and in EP 174054, the disclosures of which are incorporated herein by reference by its entirety. In some embodiments, the substituted or unsubstituted xanthene chromophore is one or more selected from 6-(dibutylamino)-9-[2-[[[(1,1-dimethylethoxy)carbonyl](methylsulfonyl)amino]methyl]phenyl]-3-methyl-2-(phenylamino)-xanthylium (B141), 3,6-bis(2,3-dihydro-1H-indol-1-yl)-9-[2-[[[(1,1-dimethylethoxy)carbonyl]methylamino]sulfonyl]phenyl]-xanthylium (C161: CAS Number 104434-22-2); 3,6-bis[(2-chlorophenyl)methylamino]-9-[2-[[[(1,1-dimethylethoxy)carbonyl]methylamino]sulfonyl]phenyl]-xanthylium (M071: CAS Number 104434-20-0), 9-[2-[[[(1,1-dimethylethoxy)carbonyl]methylamino]sulfonyl]phenyl]-3,6-diphenoxy-xanthylium (Y161: CAS Number 104434-14-2). In some embodiments, the particle comprises a mixture of chromophore compounds containing B141 (black), M071 (magenta), C161 (cyan), or Y161 (yellow).

In some embodiments, the colorant having the substituted or unsubstituted xanthene chromophore conjugated with a thermally activatable fragmentation is selected from those described in U.S. Pat. No. 8,039,193, the disclosures of which are incorporated herein by reference by its entirety.

In some embodiments, the colorant is a colored compound having formula D-Sp-Nu-FG as described in U.S. Pat. No. 8,039,193 comprising a thermally activatable fragmentation group, wherein D comprises a chromophore, Sp comprises an optional spacer group connecting the colored compound and a nucleophile Nu, Nu comprises a substituted or unsubstituted nucleophilic group and FG comprises the thermally activatable fragmentation group; and wherein the at least one colored compound is capable of being rendered colorless by unimolecular fragmentation of the thermally activatable fragmentation group when the colored compound is exposed to a sufficient dose of an exogenous source to render the colored compound colorless when desired. In some embodiments, the exogenous source is infrared radiation. In some embodiments, the at least one colored compound further comprises one or more ballast groups attached to a chromophore, the thermally activatable fragmentation group or both.

In some embodiments, the chromophore D comprises a substituted or unsubstituted triarylmethane, a xanthene, a rhodamine, a fluoran, an azocarbocyanine, a benzylidene, a thiazine, an acridine, an aminoanthraquinone or other chromophore; the optional spacer group Sp comprises substituted or unsubstituted alkyl groups, cycloaliphatic groups, sulfur or phosphorus containing groups or their oxides, or other spacing groups; the nucleophile group Nu comprises substituted or unsubstituted, oxygen, sulfur, phosphorus, carbon, selenium, nitrogen or silicon, or oxides thereof, or other substituted or unsubstituted nucleophilic group; and the thermally activatable fragmentation group FG comprises substituted and unsubstituted carbonates, carbamates, esters, lactams, lactones, amides, imides, oximes, sulfonates, sulfates, sulfenates, phosphates, phosphonates, or —R₂C—RHC—Y, wherein R is the same or different and comprises substituted or unsubstituted alkyl, aryl, or aralkyl groups and wherein Y comprises cyano, nitro, a sulfoxide, a carbonyl containing compound, a sulfonamide or other electron-withdrawing substituent that is capable of fragmenting when a sufficient amount of thermal energy is applied.

In some embodiments, the colored compound having formula D-Sp-Nu-FG is a compound selected from the compounds having formulas (I)-(VII):

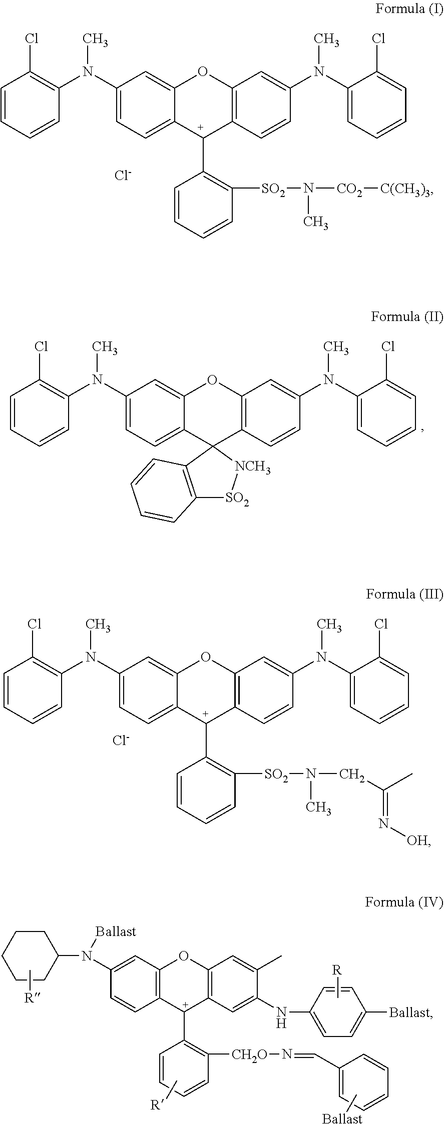

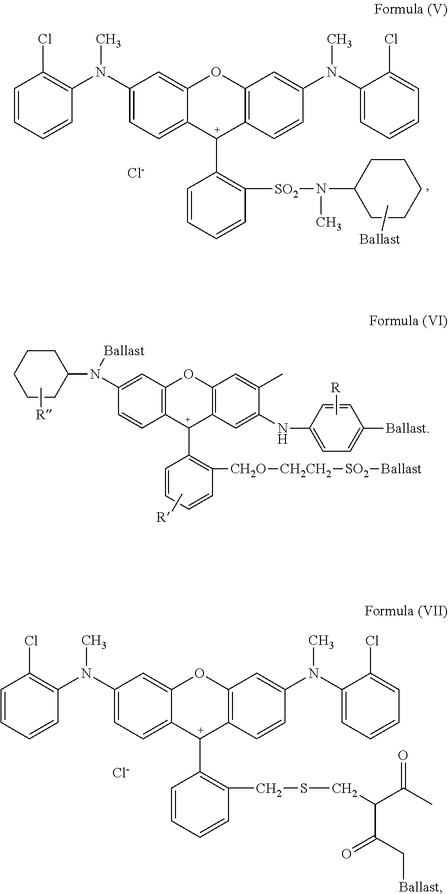

and salts thereof, and combinations thereof.

In some embodiments, the carrier forms a matrix. In some embodiments, the colorant admixed with the carrier forms a homogeneous dispersion or a solid solution.

In some embodiments, the particle has a loading amount of the colorant that is measured by spectroscopic absorbance. In some embodiments, the particle has a loading amount of the colorant that is measured by known analytical technology in the art, like UV-VIS-NIR, NMR, HPLC, LCMS, etc. In some embodiments, the colorant loading amount is in a range from about 0.01 wt. % to about 95.0 wt. % by the total weight of the particle. In some embodiments, the colorant loading amount is in a range from about 0.01 wt. % to about 20.0 wt. % by the total weight of the particle. In some embodiments, the particle has the colorant loading amount in a range from about 1.0 wt. % to about 20.0 wt. %. In some embodiments, the particle has the colorant loading amount in a range from about 5.0 wt. % to about 20.0 wt. %. In some embodiments, the particle has the colorant loading amount in a range from about 10.0 wt. % to about 20.0 wt. %. In some embodiments, the particle having the colorant loading amount in a range from about 5.0 wt. % to about 15.0 wt. %. In some embodiments, the particle has the colorant loading amount in a range from about 10.0 wt. % to about 15.0 wt. %. In some embodiments, the particle has the colorant loading amount in a range from about 5.0 wt. % to about 12.5 wt. %. In some embodiments, the colorant loading amount is a value selected from the group of: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7. 5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %. In some embodiments, the particle has the colorant loading amount of about 12.5 wt. %. In some embodiments, the colorant loading amount is a value selected from the group of: about 0.1 wt. %, about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 15.0 wt. %, about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, or about 95.0 wt. %.

In some embodiments, the particle contains B141 and M071 chromophore compounds at a weight ratio of 0.14 M071 to 1.0 of B141 to balance out the green hue of the black dye (“neutral black”). In some embodiments, the particle contains B141, M071 and additionally Y161 to balance out the blue. In some embodiments, the particle contains B141, M071 and additionally C161 along with an increased weight amount of M071 to increase the black color intensity (“process black”). In some embodiments, the particles contain a mixture of dyes comprising from about 6.2 wt. % to about 10.0 wt. % B141, from about 1.36 wt. % to about 2.71 wt. % M071, from about 0 wt. % to about 2.71 wt. % C161, from about 0 wt. % to about 4.52 wt. % Y161 and from about 10.4 wt. % to about 14.0 wt. % Epolight™ 1117. In some embodiments, the particle comprises B141, M071, C161 and Y161 at a weight ratio of 1.0 B141, 0.3 M071, 0.3 C161, and 0.5 Y161. The compositions for black dyes having a range of color strength and vibrancy are summarized in Table 1 below.

TABLE 1
Compositions of Particles with Various Dye Combinations

| | | B805 | Cyanox ® | B141 | M071 | C161 | Y161 | | IR1117 | Polymer/ |
|---|---|---|---|---|---|---|---|---|---|---|
| | | (wt. | 1790 (wt. | (wt. | (wt. | (wt. | (wt. | Y184/ | (wt. | dye |
| Entry | Composition | %) | %) | %) | %) | %) | %) | Y197 | %) | weight ratio |
| 1 | Neutral | 75 | 0 | 10 | 1.36 | 0 | 0 | 0 | 13.64 | 3:1 |
| | Black (NB) | | | | | | | | | |
| 2 | Process | 75 | 0 | 8.08 | 2.42 | 2.42 | 0 | 0 | 12.1 | 3:1 |
| | Black 1 | | | | | | | | | |
| | (PB1) | | | | | | | | | |
| 3 | PB2 | 75 | 0 | 6.94 | 2.08 | 2.08 | 3.47 | 0 | 10.4 | 3:1 |
| 4 | PB3 | 70 | 5 | 6.2 | 1.86 | 1.86 | 3.1 | 0 | 12 | 3:1^{a} |
| 5 | PB4 | 62 | 5 | 9.05 | 2.71 | 2.71 | 4.52 | 0 | 14 | 2:1^{a} |
| 6 | Cyan | 78.3 | 5 | 0 | 0 | 11.11 | 0 | 0 | 5.56 | 5:1^{a} |
| 7 | Magenta | 78.3 | 5 | 0 | 11.11 | 0 | 0 | 0 | 5.56 | 5:1^{a} |
| 8 | Yellow | 78.3 | 5 | 0 | 0 | 0 | 11.11 | 0 | 5.56 | 5:1^{a} |
| 9 | PBS(black) | 61.25 | 5 | 9.92 | 1.5 | 2.97 | 0 | 4.96 Y184 | 14 | 2:1 |
| 10 | Cyan | 76.25 | 5 | 0 | 0 | 12.5 | 0 | 0 | 6.25 | 4:1 |
| 11 | Magenta | 74.50 | 5 | 0 | 12.5 | 0 | 0 | 0 | 8 | 4:1 |
| 12 | Orange | 74.50 | 5 | 0 | 0 | 0 | 0 | 12.5 Y184 | 8 | 4:1 |
| 13 | Yellow | 76.25 | 5 | 0 | 0 | 0 | 0 | 12.5 Y197 | 6.25 | 4:1 |

^{a}The amount of polymer used to calculate the polymer/dye weight ratio includes the weight amount of the antioxidant Cyanox ® 1790.

In some embodiments, the particle is a PB5 (black) particle comprising: about 61.65 wt. % B-805 polymer, about 2.97 wt. % C161, about 1.5 wt. % M071, about 9.92 wt. % B141, about 4.96 wt. % Y184, about 14.0 wt. % Epolight™ IR 1117, and about 5.0 wt. % Cyanox® by the total weight of the particle.

In some embodiments, the particle is a Cyan particle comprising: about 76.25 wt. % B-805 polymer, about 12.5 wt. % C161, about 6.25 wt. % Epolight™ IR 1117, and about 5.0 wt. % Cyanox® by the total weight of the particle.

In some embodiments, the particle is a yellow particle comprising: about 76.25 wt. % B-805 polymer, about 12.5 wt. % Y197, about 6.25 wt. % Epolight™ IR 1117, and about 5.0 wt. % Cyanox® by the total weight of the particle.

In some embodiments, the particle is a Magenta particle comprising: about 74.50 wt. % B-805 polymer, about 12.5 wt. % M071, about 8.0 wt. % Epolight™ IR 1117, and about 5.0 wt. % Cyanox® by the total weight of the particle.

In some embodiments, the particle is an Orange particle comprising: about 74.50 wt. % B-805 polymer, about 12.5 wt. % Y184, about 8.0 wt. % Epolight™ IR 1117, and about 5.0 wt. % Cyanox® by the total weight of the particle.

In some embodiments, the particle is a Magenta particle comprising: about 74.50 wt. % B-805 polymer, about 6.25 wt. % M071, about 8.0 wt. % Epolight™ IR 1117, and about 5.0 wt. % Cyanox® by the total weight of the particle.

In some embodiments, the particle is a Magenta particle comprising: about 74.50 wt. % B-805 polymer, about 3.0 wt. % M071, about 8.0 wt. % Epolight™ IR 1117, and about 5.0 wt. % Cyanox® by the total weight of the particle.

In some embodiments, the particle is a Magenta particle comprising: about 74.50 wt. % B-805 polymer, about 1.0 wt. % M071, about 8.0 wt. % Epolight™ IR 1117, and about 5.0 wt. % Cyanox® by the total weight of the particle.

In some embodiments, the particle is a Cyan particle comprising: about 76.25 wt. % B-805 polymer, about 6.25 wt. % C161, about 6.25 wt. % Epolight™ IR 1117, and about 5.0 wt. % Cyanox® by the total weight of the particle.

In some embodiments, the particle is a Cyan particle comprising: about 76.25 wt. % B-805 polymer, about 4.0 wt. % C161, about 6.25 wt. % Epolight™ IR 1117, and about 5.0 wt. % Cyanox® by the total weight of the particle.

In some embodiments, the particle is a Cyan particle comprising: about 76.25 wt. % B-805 polymer, about 2.0 wt. % C161, about 6.25 wt. % Epolight™ IR 1117, and about 5.0 wt. % Cyanox® by the total weight of the particle.

In some embodiments, the particle is an Orange particle comprising: about 74.50 wt. % B-805 polymer, about 6.25 wt. % Y184, about 8.0 wt. % Epolight™ IR 1117, and about 5.0 wt. % Cyanox® by the total weight of the particle.

In some embodiments, the particle is an Orange particle comprising: about 74.50 wt. % B-805 polymer, about 3.0 wt. % Y184, about 8.0 wt. % Epolight™ IR 1117, and about 5.0 wt. % Cyanox® by the total weight of the particle.

In some embodiments, the chromophore compound (dye) is present in an amount ranging selected from the group of: 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 9.0 wt. %, 9.5 wt. %, 10.0 wt. %, 10.5 wt. %, 11.0 wt. %, 11.5 wt. %, 12.0 wt. %, 12.125 wt. %, 12.25 wt. %, 12.50 wt. %, 12.625 wt. %, 12.75 wt. %, 13.0 wt. %, 13.125 wt. %, 13.25 wt. %, 13.5 wt. %, 13.625 wt. %, 13.75 wt. %, 14.0 wt. %,14.125 wt. %, 14.25 wt. %, 14.50 wt. %, 14.625 wt. %, 14.75 wt. %, 15.0 wt. %, 15.125 wt. %, 15.25 wt. %, 15.5 wt. %, 15.625 wt. %, 15.75 wt. %, 16.0 wt. %,16.125 wt. %, 16.25 wt. %, 16.50 wt. %, 16.625 wt. %, 16.75 wt. %, 17.0 wt. %, 17.125 wt. %, 17.25 wt. %, 17.5 wt. %, 17.625 wt. %, 17.75 wt. %, 18.0 wt. %, 18.125 wt. %, 18.25 wt. %, 18.5 wt. %, 18.625 wt. %, 18.75 wt. %, 19.0 wt. %, 19.125 wt. %, 19.25 wt. %, 19.5 wt. %, 19.625 wt. %, 19.75 wt. %, 20.0 wt. %, 20.125 wt. %, 20.25 wt. %, 20.5 wt. %, 20.625 wt. %, 20.75 wt. %, 21.0 wt. %, 21.125 wt. %, 21.25 wt. %, 21.5 wt. %, 21.625 wt. %, 21.75 wt. %, 22.0 wt. %, 22.125 wt. %, 22.25 wt. %, 22.5 wt. %, 22.625 wt. %, 22.75 wt. %, 23.0 wt. %, 23.125 wt. %, 23.25 wt. %, 23.5 wt. %, 23.625 wt. %, 23.75 wt. %, 24.0 wt. %, 24.125 wt. %, 24.25 wt. %, 24.5 wt. %, 24.625 wt. %, 24.75 wt. %, 25.0 wt. %, 25.125 wt. %, 25.25 wt. %, 25.5 wt. %, 25.625 wt. %, 25.75 wt. %, 26.0 wt. %, 26.125 wt. %, 26.25 wt. %, 26.5 wt. %, 26.625 wt. %, 26.75 wt. %, 27.0 wt. %, 27.125 wt. %, 27.25 wt. %, 27.5 wt. %, 27.625 wt. %, 27.75 wt. %, 28.0 wt. %, 28.125 wt. %, 28.25 wt. %, 28.5 wt. %, 28.625 wt. %, 28.75 wt. %, 29.0 wt. %, 29.125 wt. %, 29.25 wt. %, 29.5 wt. %, 29.625 wt. %, 29.75 wt. %, 30.0 wt. %, 30.125 wt. %, 30.25 wt. %, 30.5 wt. %, 30.625 wt. %, 30.75 wt. %, 31.0 wt. %, 31.125 wt. %, 31.25 wt. %, 31.5 wt. %, 31.625 wt. %, 31.75 wt. %, 32.0 wt. %, 32.125 wt. %, 32.25 wt. %, 32.5 wt. %, 32.625 wt. %, 32.75 wt. %, 33.0 wt. %, 33.125 wt. %, 33.25 wt. %, 33.5 wt. %, 33.625 wt. %, 33.75 wt. %, 34.0 wt. %, 34.125 wt. %, 34.25 wt. %, 34.5 wt. %, 34.625 wt. %, 34.75 wt. %, or 35.0 wt. % by the total weight of the particle. In some embodiments, the loading amount of the chromophore compound can be as high as 33.0 wt. % by the total weight of the particle without compromising the particle structure integrity but maintaining strong color strength. In some embodiments, a single chromophore compound is present in an amount ranging from 1.0 wt. % to 15.0 wt. %. In some embodiments, the single chromophore compound in the particle is present in an amount selected from the group of: 1.0 wt. %, 1.36 wt. %, 1. 86 wt. %, 2.08 wt. %, 2.42 wt. %, 2.71 wt. %, 3.1 wt. %, 3.47 wt. %, 4.52 wt. %, 5.56 wt. %, 6.20 wt. %, 6.94 wt. %, 8.08 w. %, 9.05 wt. %, 10.0 wt. %, 11.11 wt. %, 12.0 wt. %, 12.1 wt. %, 13.64 wt. %, 14.0 wt. %, or 15.0 wt. % by the total weight of the particle.

In some embodiments, the chromophore compound is present in an amount ranging from about 10.0 wt. % to about 33.0 wt. % by total weight of the particle. In some embodiments, the chromophore compound is present in an amount ranging from about 12.5 wt. % to about 33.0 wt. % by total weight of the particle to provide a black color that is comparable with the color vibrancy and intensity of the commercially available black pigment. In some embodiments, the chromophore compound is present in an amount ranging from about 10.0 wt. % to about 30.0 wt. % by total weight of the particle. In some embodiments, the chromophore compound is present in an amount ranging from about 10.0 wt. % to about 25.0 wt. % by total weight of the particle. In some embodiments, the chromophore compound is present in an amount ranging from about 12.5 wt. % to about 25.0 wt. % by total weight of the particle. In some embodiments, the chromophore compound is present in an amount ranging from 12.5 wt. % to 20.0 wt. % by total weight of the particle and exhibits superior color strength. In some embodiments, the chromophore compound is present in an amount ranging from about 12.5 wt. % to about 17.0 wt. % by total weight of the particle. In some embodiments, the chromophore compound is present in an amount ranging from about 12.5 wt. % to about 15.0 wt. % by total weight of the particle. In some embodiments, the chromophore compound is present in an amount ranging from about 10.0 wt. % to about 20.0 wt. % by total weight of the particle. In some embodiments, the chromophore compound is present in an amount ranging from about 10.0 wt. % to about 15.0 wt. % by total weight of the particle.

In some embodiments, the chromophore compound is present in an amount selected from the group of about 10.0 wt. %, about 11.0 wt. %, about 12.0 wt. %, about 13.0 wt. %, about 14.0 wt. %, about 15.0 wt. %, about 16.0 wt. %, about 17.0 wt. %, about 18.0 wt. %, about 19.0 wt. %, about 20.0 wt. %, about 21.0 wt. %, about 22.0 wt. %, about 23.0 wt. %, about 24.0 wt. %, about 25.0 wt. %, about 26.0 wt. %, about 27.0 wt. %, about 28.0 wt. %, about 29.0 wt. %, about 30.0 wt. %, about 31.0 wt. %, about 32.0 wt. %, and about 33.0 wt. % by total weight of the particle.

The concentration of dye is related to the perception of the color of the particle. The visible dye contents can be used as high as 20.0 wt. % by the total weight of the particle without compromising the particle color strength due to broadening of the dye's absorbance and lower peak absorption resulting from the increased load amount of chromophore compounds. In some embodiments, the particle exhibits superior color strength with chromophore compounds having a loading at about 12.5 wt. % by the total weight of the particle. In some embodiments, the particle exhibits superior color strength with chromophore compounds having a loading at about 13.0 wt. % by the total weight of the particle. In some embodiments, the particle exhibits superior color strength with chromophore compounds having a loading at about 13.5 wt. % by the total weight of the particle. In some embodiments, the particle exhibits superior color strength with chromophore compounds having a loading at about 14.0 wt. % by the total weight of the particle. In some embodiments, the particle exhibits superior color strength with chromophore compounds having a loading at about 14.5 wt. % by the total weight of the particle. In some embodiments, the particle exhibits superior color strength with chromophore compounds having a loading at about 15.0 wt. % by the total weight of the particle. In some embodiments, the particle exhibits superior color strength with chromophore compounds having a loading at about 15.5 wt. % by the total weight of the particle. In some embodiments, the particle exhibits superior color strength with chromophore compounds having a loading at about 16.0 wt. % by the total weight of the particle. In some embodiments, the particle exhibits superior color strength with chromophore compounds having a loading at about 16.5 wt. % by the total weight of the particle. In some embodiments, the particle exhibits superior color strength with chromophore compounds having a loading at about 17.0 wt. % by the total weight of the particle. In some embodiments, the particle exhibits superior color strength with chromophore compounds having a loading at about 17.5 wt. % by the total weight of the particle. In some embodiments, the thermally activatable fragmentation group comprises substituted and unsubstituted carbonates, carbamates, esters, lactams, lactones, amides, imides, oximes, sulfonates, or phosphonates.

### (b) Carrier

To achieve the stability and the cytotoxicity criteria as set forth above, it is necessary to create particles that have the appropriate structural integrity or porosity. For a given agent and material, proper choice of the carrier is an important parameter to achieve appropriate structural integrity. It is also important to select a carrier that is compatible with the colorant and the material to be encapsulated because otherwise the colorant and material efficacy can be adversely impacted.

In some embodiments, the particles comprise a carrier. In an embodiment, the carrier may include a lipid selected from the group of lipid, polymer-lipid conjugate, carbohydrate-lipid conjugate, peptide-lipid conjugate, protein-lipid conjugate, and mixtures thereof. In some embodiments, the lipid may include one or more of the following: dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl- sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group of DPPC, MPPC, PEG, DMPC, DMPG, DSPE, DOPC, DOPE, DPPG, DSPC, DSPE-PEG, MSPC, cholesterol, PS, PC, PE, PG, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the carrier comprises 2 parts of 1,2-distearoyl-sn-glycero-3-phosphoglycerol (DSPG), 1 part of cholesterol, and 0.2 part of 1,2-distearoyl-sn-glycero-3-phosphoethanolamine (DSPE-PEG2000). In some embodiments, the carrier comprises 2 parts sphingomyelin (egg), 1 part cholesterol and 0.2 parts of 1,2-distearoyl-sn-glycero-3-phosphoethanolamine (DSPE-PEG2000).

In an embodiment, the carrier is a polymer. In some embodiments, the polymer carrier is biodegradable and/or biocompatible polymers. In some embodiments, the polymer carrier is selected based on the specific colorant to be encapsulated (payload), e.g., polymeric carrier is chemically compatible with the colorant. It should be noted that the use of a biocompatible carrier does not ensure that the particle with payloads will be biocompatible.

In some embodiments, the polymers may include, but are not limited to: polymethyl methacrylate, polyester, poly caprolactone (PCL), poly(trimethylene carbonate) or other poly (alpha-esters), polyurethanes, poly(allylamine hydrochloride), poly(ester amides), poly (ortho esters), polyanyhydrides, poly (anhydride-co-imide), crosslinked polyanhydrides, pseudo poly(amino acids), poly (alkylcyanoacrylates), polyphosphoesters, polyphosphazenes, chitosan, collagen, natural or synthetic poly(amino acids), elastin, elastin-like polypeptides, albumin, fibrin, polysiloxanes, polycarbosiloxanes, polysilazanes, polyalkoxysiloxanes, polysaccharides, cross-linkable polymers, thermo-responsive polymers, thermo-thinning polymers, thermo-thickening polymers, or block co-polymers of the above polymers with polyethylene glycol, and combinations thereof.

In some embodiments, the carrier comprises a hydrophobic polymer or copolymer of polymethacrylates, polycarbonate, or combinations thereof. In some embodiments, the carrier comprises polymethylmethacrylate (PMMA, Neocryl® 728 sold by DSM, T_{g}=111° C., acid value is of 6.5).

In some embodiments, the carrier comprises copolymer of two different methacrylate monomers. In some embodiments, the carrier comprises copolymer of methyl methacrylate monomer and C2-C6 alkyl methacrylate monomer. In some embodiments, the carrier comprises copolymer of methyl methacrylate monomer and C2-C4 alkyl methacrylate monomer. In some embodiments, the carrier comprises copolymer of methyl methacrylate monomer and C3-C4 alkyl methacrylate monomer. In some embodiments, the polymethacrylate copolymer is made from methyl methacrylate monomer and C4 alkyl methacrylate monomer. In some embodiments, the polymethacrylate copolymer is made from methyl methacrylate (MMA) monomer in an amount ranging from about 80.0 wt. % to about 99.0 wt. % and butyl methacrylate (BMA) monomer in an amount ranging from about 1.0 wt. % to about 20.0 wt. % by the total weight of the polymethacrylate copolymer. In some embodiments, the polymethacrylate copolymer is made from MMA monomer in an amount ranging from about 85.0 wt. % to about 96.0 wt. % and BMA monomer in an amount ranging from about 4.0 wt. % to about 15.0 wt. % by the total weight of the polymethacrylate copolymer. In some embodiments, the polymethacrylate copolymer is made from MMA monomer in an amount ranging from about 90.0 wt. % to about 96.0 wt. % and BMA monomer in an amount ranging from about 4.0 wt. % to about 10.0 wt. % by the total weight of the polymethacrylate copolymer. In some embodiments, the polymethacrylate copolymer is made from MMA monomer in an amount ranging from about 95.0 wt. % to about 96.0 wt. % and BMA monomer in an amount ranging from about 4.0 wt. % to about 5.0 wt. % by the total weight of the polymethacrylate copolymer. In some embodiments, the polymethacrylate copolymer is made from about 99.0 wt. % MMA monomer and about 1.0 wt. % BMA monomer by the total weight of the polymethacrylate copolymer. In some embodiments, the polymethacrylate copolymer is made from about 98.0 wt. % MMA monomer and about 2.0 wt. % BMA monomer by the total weight of the polymethacrylate copolymer. In some embodiments, the polymethacrylate copolymer is made from about 97.0 wt. % MMA monomer and about 3.0 wt. % BMA monomer by the total weight of the polymethacrylate copolymer. In some embodiments, the polymethacrylate copolymer is made from about 96.0 wt. % MMA monomer and about 4.0 wt. % BMA monomer by the total weight of the polymethacrylate copolymer. In some embodiments, the polymethacrylate copolymer is made from about 95.0 wt. % MMA monomer and about 5.0 wt. % BMA monomer by the total weight of the polymethacrylate copolymer. In some embodiments, the polymethacrylate copolymer is made from about 94.0 wt. % MMA monomer and about 6.0 wt. % BMA monomer by the total weight of the polymethacrylate copolymer.

In some embodiments, the weight ratio of the MMA repeating units to the BMA repeating units in the MMA/BMA copolymer is 80:20 to 99:1. In some embodiments, the weight ratio of the MMA repeating units to the BMA repeating units in the MMA/BMA copolymer is 85:15 to 96:4. In some embodiments, the weight ratio of the MMA repeating units to the BMA repeating units in the MMA/BMA copolymer is 90:10 to 96:4. In some embodiments, the weight ratio of the MMA repeating units to the BMA repeating units in the MMA/BMA copolymer is 95:5 to 96:4. In some embodiments, the weight ratio of the MMA repeating units to the BMA repeating units in the MMA/BMA copolymer is 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, or 99:1. In some embodiments, the polymethacrylate copolymer is MMA/BMA copolymer and the weight ratio of MMA to BMA is 96:4 (e.g., Neocryl® 805 by DSM, acid value less than 1).

In some embodiments, the hydrophobic polymethacrylate has an acid value less than 10. In some embodiments, the hydrophobic polymethacrylate has an acid value less than 5. In some embodiments, the hydrophobic polymethacrylate has an acid value less than 2. In some embodiments, the hydrophobic polymethacrylate has an acid value less than 1.

Depending upon the specific colorant and material encapsulated in the particle, to achieve desired porosity for minimizing leakage as well as reduce penetration of body fluids into the particle, it becomes necessary to incorporate cross-linkable groups such that with additional cross-linking the desired porosity can be achieved guided by the Efficacy Determination Protocol and the Extractable Cytotoxicity Test.

In some embodiments, the carrier comprises cross-linkable reactive groups selected from vinyl group (—CH═CH₂), ethynyl group (—CC—), vinyl dimethyl sulfone group, hydroxyl group (—OH), thiol group (—SH), amine group (—NH₂), aldehyde group (—CHO), carboxylic acid group (—COOH), and combinations thereof. In some embodiments, the carrier comprises the cross-linkable polysaccharides. In some embodiments, the cross-linkable polysaccharides may include alginic acid, sodium alginate, or carrageenan.

In some embodiments, for decreasing particle porosity, the carrier comprises cross-linked polymer networks resulted from reacting the cross-linkable reactive groups attached to the carrier with a cross-linker reagent. In some embodiments, the porosity or the free volume of the particle may be modified by reacting the carrier having cross-linkable reactive groups with a cross-linker reagent to form cross-linked carrier matrix, or by increasing the degree of cross-linking. In some embodiments, the degree of cross-linking can be tuned by controlling the weight ratio of the cross-linker reagent to the carrier having cross-linkable reactive groups in the cross-linking reaction.

In some embodiments, the cross-linker reagent for cross-linking hydroxyl group (—OH), thiol group (—SH), or amine group (—NH₂) attached to the carrier may include dithiobis(succinimidyl) propionate (Lomant's reagent), cystamine bisacrylamide, bisacryloyloxyethyl disulfide, N,N′-(ethane-1,2-diyl)diacrylamide, N,N′-(2-hydroxypropane-1,3-diyl)diacrylamide, polyisocyanate, polyisothiocyanate, dimethyl adipimidate, dimethyl pimelimidate, dimethyl suberimidate, dimethyl 3,3′-dithiobispropionimidate, glutaraldehyde, glyoxal, glyoxal-trimer dihydrate, dimethyl suberimidate, dimethyl 3,3′-dithiobispropionimidate glutaraldehyde, epoxides, bis-oxiranes, p-azidobenzoyl hydrazide, N-a-maleimidoacetoxy succinimide ester, p-azidophenyl glyoxal monohydrate, bis-((beta)-(4-azidosalicylamido)ethyl)disulfide, succinimidyl iodoacetate, succinimidyl 3-(bromoacetamido)propionate, 4-(iodoacetyl)aminobenzoate, N-α-maleimidoacet oxysuccinimide ester, N-β-maleimidopropyl oxysuccinimide ester, N-γ-maleimidobutyryl oxysuccinimide ester, m-maleimidobenzoyl-N-hydroxysuccinimide ester, succinimidyl 4-(N-maleimidomethyl)cyclohexane-1-carboxylate, N-ε-malemidocaproyl oxysuccinimide ester, succinimidyl 4-(p-maleimidophenyl)butyrate, succinimidyl 6-β-maleimidopropionamido)hexanoate, succinimidyl 3-(2-pyridyldithio)propionate (SPDP), PEG4-SPDP, PEG12-SPDP, disuccinimidyl tartrate, 4-succinimidyloxycarbonyl-α-methyl-α-(2-pyridyldithio)toluene, disuccinimidyl glutarate, ethylene glycol bis(succinimidylsuccinate), bis-(sulfosuccinimidyl) (ethylene glycol) bis(succinimidylsuccinate), bis-sulfosuccinimidyl suberate, disuccinimidyl-suberate, tris-succinimidyl aminotriacetate, diacylchlorides, or polyphenolic compounds (e.g., tannic acid or tannin as cross-linker for cross-linking protein such as collagen, gelatin etc., dopamine and its derivatives).

In some embodiments, the cross-linker reagent for cross-linking hydroxyl group (—OH), thiol group (—SH), or amine group (—NH₂) attached to the carrier may include carboxyl group terminated polyethylene glycol having 2-8 branching arms (used with carboxylic acid activation agent N-hydroxysuccinimide esters (NETS) and/or (1-ethyl-3-(3-dimethylaminopropyl)-carbodiimide (EDC)), for example, 4-arm PEG carboxyl (pentaerythritol core), 6-arm PEG carboxyl (hexaglycerin core), 8-arm PEG carboxyl (tripentaerythritol core). In some embodiments, the cross-linker reagent for cross-linking the hydroxyl group (—OH), thiol group (—SH), or amine group (—NH₂) attached to the carrier may include bis-succinimide ester terminated polyethylene glycol or star shaped succinimide ester terminated polyethylene glycol having 3-8 branching arms, for example, 4-arm PEG succinimidyl (pentaerythritol core) or 6-arm PEG succinimidyl (hexaglycerin core). In some embodiments, the succinimide ester, or carboxyl group terminated polyethylene glycol type cross-linker reagent may have a number average molecular weight ranging from about 150 Daltons (Da) to about 10 KDa. In some embodiments, the succinimide ester, or carboxyl group terminated polyethylene glycol type cross-linker reagent may have a number average molecular weight ranging from about 1 KDa to about 10 KDa. In some embodiments, the succinimide ester, or carboxyl group terminated polyethylene glycol type cross-linker reagent may have a number average molecular weight ranging from about 1 KDa to about 5 KDa. In some embodiments, the succinimide ester, or carboxyl group terminated polyethylene glycol type cross-linker reagent may have a number average molecular weight ranging from about 150 Da to about 1 KDa. In some embodiments, the succinimide ester, or carboxyl group terminated polyethylene glycol type cross-linker reagent may have a number average molecular weight ranging from about 150 Da to about 750 Da.

In some embodiments, the cross-linker reagent for cross-linking the reactive aldehyde group, vinyl dimethyl sulfone group, or carboxylic acid group (activation with N-hydroxysuccinimide esters (NHS) or (1-ethyl-3-(3-dimethylaminopropyl)-carbodiimide (EDC)) attached to the carrier may include polyamine compounds such as spermine, polyspermine, low molecular weight polyethylenimine (PEI), dilysine, liner or branched trilysine, tetralysine, pentalysine, hexylysine, heptalysine, octalysine, nonalysine, decalysine, undecalysine, dodecalysine, tridecalysine, tetradecalysine, pentadecalysine, or hyperbranched polylysines, polyols such as pentaerythritol, ethylene glycol, polyethylene glycol, glycerol, polyglycerol, sucrose, sorbitol etc.

In some embodiments, the cross-linker reagent for cross-linking the aldehyde group, vinyl dimethyl sulfone group, or carboxylic acid group (activation with N-hydroxysuccinimide esters (NHS) or (1-ethyl-3-(3-dimethylaminopropyl)-carbodiimide (EDC)) attached to the carrier may include amine terminated polyethylene glycols having 2-8 branching arms, for example, 4-arm PEG amine (pentaerythritol core), 6-arm PEG amine (hexaglycerin core), 8-arm PEG amine (tripentaerythritol core). In some embodiments, the amine terminated polyethylene glycol type cross-linker reagents may have a number average molecular weight ranging from 150 Da to 10 KDa. In some embodiments, the amine terminated polyethylene glycol type cross-linker reagents may have a number average molecular weight ranging from 1 KDa to 10 KDa. In some embodiments, the amine terminated polyethylene glycol type cross-linker reagents may have a number average molecular weight ranging from 1 KDa to 5 KDa. In some embodiments, the amine terminated polyethylene glycol type cross-linker reagents may have a number average molecular weight ranging from 150 Da to 1 KDa. In some embodiments, the amine terminated polyethylene glycol type cross-linker reagent may have a number average molecular weight ranging from 150 Da to 750 Da.

In some embodiments, the particle comprises the carrier to the colorant in a weight ratio ranging from 1:10 to 10:1. In some embodiments, the weight ratio of the carrier to the colorant ranges from 1:1 to 7:1. In some embodiments, the weight ratio of the carrier to the colorant is selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1. In some embodiments, the weight ratio of the carrier to the colorant is selected from the group of 1:1, 2:1, 3:1, 4:1, 5:1, 6:1, and 7:1. In some embodiments, the weight ratio of the carrier to the colorant is 2:1. In some embodiments, the weight ratio of the carrier to the colorant is 3:1. In some embodiments, the weight ratio of the carrier to the colorant is 4:1. In some embodiments, the weight ratio of the carrier to the colorant is 5:1. In some embodiments, the weight ratio of the carrier to the colorant is 6:1. In some embodiments, the weight ratio of the carrier to the colorant is 7:1.

(c) Material Interacting with an Exogenous Source

In an embodiment, the particles comprise a material interacting with an exogenous source (also known as exogenous trigger).

In some embodiments, the material interacting with the exogenous source can do something useful (e.g., produces heat or makes the particles more porous) that allows the colorant to perform its function.

In some embodiments, the exogenous source is electromagnetic radiation, microwaves, radio waves, sound waves, electrical, or magnetic field. In some embodiments, the exogenous source may be electromagnetic radiation (EMR).

In some embodiments, the material interacting with the exogenous source does not have significant optical absorption in the visible electromagnetic spectrum region (400 nm to 750 nm). In some embodiments, the colorant has absorption in the visible range (400 nm to 750 nm) and the material interacting with the exogenous source has significant absorption in the near infrared spectrum region (NIR) (750 nm to 1500 nm). In some embodiments, the material interacting with the exogenous source comprises a dye capable of absorbing electromagnetic radiation and converting the energy to heat (photothermal conversion). In some embodiments, the exogenous source comprises a laser light. In some embodiments, the laser light is a pulsed laser light. In some embodiments, the laser pulse duration is in a range from milliseconds to nanoseconds, and the laser has an oscillation wavelength at 1064 nm. In some embodiments, the laser emits light at 808 nm. In some embodiments, the laser emits light at 805 nm.

In some embodiments the exogenous source may have a cold tip to cool the target tissue area before, during and after application of the exogenous energy. In some embodiments the cold tip may be a temperature from 2-8° C.

In some embodiments, the material interacting with the exogenous source has significant absorption in the near infrared spectrum region (NIR). In some embodiments, the material interacting with the exogenous source has significant absorption in the NIR wavelength ranging from 700 nm to 1500 nm. In some embodiments, the material interacting with the exogenous source has significant absorption at a NIR wavelength in the range from 700 nm to 1400 nm. In some embodiments, the material interacting with the exogenous source has significant absorption at a NIR wavelength in the range from 700 nm to 1300 nm. In some embodiments, the material interacting with the exogenous source has significant absorption at a NIR wavelength in the range from 750 nm to 850 nm. In some embodiments, the material interacting with the exogenous source has significant absorption at a NIR wavelength in the range from 750 nm to 900 nm. In some embodiments, the material interacting with the exogenous source has significant absorption at a NIR wavelength in the range from 750 nm to 950 nm. In some embodiments, irradiating the particle comprises an irradiation wavelength of 780 nm to 810 nm.

In some embodiments, the material interacting with the exogenous source has significant absorption at a NIR wavelength in the range from 800 nm to 1100 nm. In some embodiments, the material interacting with the exogenous source has significant absorption at a NIR wavelength in the range from 750 nm to 850 nm. In some embodiments, the material interacting with the exogenous source has significant absorption at a NIR wavelength in the range from 1000 nm to 1400 nm. In some embodiments, the material interacting with the exogenous source has significant absorption at a NIR wavelength in the range from 1000 nm to 1300 nm. In some embodiments, the material interacting with the exogenous source has significant absorption at a NIR wavelength in the range from 1000 nm to 1100 nm. In some embodiments, the material interacting with the exogenous source has significant absorption at a wavelength selected from the group of 750 nm, 751 nm, 752 nm, 753 nm, 754 nm, 755 nm, 756 nm, 757 nm, 756 nm, 756 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 789 nm, 790 nm, 791 nm, 792 nm, 793 nm, 794 nm, 795 nm, 796 nm, 797 nm, 798 nm, 799 nm, 800 nm, 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 n, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1000 nm, 1001 nm, 1002 nm, 1003 nm, 1004 nm, 1005 nm, 1006 nm, 1007 nm, 1008 nm, 1009 nm, 1010 nm, 1011 nm, 1012 nm, 1013 nm, 1014 nm, 1015 nm, 1016 nm, 1017 nm, 1018 nm, 1019 nm, 1020 nm, 1021 nm, 1022 nm, 1023 nm, 1024 nm, 1025 nm, 1026 nm, 1027 nm, 1028 nm, 1029 nm, 1030 nm, 1031 nm, 1032 nm, 1033 nm, 1034 nm, 1035 nm, 1036 nm, 1037 nm, 1038 nm, 1039 nm, 1040 nm, 1041 nm, 1042 nm, 1043 nm, 1044 nm, 1045 nm, 1046 nm, 1047 nm, 1048 nm, 1049 nm, 1050 nm, 1051 nm, 1052 nm, 1053 nm, 1054 nm, 1055 nm, 1056 nm, 1057 nm, 1058 nm, 1059 nm, 1060 nm, 1061 nm, 1062 nm, 1063 nm, 1064 nm, 1065 nm, 1066 nm, 1067 nm, 1068 nm, 1069 nm, 1070 nm, 1071 nm, 1072 nm, 1073 nm, 1074 nm, 1075 nm, 1076 nm, 1077 nm, 1078 nm, 1079 nm, 1080 nm, 1081 nm, 1082 nm, 1083 nm, 1084 nm, 1085 nm, 1086 nm, 1087 nm, 1088 nm, 1089 nm, 1090 nm, 1091 nm, 1092 nm, 1093 nm, 1094 nm, 1095 nm, 1096 nm, 1097 nm, 1098 nm, 1099 nm, and 1100 nm. In some embodiments, the material interacting with the exogenous source has significant absorption at a wavelength selected from the group of 700 nm, 766 nm, 777 nm, 780 nm, 783 nm, 785 nm, 800 nm, 805 nm, 808 nm, 810 nm, 820 nm, 825 nm, 900 nm, 948 nm, 950 nm, 960 nm, 980 nm, 1000 nm, 1064 nm, 1065 nm, 1070 nm, 1071 nm, 1073 nm, 1098 nm, and 1100 nm.

In some embodiments, the material interacting with the exogenous source has significant absorption at 805 nm wavelength. In some embodiments, the material interacting with the exogenous source has significant absorption at 808 nm wavelength. In some embodiments, the material interacting with the exogenous source has significant absorption at 1064 nm wavelength.

In some embodiments, the material interacting with exogenous source is an infrared radiation (IR) absorbing material. In some embodiments, the IR absorbing agent comprises organic dyes or inorganic pigments. In some embodiments, the IR absorbing agent is an aminium and/or di-imonium dye having hexafluoroantimonate, tetrafluoroborate, or hexafluorophosphate as counterion. In some embodiments, an IR absorbing agent, N,N,N,N-tetrakis(4-dibutylaminophenyl)-p-benzoquinone bis(iminium hexafluoroantimonate), commercially available as ADS1065 from American Dye Source, Inc., may be utilized. The absorption spectrum of ADS1065 dye has a maximum absorption at about 1065 nm, with low absorption in the visible region of the spectrum.

In some embodiments, the material is an IR absorbing organic dye such as those Epolight™ aminium dyes made by Epolin Inc. of Newark, N.J. In some embodiments, the IR absorbing agent is an di-imonium dye (also aminium dye) having formula (I)

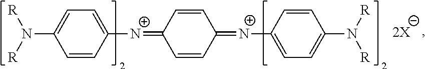

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from the group of hexafluoroarsenate (AsF₆⁻), hexafluoroantimonate (SbF₆⁻), hexafluorophosphate (PF₆⁻), tetrakis(perfluorophenyl)borate (C₆F₅)₄B⁻, and tetrafluoroborate (BF₄⁻). In some embodiments, the di-imonium dye of formula (I) has hexafluorophosphate as counterion. In some embodiments, the di-imonium dye of formula (I) has hexafluoroantimonate as counterion. In some embodiments, the di-imonium dye of formula (I) has tetrakis(perfluorophenyl)borate as counterion. In some embodiments, the IR absorbing agent is a tetrakis aminium dye, with a counterion containing metal element such as boron or antimony. In some embodiments, the tetrakis aminium dye compounds have formula (II)

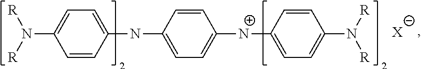

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from hexafluoroarsenate (AsF₆⁻), hexafluoroantimonate (SbF₆⁻), hexafluorophosphate (PF₆⁻), (C6F5)4B⁻, or tetrafluoroborate (BF4⁻). In some embodiments, the tetrakis aminium dyes are narrow band absorbers including commercially available dyes sold under the trademark names Epolight™ 1117 (tetrakis aminium dye having hexafluorophosphate counterion, peak absorption, 1071 nm), Epolight™ 1151 (tetrakis aminium dye, peak absorption, 1070 nm), or Epolight™ 1178 (tetrakis aminium dye, peak absorption, 1073 nm). Epolight™ 1151 (tetrakis aminium dye, peak absorption, 1070 nm), or Epolight™ 1178 (tetrakis aminium dye, peak absorption, 1073 nm). In some embodiments, the tetrakis aminium dyes are broad band absorbers including commercially available dyes sold under the trademark names Epolight™ 1175 (tetrakis aminium dye, peak absorption, 948 nm), Epolight™ 1125 (tetrakis aminium dye, peak absorption, 950 nm), and Epolight™ 1130 (tetrakis aminium dye, peak absorption, 960 nm).

In some embodiments, the material is selected from the group of a tetrakis aminium dye, a cyanine dye, a squarylium dye, indocyanine green (ICG), new ICG (IR 820), squaraine dye, IR 780 dye, IR 193 dye, Epolight™ 1117, Epolight™ 1175, iron oxide, zinc iron phosphate pigment, and combinations thereof.

In some embodiments, the infrared radiation absorbing material is a tetrakis aminium dye. In some embodiments, the tetrakis aminium dye is a narrow band absorber including commercially available dyes sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broadband absorber including commercially available dyes sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the tetrakis aminium dye is Epolight™ 1178 made by Epolin. In some embodiments, the IR absorbing agent is a tetrakis aminium dye has minimal visible color. In some embodiments, the tetrakis aminium dye is Epolight™ 1117 (molecular weight, 1211 Da, peak absorption 1098 nm).

Other suitable aminium and/or di-imonium dyes suitable for the invention in this disclosure may be found in U.S. Pat. Nos. 3,440,257, 3,484,467, 3,400,156, 5,686,639, all of which are hereby fully incorporated by reference herein in their entirety. Additional counterions for the aminium and/or di-imonium dyes may be found in U.S. Pat. No. 7,498,123, which is hereby fully incorporated by reference herein in its entirety.

In some embodiments, the material is an infrared radiation absorbing material selected from the group of 1-butyl-2-(2-[3-[2-(1-butyl-1H-benzo[cd]indol-2-ylidene)-ethylidene]-2-chloro-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclopent-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-diphenylamino-cyclopent-1-enyl]vinyl)-benzo[cd]indolium tetrafluoroborate, 1-Butyl-2-[2-[3[(1-butyl-6-chlorobenz[cd]indol-2(1H)-ylidene)ethylidene]-2-chloro-1-cyclohexen-1-yl]ethenyl]-6-chlorobenz[cd]indolium tetrafluoroborate (IR 1048), 1-butyl-2-[2-[3-[(1-butyl-6-chlorobenz[cd]indol-2(1H)-ylidene)ethylidene]-2-chloro-5-methyl-1-cyclohexen-1-yl]ethenyl]-6-chlorobenz[cd]indolium tetrafluoroborate (LumogenTM IR 1050 by BASF), 4-[2-[2-chloro-3-[(2,6-diphenyl-4H-thiopyran-4-ylidene)ethylidene]-1-cyclohexen-1-yl]ethenyl]-2,6-diphenylthiopyrylium tetrafluoroborate (IR 1061), dimethyl{4-[1,7,7-tris(4-dimethylaminophenyl)-2,4,6-heptatrienylidene]-2,5-cyclohexadien-1-ylidene}ammonium perchlorate (IR 895), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), 4-hydroxybenzoic acid appended heptamethine cyanine, amine functionalized heptamethine cyanine, hemicyanine rhodamine, cryptocyanine, diketopyrrolopyrole, diketopyrrolopyrole-croconaine, 1,3-bis(5-(ethyl(2-(prop-2-yn-1-yloxy)ethyl)amino)thiophen-2-yl)-4,5-dioxocyclopent-2-en-1-ylium-2-olate (diaminothiophene-croconaine dye), potassium 1,1′4(2-oxido-4,5-dioxocyclopent-2-en-1-ylium-1,3-diyl)bis(thiophene-5,2-diyl))bis(piperidine-4-carboxylate) (dipiperidylthiophene-croconaine dye), indocyanine green (ICG), Cyanine 7 (Cy7®), and combinations thereof.

In some embodiments, the squarylium dye is a benzopyrylium squarylium dyes having formula (III)

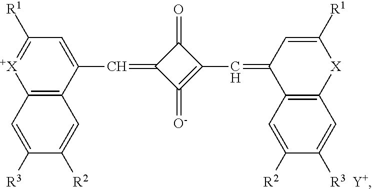

wherein each X is independently 0, S, Se; Y⁺ is a counterion selected from the group of hexafluoroarsenate (AsF₆⁻), hexafluoroantimonate (SbF₆⁻), hexafluorophosphate (PF₆⁻), (C₆F₅)₄B⁻, and tetrafluoroborate (BF₄⁻); each R¹ is a non-aromatic organic substituent, each R²═H or OR³, R³=cycloalkyl, alkenyl, acyl, silyl; each R³=—NR⁴R⁵, each R⁴, R⁵ is independently H, C1-8 alkyl. In some embodiments, the squarylium dye of formula (III) is a compound when R¹=-CMe3, R²═OCHMeEt, X═O with a strong absorption at 788 nm. In some embodiments, the squarylium dye of formula (III) is a compound when R¹=-CMe₃, R²=H, R³=-NEt₁, X═O with a strong absorption at 808 nm (IR 193 dye).

In some embodiments, the IR absorbing agent comprises cyanine dyes selected from the group indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof. In some embodiments, the infrared radiation absorbing material may include indocyanine green (ICG).

In some embodiments, the infrared radiation absorbing material may include a squarylium dye. In some embodiments, the IR absorbing agent may include squaraine dye. In some embodiments, the IR absorbing agent may include a squarylium dye selected from the group of IR 193 dye, 1,3-bis[[2-(1,1-dimethylethyl)-4H-1-benzopyran-4-ylidene]methyl]-2,4-dihydroxy-cyclobutenediylium salt, 1,3-dihydroxy-2,4-bis[(2-phenyl-4H-1-benzopyran-4-ylidene)methyl]-cyclobutenediylium salt, 1,3-bis[[2-(1,1-dimethylethyl)-6-methyl-4H-1-benzopyran-4-ylidene]methyl]-2,4-dihydroxy-cyclobutenediylium salt, 1,3-bis[[2-(1,1-dimethylethyl)-7-hydroxy-4H-1-benzopyran-4-ylidene]methyl]-2,4-dihydroxy-cyclobutenediylium salt, 1,3-bis[[2-(1,1-dimethylethyl)-6-(1-methylethyl)-4H-1-benzopyran-4-ylidene]methyl]-2,4-dihydroxy-cyclobutenediylium salt, 1,3-dihydroxy-2,4-bis[1-(2-phenyl-4H-1-benzopyran-4-ylidene)ethyl]-cyclobutenediylium salt, 1,3-dihydroxy-2,4-bis[(2-phenyl-4H-naphtho[1,2-b]pyran-4-ylidene)methyl]-cyclobutenediylium salt, 1,3-dihydroxy-2,4-bis[[6-(1-methylethyl)-2-phenyl-4H-1-benzopyran-4-ylidene]methyl]-cyclobutenediylium salt, 1,3-bis[[6-(1,1-dimethylethyl)-2-phenyl-4H-1-benzopyran-4-ylidene]methyl]-2,4-dihydroxy-cyclobutenediylium salt, 1,3-bis[(2-cyclohexyl-7-methoxy-4H-1-benzopyran-4-ylidene)methyl]-2,4-dihydroxy-cyclobutenediylium salt, 1,3-bis[[2-(1,1-dimethylethyl)-6-(1-methylpropoxy)-4H-1-benzopyran-4-ylidene]methyl]-2,4-dihydroxy-cyclobutenediylium salt, 1,3-bis[[8-chloro-2-(1,1-dimethylethyl)-6-(1-methylethyl)-4H-1-benzopyran-4-ylidene]methyl]-2,4-dihydroxy-cyclobutenediylium salt, 1,3-bis[[7-(dimethylamino)-2-(1,1-dimethylethyl)-4H-1-benzopyran-4-ylidene]methyl]-2,4-dihydroxy-cyclobutenediylium salt, 1-[[7-(diethylamino)-2-(1,1-dimethylethyl)-4H-1-benzopyran-4-ylidene]methyl]-3-[[7-(dimethylamino)-2-(1,1-dimethylethyl)-4H-1-benzopyran-4-ylidene]methyl]-2,4-dihydroxy-cyclobutenediylium salt, 1,3-bis[[7-(diethylamino)-2-(1,1-dimethylethyl)-4H-1-benzopyran-4-ylidene]methyl]-2,4-dihydroxy-cyclobutenediylium salt, 1,3-bis[1-[7-(diethylamino)-2-(1,1-dimethylethyl)-4H-1-benzopyran-4-ylidene]ethyl]-2,4-dihydroxy-cyclobutenediylium salt, 1-[[7-(diethylamino)-2-(1,1-dimethylethyl)-4H-1-benzopyran-4-ylidene]methyl]-3-[[2-(1,1-dimethylethyl)-7-(2-ethylbutoxy)-4H-1-benzopyran-4-ylidene]methyl]-2,4-dihydroxy-cyclobutenediylium salt, 1,3-bis[[2-cyclohexyl-7-(diethylamino)-4H-1-benzopyran-4-ylidene]methyl]-2,4-dihydroxy-cyclobutenediylium salt, 1,3-bis[[2-(1,1-dimethylethyl)-7-(1-piperidinyl)-4H-1-benzopyran-4-ylidene]methyl]-2,4-dihydroxy-cyclobutenediylium salt, 1,3-bis[[2-(1,1-dimethylethyl)-7-(hexahydro-1H-azepin-1-yl)-4H-1-benzopyran-4-ylidene]methyl]-2,4-dihydroxy-cyclobutenediylium salt, 1,3-bis[[2-(1,1-dimethylethyl)-7-(4-morpholinyl)-4H-1-benzopyran-4-ylidene]methyl]-2,4-dihydroxy-cyclobutenediylium salt, 1,3-bis[[11-(1,1-dimethylethyl)-2,3,6,7-tetrahydro-1H,5H,9H-[1]benzopyrano[6,7,8-ij]quinolizin-9-ylidene]methyl]-2,4-dihydroxy-cyclobutenediylium salt, 1,3-bis[[2-(1,1-dimethylethyl)-6-(4-morpholinyl)-4H-1-benzopyran-4-ylidene]methyl]-2,4-dihydroxy-cyclobutenediylium salt, 1,3-bis[[2-bicyclo[2.2.1]hept-5-en-2-yl-7-(diethylamino)-4H-1-benzopyran-4-ylidene]methyl]-2,4-dihydroxy- cyclobutenediylium salt, 1,3 -bis [[7-(2,3 -dihydro-1Hindol-1-yl)-2-(1,1-dimethylethyl)-4H-1-benzopyran-4-ylidene]methyl]-2,4-dihydroxy-cyclobutenediylium salt, 1,3-bis[[7-(diethylamino)-2-[(1R,5S)-6,6-dimethylbicyclo[3.1.1]hept-2-en-2-yl]-4H-1-benzopyran-4-ylidene]methyl]-2,4-dihydroxy-cyclobutenediylium salt, 1,3-bis[[7-(diethylamino)-2-(6,6-dimethylbicyclo[3.1.1]hept-2-en-3-yl)-4H-1-benzopyran-4-ylidene]methyl]-2,4-dihydroxy-cyclobutenediylium salt, 1,3-dihydroxy-2,4-bis[[7-(4-morpholinyl)-2-tricyclo[3.3.1.13,7]dec-1-yl-4H-1-benzopyran-4-ylidene]methyl]-cyclobutenediylium salt, 2,4-bis[[7-(diethylamino)-2-(1,1-dimethylethyl)-4H-1-benzopyran-4-ylidene]methyl]-1,3-cyclobutanedione, and combinations thereof.

In some embodiments, the material is an infrared radiation absorbing agent selected from the group of phthalocyanines, naphthalocyanines, and combinations thereof. In some embodiments, the material is selected from the group of a tri-aminium dye, a tetrakis aminium dye, a cyanine dye, a squarylium dye, an inorganic infrared radiation absorbing material, and combinations thereof. In some embodiments, the material is a squaraine dye. In some embodiments, the material is a tetrakis aminium dye. In some embodiments, the material is a squarylium dye. In some embodiments, the material is an inorganic IR absorbing agent. In some embodiments, the IR absorbing agent is an organic IR absorbing agent. In some embodiments, the IR absorbing agent is an aminium and/or di-imonium dye having hexafluoroantimonate, tetrafluoroborate, or hexafluorophosphate as counterion. In some embodiments, an infrared radiation absorbing material, N,N,N,N-tetrakis(4-dibutylaminophenyl)-p-benzoquinone bis(iminium hexafluoroantimonate), commercially available as ADS1065 from American Dye Source, Inc., may be utilized. The absorption spectrum of ADS1065 dye has a maximum absorption at about 1065 nm, with low absorption in the visible region of the spectrum. In some embodiments, the IR absorbing agent is indocyanine green (ICG) or new ICG dye IR820.

In some embodiments, the infrared-absorbing materials are inorganic substances that contain specific chemical elements having an incomplete electronic d-shell (i.e. atoms or ions of transition elements), and whose infrared absorption is a consequence of electronic transitions within the d-shell of the atom or ion. In some embodiments, the inorganic infrared radiation absorbing materials comprise one or more transition metal elements in the form of an ion such as a palladium(II), a platinum(II), a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the materials are inorganic infrared radiation absorbing materials with near-infrared absorbing properties selected from the group of iron oxide nanoparticle, zinc copper phosphate pigment ((Zn,Cu)2P207), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing agent is a zinc iron phosphate pigment. In some embodiments, the inorganic IR absorbing agent may include palladate (e.g. barium tetrakis(cyano-C)palladate tetrahydrate, BaPd(CN)₄.4H₂O, [Pd(dimit)2]²⁻, bis(1,3-dithiole-2-thione-4,5-dithiolate)palladate(II). In some embodiments, the inorganic infrared radiation absorbing material may include platinate, e.g. platinum-based polypyridyl complexes with dithiolate ligands, Pt(II)(diamine)(dithiolate) with 3,3′-, 4,4′-, 5,5′-bipyridyl substituents.

In some embodiments, the infrared radiation absorbing material is admixed within the carrier to form a homogeneous dispersion or a solid solution. In some embodiments, the infrared radiation absorbing material and the carrier may have oppositely charged functional group(s) (e.g. infrared radiation absorbing material is positively charged tetrakis aminium dye, and the carrier has negatively charged functional group such as carboxylate anion of polymethacrylate polymers) such that the infrared radiation absorbing material attaches to the carrier via hydrogen bond or via ionic electrostatic interactions.

In some embodiments, the particle exhibits energy-to-heat conversion stability such that the loss in absorbance of the infrared radiation absorbing material is less than 50% as measured by the Material Process Stability Test after exposure to a pulsed laser light.

The preferred concentration of the material responsive to the exogenous source is dependent on the amount required to obtain the desired response to the source. For example, in the case of an IR absorbing agent needed to absorb incident infrared radiation, then too little dye can limit the temperature rise that would be desired. Likewise, too high a concentration can lead to dye aggregation, which can shift the absorption, such that the dye no longer absorbs the wavelength provided by the laser. In some embodiments, the material responsive to the exogenous source is present in an amount ranging from about 0.01 wt. % to about 25.0 wt. % by the total weight of the particle. In some embodiments, the material responsive to the exogenous source is present in an amount ranging from about 1.0 wt. % to about 20.0 wt. % by the total weight of the particle. In some embodiments, the material responsive to the exogenous source is present in an amount ranging from about 5.0 wt. % to about 20.0 wt. % by the total weight of the particle. In some embodiments, the material responsive to the exogenous source is present in an amount ranging from about 5.0 wt. % to about 15.0 wt. % by the total weight of the particle. In some embodiments, the material responsive to the exogenous source is present in an amount ranging from about 5.5 wt. % to about 15.0 wt. % by the total weight of the particle. In some embodiments, the material responsive to the exogenous source is present in an amount ranging from about 6.0 wt. % to about 15.0 wt. % by the total weight of the particle. In some embodiments, the material responsive to the exogenous source is present in an amount ranging from about 6.5 wt. % to about 15.0 wt. % by the total weight of the particle. In some embodiments, the material responsive to the exogenous source is present in an amount ranging from about 7.0 wt. % to about 15.0 wt. % by the total weight of the particle. In some embodiments, the material responsive to the exogenous source is present in an amount ranging from about 7.5 wt. % to about 15.0 wt. % by the total weight of the particle. In some embodiments, the material responsive to the exogenous source is present in an amount ranging from about 8.0 wt. % to about 15.0 wt. % by the total weight of the particle. In some embodiments, the material responsive to the exogenous source is present in an amount ranging from about 8.5 wt. % to about 15.0 wt. % by the total weight of the particle. In some embodiments, the material responsive to the exogenous source is present in an amount ranging from about 9.0 wt. % to about 15.0 wt. % by the total weight of the particle. In some embodiments, the material responsive to the exogenous source is present in an amount ranging from about 9.5 wt. % to about 15.0 wt. % by the total weight of the particle. In some embodiments, the material responsive to the exogenous source is present in an amount ranging from about 10.0 wt. % to about 15.0 wt. % by the total weight of the particle. In some embodiments, the material responsive to the exogenous source is present in an amount ranging from about 10.5 wt. % to about 15.0 wt. % by the total weight of the particle. In some embodiments, the material responsive to the exogenous source is present in an amount ranging from about 11.0 wt. % to about 15.0 wt. % by the total weight of the particle. In some embodiments, the material responsive to the exogenous source is present in an amount ranging from about 11.5 wt. % to about 15.0 wt. % by the total weight of the particle. In some embodiments, the material responsive to the exogenous source is present in an amount ranging from about 12.0 wt. % to about 15.0 wt. % by the total weight of the particle. In some embodiments, the material responsive to the exogenous source is present in an amount ranging from about 12.5 wt. % to about 15.0 wt. % by the total weight of the particle. In some embodiments, the material responsive to the exogenous source is present in an amount ranging from about 13.0 wt. % to about 15.0 wt. % by the total weight of the particle. In some embodiments, the material responsive to the exogenous source is present in an amount ranging from about 13.5 wt. % to about 15.0 wt. % by the total weight of the particle. In some embodiments, the material responsive to the exogenous source is present in an amount ranging from about 14.0 wt. % to about 15.0 wt. % by the total weight of the particle. In some embodiments, the material responsive to the exogenous source is present in an amount ranging from about 5.0 wt. % to about 14.0 wt. % by the total weight of the particle. In some embodiments, the material responsive to the exogenous source is present in an amount ranging from about 5.5 wt. % to about 14.0 wt. % by the total weight of the particle. In some embodiments, the material responsive to the exogenous source is present in an amount ranging from about 6.0 wt. % to about 14.0 wt. % by the total weight of the particle. In some embodiments, the material responsive to the exogenous source is present in an amount ranging from about 6.5 wt. % to about 14.0 wt. % by the total weight of the particle. In some embodiments, the material responsive to the exogenous source is present in an amount ranging from about 7.0 wt. % to about 14.0 wt. % by the total weight of the particle. In some embodiments, the material responsive to the exogenous source is present in an amount ranging from about 7.5 wt. % to about 14.0 wt. % by the total weight of the particle. In some embodiments, the material responsive to the exogenous source is present in an amount ranging from about 8.0 wt. % to about 14.0 wt. % by the total weight of the particle. In some embodiments, the material responsive to the exogenous source is present in an amount ranging from about 8.5 wt. % to about 14.0 wt. % by the total weight of the particle. In some embodiments, the material responsive to the exogenous source is present in an amount ranging from about 9.0 wt. % to about 14.0 wt. % by the total weight of the particle. In some embodiments, the material responsive to the exogenous source is present in an amount ranging from about 9.5 wt. % to about 14.0 wt. % by the total weight of the particle. In some embodiments, the material responsive to the exogenous source is present in an amount ranging from about 10.0 wt. % to about 14.0 wt. % by the total weight of the particle. In some embodiments, the material responsive to the exogenous source is present in an amount ranging from about 10.5 wt. % to about 14.0 wt. % by the total weight of the particle. In some embodiments, the material responsive to the exogenous source is present in an amount ranging from about 11.0 wt. % to about 14.0 wt. % by the total weight of the particle. In some embodiments, the material responsive to the exogenous source is present in an amount ranging from about 11.5 wt. % to about 14.0 wt. % by the total weight of the particle. In some embodiments, the material responsive to the exogenous source is present in an amount ranging from about 12.0 wt. % to about 14.0 wt. % by the total weight of the particle. In some embodiments, the material responsive to the exogenous source is present in an amount ranging from about 12.5 wt. % to about 14.0 wt. % by the total weight of the particle. In some embodiments, the material responsive to the exogenous source is present in an amount ranging from about 13.0 wt. % to about 14.0 wt. % by the total weight of the particle. In some embodiments, the material responsive to the exogenous source is present in an amount ranging from about 13.5 wt. % to about 14.0 wt. % by the total weight of the particle. In some embodiments, the material responsive to the exogenous source is present in an amount ranging from about 5.0 wt. % to about 13.0 wt. % by the total weight of the particle. In some embodiments, the material responsive to the exogenous source is present in an amount ranging from about 5.5 wt. % to about 13.0 wt. % by the total weight of the particle. In some embodiments, the material responsive to the exogenous source is present in an amount ranging from about 6.0 wt. % to about 13.0 wt. % by the total weight of the particle. In some embodiments, the material responsive to the exogenous source is present in an amount ranging from about 6.5 wt. % to about 13.0 wt. % by the total weight of the particle. In some embodiments, the material responsive to the exogenous source is present in an amount ranging from about 7.0 wt. % to about 13.0 wt. % by the total weight of the particle. In some embodiments, the material responsive to the exogenous source is present in an amount ranging from about 7.5 wt. % to about 13.0 wt. % by the total weight of the particle. In some embodiments, the material responsive to the exogenous source is present in an amount ranging from about 8.0 wt. % to about 13.0 wt. % by the total weight of the particle. In some embodiments, the material responsive to the exogenous source is present in an amount ranging from about 8.5 wt. % to about 13.0 wt. % by the total weight of the particle. In some embodiments, the material responsive to the exogenous source is present in an amount ranging from about 9.0 wt. % to about 13.0 wt. % by the total weight of the particle. In some embodiments, the material responsive to the exogenous source is present in an amount ranging from about 9.5 wt. % to about 13.0 wt. % by the total weight of the particle. In some embodiments, the material responsive to the exogenous source is present in an amount ranging from about 10.0 wt. % to about 13.0 wt. % by the total weight of the particle. In some embodiments, the material responsive to the exogenous source is present in an amount ranging from about 10.5 wt. % to about 13.0 wt. % by the total weight of the particle. In some embodiments, the material responsive to the exogenous source is present in an amount ranging from about 11.0 wt. % to about 13.0 wt. % by the total weight of the particle. In some embodiments, the material responsive to the exogenous source is present in an amount ranging from about 11.5 wt. % to about 13.0 wt. % by the total weight of the particle. In some embodiments, the material responsive to the exogenous source is present in an amount ranging from about 12.0 wt. % to about 13.0 wt. % by the total weight of the particle.

In some embodiments, the material responsive to the exogenous source is present in an amount ranging from about 5.0 wt. % to about 12.0 wt. % by the total weight of the particle. In some embodiments, the material responsive to the exogenous source is present in an amount ranging from about 5.5 wt. % to about 12.0 wt. % by the total weight of the particle. In some embodiments, the material responsive to the exogenous source is present in an amount ranging from about 6.0 wt. % to about 12.0 wt. % by the total weight of the particle. In some embodiments, the material responsive to the exogenous source is present in an amount ranging from about 6.5 wt. % to about 12.0 wt. % by the total weight of the particle. In some embodiments, the material responsive to the exogenous source is present in an amount ranging from about 7.0 wt. % to about 12.0 wt. % by the total weight of the particle. In some embodiments, the material responsive to the exogenous source is present in an amount ranging from about 7.5 wt. % to about 12.0 wt. % by the total weight of the particle. In some embodiments, the material responsive to the exogenous source is present in an amount ranging from about 8.0 wt. % to about 12.0 wt. % by the total weight of the particle. In some embodiments, the material responsive to the exogenous source is present in an amount ranging from about 8.5 wt. % to about 12.0 wt. % by the total weight of the particle. In some embodiments, the material responsive to the exogenous source is present in an amount ranging from about 9.0 wt. % to about 12.0 wt. % by the total weight of the particle. In some embodiments, the material responsive to the exogenous source is present in an amount ranging from about 9.5 wt. % to about 12.0 wt. % by the total weight of the particle. In some embodiments, the material responsive to the exogenous source is present in an amount ranging from about 10.0 wt. % to about 12.0 wt. % by the total weight of the particle. In some embodiments, the material responsive to the exogenous source is present in an amount ranging from about 10.5 wt. % to about 12.0 wt. % by the total weight of the particle. In some embodiments, the material responsive to the exogenous source is present in an amount ranging from about 11.0 wt. % to about 12.0 wt. % by the total weight of the particle. In some embodiments, the material responsive to the exogenous source is present in an amount ranging from about 11.5 wt. % to about 12.0 wt. % by the total weight of the particle.

In some embodiments, the material responsive to the exogenous source is present in an amount ranging from about 5.0 wt. % to about 11.0 wt. % by the total weight of the particle. In some embodiments, the material responsive to the exogenous source is present in an amount ranging from about 5.5 wt. % to about 11.0 wt. % by the total weight of the particle. In some embodiments, the material responsive to the exogenous source is present in an amount ranging from about 6.0 wt. % to about 11.0 wt. % by the total weight of the particle. In some embodiments, the material responsive to the exogenous source is present in an amount ranging from about 6.5 wt. % to about 11.0 wt. % by the total weight of the particle. In some embodiments, the material responsive to the exogenous source is present in an amount ranging from about 7.0 wt. % to about 11.0 wt. % by the total weight of the particle. In some embodiments, the material responsive to the exogenous source is present in an amount ranging from about 7.5 wt. % to about 11.0 wt. % by the total weight of the particle. In some embodiments, the material responsive to the exogenous source is present in an amount ranging from about 8.0 wt. % to about 11.0 wt. % by the total weight of the particle. In some embodiments, the material responsive to the exogenous source is present in an amount ranging from about 8.5 wt. % to about 11.0 wt. % by the total weight of the particle. In some embodiments, the material responsive to the exogenous source is present in an amount ranging from about 9.0 wt. % to about 11.0 wt. % by the total weight of the particle. In some embodiments, the material responsive to the exogenous source is present in an amount ranging from about 9.5 wt. % to about 11.0 wt. % by the total weight of the particle. In some embodiments, the material responsive to the exogenous source is present in an amount ranging from about 10.0 wt. % to about 11.0 wt. % by the total weight of the particle. In some embodiments, the material responsive to the exogenous source is present in an amount ranging from about 10.5 wt. % to about 11.0 wt. % by the total weight of the particle.

In some embodiments, the material responsive to the exogenous source is present in an amount ranging from about 5.0 wt. % to about 10.0 wt. % by the total weight of the particle. In some embodiments, the material responsive to the exogenous source is present in an amount ranging from about 5.5 wt. % to about 10.0 wt. % by the total weight of the particle. In some embodiments, the material responsive to the exogenous source is present in an amount ranging from about 6.0 wt. % to about 10.0 wt. % by the total weight of the particle. In some embodiments, the material responsive to the exogenous source is present in an amount ranging from about 6.5 wt. % to about 10.0 wt. % by the total weight of the particle. In some embodiments, the material responsive to the exogenous source is present in an amount ranging from about 7.0 wt. % to about 10.0 wt. % by the total weight of the particle. In some embodiments, the material responsive to the exogenous source is present in an amount ranging from about 7.5 wt. % to about 10.0 wt. % by the total weight of the particle. In some embodiments, the material responsive to the exogenous source is present in an amount ranging from about 8.0 wt. % to about 10.0 wt. % by the total weight of the particle. In some embodiments, the material responsive to the exogenous source is present in an amount ranging from about 8.5 wt. % to about 10.0 wt. % by the total weight of the particle. In some embodiments, the material responsive to the exogenous source is present in an amount ranging from about 9.0 wt. % to about 10.0 wt. % by the total weight of the particle.

In some embodiments, the material responsive to the exogenous source is present in an amount ranging from about 8.0 wt. % to about 10.0 wt. % by the total weight of the particle. In some embodiments, the material responsive to the exogenous source is present in an amount ranging from about 7.0 wt. % to about 10.0 wt. % by the total weight of the particle. In some embodiments, the material responsive to the exogenous source is present in an amount ranging from about 6.0 wt. % to about 10.0 wt. % by the total weight of the particle. In some embodiments, the material responsive to the exogenous source is present in an amount ranging from about 5.0 wt. % to about 10.0 wt. % by the total weight of the particle. In some embodiments, the material responsive to the exogenous source is present in an amount ranging from about 4.0 wt. % to about 10.0 wt. % by the total weight of the particle. In some embodiments, the material responsive to the exogenous source is present in an amount ranging from about 3.0 wt. % to about 10.0 wt. % by the total weight of the particle. In some embodiments, the material responsive to the exogenous source is present in an amount selected from the group of: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. %. In some embodiments, the material responsive to the exogenous source is present in an amount selected from the group of: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. %. In some embodiments, the material responsive to the exogenous source is present in an amount selected from the group of: about 1.0 wt. %, about 5.0 wt. %, about 10.0 wt. %, and about 15.0 wt. %.

In some embodiments, the particle has a ratio of the weight amount of the material responsive to the exogenous source to the colorant of 10:1 to 1:10. In some embodiment, the ratio of the weight amount of the material responsive to the exogenous source to the colorant is 10:1, 9:1, 8:1, 7:1, 6:1, 5:1, 4:1, 3:1, 2:1, 1:1, 1;2, 1:3, 1:4, 1:5, 1:6, 1:7, 1:8, 1:9, or 1:10. In some embodiments, the ratio of the weight amount of the material responsive to the exogenous source to the colorant is 1:1.

In some embodiments, the tattoo particle exhibits stability such that the degradation of the material by body chemicals is less than 20% as measured by the Efficacy Determination Protocol after incubating the particles in the extraction medium (containing serum) for 24 hours at 37° C. In some embodiments, the tattoo particle exhibits stability such that the colorant has a degree of degradation selected from the group of about 5.0%, about 10%, about 15%, about 20% as measured by Efficacy Determination Protocol. In some embodiments, the colorant and/or the material has a degree of degradation in a range selected from the group of less than about 20.0%, less than about 15.0%, less than about 10.0%, less than about 5.0%, less than about 1.0% , less than about 0.5%, less than about 0.1%, and less than about 0.01% as determined by Efficacy Determination Protocol. In some embodiments, the colorant and/or the material has a degree of degradation less than about 10.0% as determined by Efficacy Determination Protocol. In some embodiments, the colorant and/or the material has a degree of degradation less than about 5.0% as measured by Efficacy Determination Protocol. In some embodiments, the colorant and/or the material has a degree of degradation less than about 1.0% as measured by Efficacy Determination Protocol. In some embodiments, the colorant and/or the material responsive to exogenous source has a degree of degradation less than about 0. 1% as measured by Efficacy Determination Protocol.

### (d) Optional Additives

In some embodiments, the core of the particle may optionally comprise an additive. In some embodiments, the additive is a thermal stabilizer, an antioxidant, or a surfactant.

In some embodiments, the particle further includes thermal stabilizers. It should be noted that often the colorant and/or the material that interact with the exogenous source can be stable (low rate of degradation) at room temperature, but when the particle comprising the colorant and the material is inside body, at body temperature of 37.5° C., degradation of the colorant and the material can be significantly accelerated. Examples of useful thermal stabilizers include phenolic antioxidants such as butylated hydroxytoluene (BHT), 2-tent-butyl-hydroquinone, and 2-tert-butyl-hydroxyanisole.

In some embodiments, the additive is an antioxidant for stabilizing the dyes. In some embodiments, the additive is an antioxidant for stabilizing the dyes at human body temperature. In some embodiments, the antioxidants for stabilizing dyes comprise sterically hindered phenols with para-propionate groups. In some embodiments, the antioxidant for stabilizing dyes comprises pentaerythritol tetrakis(3,5-di-tert-butyl-4-hydroxyhydrocinnamate). In some embodiments, the antioxidant for stabilizing dyes comprises a phosphite such as tris(2,4-di-tert-butylphenyl)phosphite. In some embodiments, the antioxidant for stabilizing dyes comprises organosulfur compounds such as thioethers. In some embodiments, the antioxidant for stabilizing dyes comprises 1,3,5-TR1S(4-tert-butyl-3-hydroxy-2,6-dimethylbenzyl)-1,3,5-triazine-2,4,6-(1H,3H,5H)-trione (Cyanox® 1790); wherein the Cyanox® 1790 is colorless.

In some embodiments, the additive is a surfactant. In some embodiment, the surfactant may include cationic, amphoteric, and non-ionic surfactants. In some embodiments, the surfactants comprise anionic surfactants selected from fatty acid salts, bile salts, phospholipids, carnitines, ether carboxylates, succinylated monoglycerides, mono/diacetylated tartaric acid esters of mono- and diglycerides, citric acid esters of mono-, diglycerides, sodium oleate, sodium lauryl sulfate, sodium lauryl sarcosinate, sodium dioctyl sulfosuccinate (SDS), sodium cholate, sodium taurocholate, lauroyl carnitine; palmitoyl carnitine; and myristoyl carnitine, or lactylic esters of fatty acids. In some embodiments, anionic surfactants include di-(2-ethylhexyl) sodium sulfosuccinate. In some embodiments, the surfactants are non-ionic surfactants selected from propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters. In some embodiments, the surfactant may comprise the fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic amphiphilic surfactant polyoxyethylene (20) sorbitan monolaurate (TWEEN® 20) or polyvinyl alcohol that improves the distribution of IR absorbing agent in the polymeric carrier. In some embodiments, the surfactant comprises an amphiphilic surfactant if the IR absorbing agent is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

In some embodiments, the use amount of the additive may be about 0.01 wt. % to about 10.0 wt. % by the total weight of the particle. In some embodiments, the use amount of the additive may be about 0.1 wt. % to about 10.0 wt. % by the total weight of the particle. In some embodiments, the use amount of the additive may be about 0.5 wt. % to about 10.0 wt. % by the total weight of the particle. In some embodiments, the use amount of the additive may be about 1.0 wt. % to about 10.0 wt. % by the total weight of the particle. In some embodiments, the use amount of the additive may be about 1.0 wt. % to about 9.0 wt. % by the total weight of the particle. In some embodiments, the use amount of the additive may be about 1.0 wt. % to about 8.0 wt. % by the total weight of the particle. In some embodiments, the use amount of the additive may be about 1.0 wt. % to about 7.0 wt. % by the total weight of the particle. In some embodiments, the use amount of the additive may be about 1.0 wt. % to about 6.0 wt. % by the total weight of the particle. In some embodiments, the use amount of the additive may be about 1.0 wt. % to about 5.0 wt. % by the total weight of the particle. In some embodiments, the use amount of the additive may be about 1.0 wt. % to about 4.0 wt. % by the total weight of the particle. In some embodiments, the use amount of the additive may be about 1.0 wt. % to about 3.0 wt. % by the total weight of the particle. In some embodiments, the use amount of the additive may be about 1.0 wt. % to about 2.5 wt. % by the total weight of the particle. In some embodiments, the use amount of the additive may be about 1.0 wt. % to about 2.0 wt. % by the total weight of the particle. In some embodiments, the use amount of the additive may be about 2.0 wt. % to about 10.0 wt. % by the total weight of the particle. In some embodiments, the use amount of the additive may be about 3.0 wt. % to about 10.0 wt. % by the total weight of the particle. In some embodiments, the use amount of the additive may be about 4.0 wt. % to about 10.0 wt. % by the total weight of the particle. In some embodiments, the use amount of the additive may be about 5.0 wt. % to about 10.0 wt. % by the total weight of the particle. In some embodiments, the use amount of the additive may be selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.25 wt. %, about 2.5 wt. %, about 2.75 wt. %, about 3.0 wt. %, about 3.25 wt. %, about 3.50 wt. %, about 3.75 wt. %, about 4.00 wt. %, about 4.25 wt. %, about 4.50 wt. %, about 4.75 wt. %, about 5.00 wt. %, about 5.25 wt. %, about 5.50 wt. %, about 5.75 wt. %, about 6.00 wt. %, about 6.25 wt. %, about 6.50 wt. %, about 6.75 wt. %, about 7.00 wt. %, about 7.25 wt. %, about 7.50 wt. %, about 7.75 wt. %, about 8.00 wt. %, about 8.25 wt. %, about 8.50 wt. %, about 8.75 wt. %, about 9.00 wt. %, about 9.25 wt. %, about 9.50 wt. %, about 9.75 wt. %, about 10.0 wt. %, about 10.25 wt. %, about 10.50 wt. %, about 10.75 wt. %, or about 11.00 wt. %.

### 2. Particle Properties

### (a) Particle Size and Morphology

In some embodiments, the particles are microparticles. In some embodiments, the particles may have spherical shape.

In some embodiments, the particles may have a wide variety of non-spherical shapes. In some embodiments, the non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non- spherical” shapes.

In some embodiments, the particles have a PdI from about 0.05 to about 0.15, about 0.06 to about 0.14, about 0.07 to about 0.13, about 0.08 to about 0.12, or about 0.09 to about 0.11. In some embodiments, the particles have a PdI of about 0.05, about 0.06, about 0.07, about 0.08, about 0.09, about 0.10, about 0.11, about 0.12, about 0.13, about 0.14, or about 0.15.

In some embodiments, the particle has a median particle size less than 1000 nm. In some embodiments, the particle has a median particle size selected from the group of about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In some embodiments, the particle has a median particle size of 500 nm. In some embodiments, the particle has a median particle size of 750 nm.

In some embodiments, the particles are microparticles having a median particle size equal or greater than 1000 nm (1 micron). In some embodiments, the particles have a median particle size selected from the group of about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm. In some embodiments, the particle has a median particle size in a range from about 1 μm to about 500 μm. In some embodiments, the particle has a median particle size in a range from about 1 μm to about 250 μm. In some embodiments, the particle has a median particle size in a range from about 1 μm to about 100 μm. In some embodiments, the particle has a median particle size in the range from about 1 μm to about 50 μm. In some embodiments, the particle has a median particle size in a range from about 1 μm to about 25 μm. In some embodiments, the particle has a median particle size distribution in a range from about 1 μm to about 10 μm. In some embodiments, the particle has a median particle size in a range from about 1 μm to about 6 μm. In some embodiments, the particle has a median particle size in a range from about 1 μm to about 5 μm. In some embodiments, the particle has a median particle size in a range from about 1 μm to about 3 μm. In some embodiments, the particle has a median particle size in a range from about 1 μm to about 2 μm. In some embodiments, the particle has a median particle size in a range from about 2 μm to about 5 μm. In some embodiments, the particle has a median particle size in a range from about 2 μm to about 4 μm. In some embodiments, the particle has a median particle size in a range from about 2 μm to about 3 μm. In some embodiments, the particle has a median particle size in a range from about 3 μm to about 5 μm. In some embodiments, the particle has a median particle size in a range from about 3 μm to about 4 μm. In some embodiments, the particle has a median particle size in a range from about 4 μm to about 5 μm. In some embodiments, the particle has a median particle size from about 1 μm, about 2 μm, about 3 μm, about 4 μm, about 5 μm, or about 6 μm. In some embodiments, the particle has a median particle size in the range from about 1 μm to about 2 μm. In some embodiments, the particle has a median particle size in the range from about 1 μm to about 3 μm. In some embodiments, the particle has a median particle size in the range from about 1 μm to about 4 μm.

### (b) Cytotoxicity and Porosity, Colorant Stability

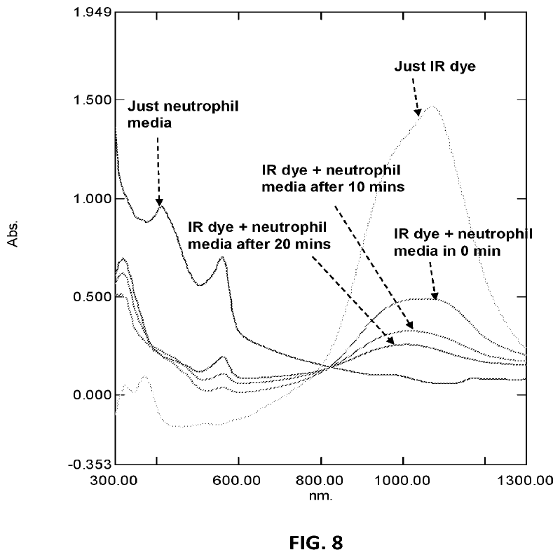

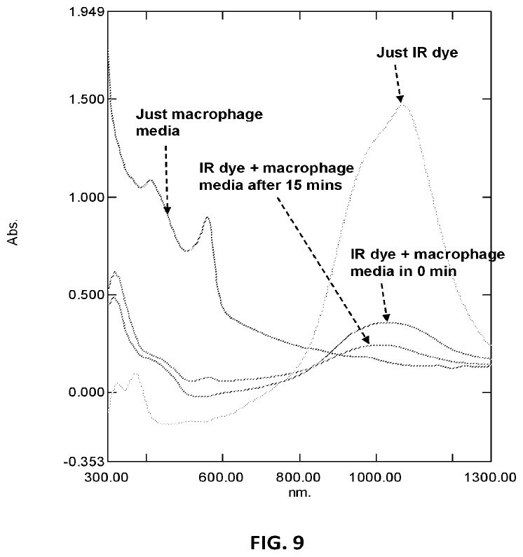

The efficacy of particles containing a colorant and a material that interacts with an exogenous source can be reduced by the leakage of the colorant and/or the material, or by incursion into the particle of the body chemicals that can degrade these components. In particular, colorants and IR absorbing agents can be susceptible to degradation by the body chemicals present in the bodily fluids, or cell growth medium such as neutrophil and macrophage media. For example, IR absorbing agent, such as Epolight™ 1117, leached from the particles, degrades when exposed to nucleophiles and free radicals (See FIGS. 8 and 9).

The encapsulated colorant and/or the material that interacts with an exogenous source within a polymeric particle may be protected from degradation by limiting their exposure to the chemistry from the surrounding environment (e.g., chemicals in the neutrophil medium or macrophage medium). However, due to the inherent porosity of the carrier of the polymeric particle, to some extent, the degrading body chemicals can still diffuse into the particle, causing the degradation of the encapsulated colorant and/or the material that interacts with an exogenous source. Further, the encapsulated colorant and/or the material that interacts with an exogenous source can also leak outside the particle, causing toxicity to the surrounding environment. Judicious choice of polymer carrier can provide some control over such incursion or leakage, but may not be enough to assure passing the Efficacy Determination Protocol or the Extractable Cytotoxicity Test. In one aspect, the disclosure provides a solution to such incursion or leakage through the use of a shell barrier to enclose the particle to reduce the particle's inherent porosity.

Thus, this disclosure provides a core-shell particle encapsulating a colorant and a material that interacts with an exogenous source which may be susceptible to degradation by the exterior degrading body chemicals such as those in the bodily fluid, wherein the shell provides additional barrier properties for limiting leakage of colorant out of the particle and into the body thereby enabling achievement of the desired cytotoxic properties. Furthermore, particles with suitable shell barrier properties can limit incursion by bodily chemicals, rendering the encapsulated colorant or material stable for a long period of time. As such, even when the carriers are porous or otherwise permit incursion by fluids and other components from the surrounding environment, the use of a well-designed shell can provide enough of a barrier to limit leakage out of and incursion into the particle so that cytotoxicity requirements can be met and particle efficacy can be maintained.

For example, to protect the IR absorbing agent encapsulated in a polymeric particle when introduced into human skin, a sol-gel vinyl-modified silicate polymer shell derived from vinyl trimethoxysilane (VTMS) is formed on the surface of the polymeric particle to block the free exchange of nucleophiles and free radical species between the particles and the surrounding environment.

In some embodiments, the free volume or the porosity associated with the polymer-based particles can be influenced by the particle fabrication process and the nature of the carrier used. The porosity or the free volume of polymeric particles having a colorant encapsulated within in a given situation is dependent upon many factors, such as branching and cross-linking of the polymer carrier, polymer crystallinity, and the dissolution of other components in the particles. Likewise, any protective shell can have some degree of porosity that depends on the conditions and materials used in its fabrication. As a result, the polymeric particles can be designed or otherwise tuned to achieve a desired amount of porosity to maintain the integrity of the contained components. In some embodiments, the disclosure provides a method of tuning particle porosity guided by the feedback loop (FIG. 1) described below to assess whether, in a specific situation, the particle construction is adequate for minimizing the leakage and also for preventing the degradation caused by the bodily fluid permeated into the interior of the particles. In an embodiment, the feedback loop is based on the Extractable Cytotoxicity Test. In an embodiment, the feedback loop is based on the Efficacy Determination Protocol. In an embodiment, the feedback loop is based on the Extractable Cytotoxicity Test and/or the Efficacy Determination Protocol.

A feedback loop (FIG. 1) based on the Extractable Cytotoxicity Test has been developed to evaluate if the particle porosity is acceptable or needs to be reduced to successfully pass the Extractable Cytotoxicity Test. If initial results are not acceptable then the particle porosity is decreased by altering the chemistry of the particle fabrication in one or more iterative steps, e.g., varying the degree of cross-linking, or adding a second carrier entrapping the first polymeric carrier, or adding a shell, or varying the shell thickness. This is done iteratively until such time as the particle passes the Extractable Cytotoxicity Test.

The details of the Extractable Cytotoxicity Test are described in Example 4. The concentration of the colorant and of other chemical components in the extract (“extract concentration”) or the dilutions thereof can be measured using analytical tools like UV-VIS-NIR, NMR, HPLC, LCMS, etc. In brief, a physiologically relevant media that contains serum proteins at physiological temperature is used to extract the enclosed colorant or the material that interacts with an exogenous source from the particles. The extract can then be used as is (“neat” or 1X) or in serial dilutions up to 10,000 times dilutions (0.0001X). In an embodiment, the dilution is selected from the group of 2, 3, 4, 5, 6, 7, 8, 9, 10, 20, 30, 40, 50 60, 70, 80, 90, 100, 150, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 times. In some embodiment, the dilution is 10 times (0.1×), 25 times (0.25×), 50 times (0.5×), 100 times (0.01×), or 200 times (0.02×). The dilution with the media in a cytotoxicity test against healthy cells depends upon the specific cells and the use of the colorant. This test is referred to as the “Extractable Cytotoxicity Test” (ECT). The neat or dilution of the extract that kills 30% of the cells can be measured and is referred to as an IC₃₀. An IC₃₀ of the particles can be established for every application. Once an IC₃₀ has been established, analysis of the concentrations of the colorant and/or the material that interacts with an exogenous source in the leachate can be used as a surrogate for the test of cytotoxicity. In the Extractable Cytotoxicity Test, in an embodiment, if the neat or dilution concentration of the colorant and/or the material that interacts with an exogenous source in the leachate is less than IC₃₀, the particle passes the Extractable Cytotoxicity Test. In some embodiments, if the neat or dilution concentration of the colorant and/or the material that interacts with an exogenous source in the leachate is less than IC₁₀, IC₂₀, IC₄₀, IC₅₀, IC₆₀, IC₇₀, IC₈₀, or IC₉₀, the particle passes the Extractable Cytotoxicity Test.

In an embodiment, if the neat or dilution extract kills more than 30% of the healthy cells (the neat or dilution concentration is higher than IC₃₀), the particles can be altered to reduce porosity and this can be repeated until the particle passes the Extractable Cytotoxicity Test.

A feedback loop can also be based on the Efficacy Determination Protocol (FIG. 1). Details of the Efficacy Determination Protocol are described in Example 6. In some instances, if the degradation of the colorant is less than 90% and the degradation of the material is less than 90%, then the particle is considered passing the Efficacy Determination Protocol.

In some embodiments, the barrier property of the shell can be tuned by choosing the proper shell matrix materials guided by the feedback loop as set forth in FIG. 1. In some embodiments, the protective shell layer comprises a cross-linked polymer. In some embodiments, the cross-linked polymer comprises an organo-modified inorganic polymer. In some embodiments, the organo-modified inorganic polymer comprises a sol-gel organo-modified silicon polymer formed by the condensation of an organo-silanetriol (silicate polymer derived from vinyl trimethoxysilane, organo-silicate). In some embodiments, the organo-silanetriol is vinyl silanetriol resulted from the hydrolysis of a 25% VTMS HCl solution composition.

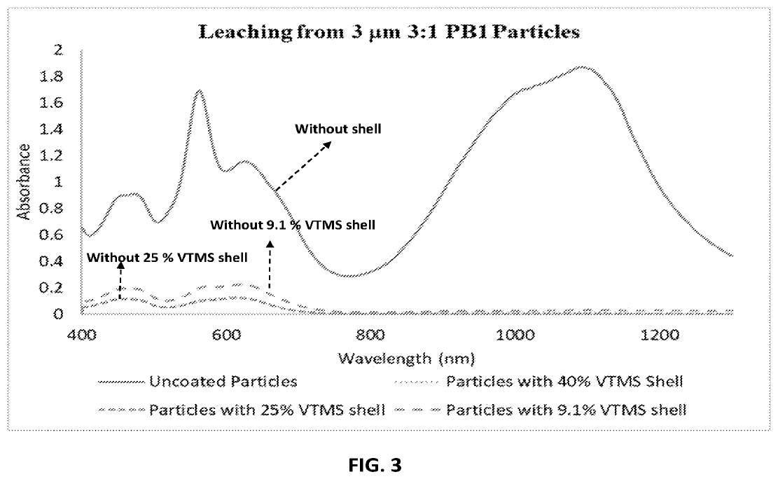

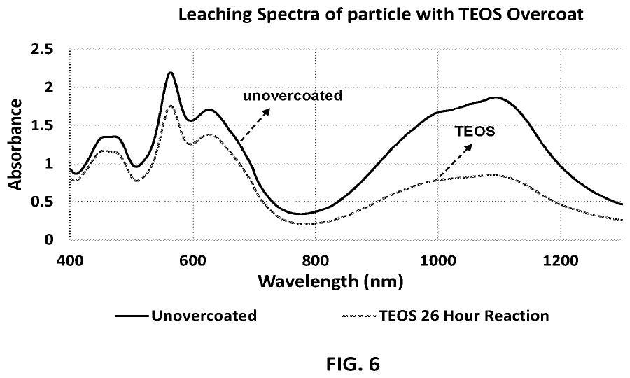

It should be noted for a given particle comprising a carrier, a colorant and a material that interacts with an exogenous source, it is not a given that any cross-linkable polymer will create a shell that would provide the required barrier. A shell made from 25% TEOS solution (TEOS=tetraethylorthosilicate, conventional TEOS derived sol-gel) under the conventional Stober reaction condition did not significantly reduce the concentration of extracted colorant s when subjected to a surfactant-based extractable test (See FIG. 6, Table 8). On the other hand, under the Stober reaction conditions, a shell made from 25% vinyltrimethoxysilane (VTMS) solution when VTMS applied at a 25 wt. % by the weight of the core shell particle provides good retention of colorant s as shown by the significant reduction in the concentration of extracted colorant s when subjected to a surfactant-based extractable test (See FIG. 3, Table 6B).

In some embodiments, the barrier property of the shell can be tuned by selecting organo-silanetriols (e.g., alkylsilanetriols prepared by hydrolyzing the alkyltrimethoxysilane reagent) with different organic groups. In some embodiments, the shell results from the use of an alkyltrimethoxysilane reagent (CnTMS, n is an integer ranging from 1 to 12) in the Stober synthesis. In some embodiments, the shell results from the use of C1-C7 alkyl trimethoxysilane reagent in the Stober synthesis. In some embodiments, the shell results from the use of C1-C7 alkenyl trimethoxysilane reagent in the Stober synthesis. In some embodiments, the shell results from the use of C1-C7 alkynyl trimethoxysilane reagent in the Stober synthesis. In some embodiments, the C1-C7 alkyl group, the C1-C7 alkenyl group, or the C1-C7 alkynyl group may be linear or branched. In some embodiments, the shell results from the use of C2-C6 linear alkyl trimethoxysilane reagent in the Stober synthesis. In some embodiments, the shell results from the use of C2-C4 linear alkyl trimethoxysilane reagent in the Stober synthesis. In some embodiments, the shell results from the use of ethyl (C2) trimethoxysilane reagent in Stober synthesis. In some embodiments, the shell results from the use of vinyltrimethoxysilane reagent in Stober synthesis. In some embodiments, the shell results from the condensation reaction of hydroxymethylsilanetriol prepared by the hydrolysis of hydroxymethyltrichlorosilane.

In some embodiments, the particle shell has tunable porosity by tuning the degree of cross-linking by adjusting the pH value of the reaction medium for the condensation reaction of organo-silanetriol.

In some embodiments, the particle shell has a tunable barrier property by adjusting the shell layer thickness. To tune the level of leakage of payloads from the interior of the particle, the thickness of the sol-gel vinyl modified silicone polymer shell made from VTMS reagent in the Stober reaction was varied by varying the weight ratio of the VTMS reagent to the core-shell particle at 0.083:1, 0.33:1, or 0.66:1 (the amount of VTMS applied is about 7.5 wt. %, about 25 wt. %, or about 40 wt. % by the total weight of the VTMS reagent and the uncoated particle). The same Stober protocol described in Example 1(ii-a) below was used to fabricate the particles having varied shell thickness. The shell comprises sol gel vinyl-modified silicone polymer formed by the condensation reaction of vinylsilanetriol (hydrolysis product of VTMS) under the Stober reaction condition.

In some embodiments, the amount of VTMS applied to form the shell is at a weight percentage by the total weight of the VTMS reagent and the uncoated particle ranging from about 5 wt. % to about 40 wt. %. In some embodiments, the amount of VTMS applied to form the shell is at a weight percentage by the total weight of the VTMS reagent and the uncoated particle ranging from about 6 wt. % to about 40 wt. %. In some embodiments, the amount of VTMS applied to form the shell is at a weight percentage by the total weight of the VTMS reagent and the uncoated particle ranging from about 7 wt. % to about 40 wt. %. In some embodiments, the amount of VTMS applied to form the shell is at a weight percentage by the total weight of the VTMS reagent and the uncoated particle ranging from about 8 wt. % to about 40 wt. %. In some embodiments, the amount of VTMS applied to form the shell is at a weight percentage by the total weight of the VTMS reagent and the uncoated particle ranging from about 9 wt. % to about 40 wt. %. In some embodiments, the amount of VTMS applied to form the shell is at a weight percentage by the total weight of the VTMS reagent and the uncoated particle ranging from about 10 wt. % to about 40 wt. %. In some embodiments, the amount of VTMS applied to form the shell is at a weight percentage by the total weight of the VTMS reagent and the uncoated particle ranging from about 15 wt. % to about 40 wt. %. In some embodiments, the amount of VTMS applied to form the shell is at a weight percentage by the total weight of the VTMS reagent and the uncoated particle ranging from about 25 wt. % to about 40 wt. %. In some embodiments, the amount of VTMS applied to form the shell is at a weight percentage by the total weight of the VTMS reagent and the uncoated particle ranging from about 30 wt. % to about 40 wt. %. In some embodiments, the amount of VTMS applied to form the shell is at a weight percentage by the total weight of the VTMS reagent and the uncoated particle ranging from about 35 wt. % to about 40 wt. %. In some embodiments, the amount of VTMS applied to form the shell is at a weight percentage by the total weight of the VTMS reagent and the uncoated particle ranging from about 12.5 wt. % to about 40 wt. %. In some embodiments, the amount of VTMS applied to form the shell is at a weight percentage by the total weight of the VTMS reagent and the uncoated particle ranging from about 15 wt. % to about 40 wt. %. In some embodiments, the amount of VTMS applied to form the shell is at a weight percentage by the total weight of the VTMS reagent and the uncoated particle ranging from about 17.5 wt. % to about 40 wt. %. In some embodiments, the amount of VTMS applied to form the shell is at a weight percentage by the total weight of the VTMS reagent and the uncoated particle ranging from about 20 wt. % to about 40 wt. %. In some embodiments, the amount of VTMS applied to form the shell is at a weight percentage by the total weight of the VTMS reagent and the uncoated particle ranging from about 22.5 wt. % to about 40 wt. %.In some embodiments, the amount of VTMS applied to form the shell is at a weight percentage by the total weight of the VTMS reagent and the uncoated particle ranging from about 25 wt. % to about 40 wt. %. In some embodiments, the amount of VTMS applied to form the shell is at a weight percentage by the total weight of the VTMS reagent and the uncoated particle ranging from about 27.5 wt. % to about 40 wt. %. In some embodiments, the amount of VTMS applied to form the shell is at a weight percentage by the total weight of the VTMS reagent and the uncoated particle ranging from about 30.0 wt. % to about 40 wt. %. In some embodiments, the amount of VTMS applied to form the shell is at a weight percentage by the total weight of the VTMS reagent and the uncoated particle ranging from about 35 wt. % to about 40 wt. %. In some embodiments, the amount of VTMS applied to form the shell is at a weight percentage by the total weight of the VTMS reagent and the uncoated particle ranging from about 37.5 wt. % to about 40 wt. %.

In some embodiments, the amount of VTMS applied to form the shell is at a weight percentage by the total weight of the VTMS reagent and the uncoated particle ranging from about 5 wt. % to about 35 wt. %. In some embodiments, the amount of VTMS applied to form the shell is at a weight percentage by the total weight of the VTMS reagent and the uncoated particle ranging from about 6 wt. % to about 35 wt. %. In some embodiments, the amount of VTMS applied to form the shell is at a weight percentage by the total weight of the VTMS reagent and the uncoated particle ranging from about 7 wt. % to about 35 wt. %. In some embodiments, the amount of VTMS applied to form the shell is at a weight percentage by the total weight of the VTMS reagent and the uncoated particle ranging from about 8 wt. % to about 35 wt. %. In some embodiments, the amount of VTMS applied to form the shell is at a weight percentage by the total weight of the VTMS reagent and the uncoated particle ranging from about 9 wt. % to about 35 wt. %. In some embodiments, the amount of VTMS applied to form the shell is at a weight percentage by the total weight of the VTMS reagent and the uncoated particle ranging from about 10 wt. % to about 35 wt. %. In some embodiments, the amount of VTMS applied to form the shell is at a weight percentage by the total weight of the VTMS reagent and the uncoated particle ranging from about 15 wt. % to about 35 wt. %. In some embodiments, the amount of VTMS applied to form the shell is at a weight percentage by the total weight of the VTMS reagent and the uncoated particle ranging from about 25 wt. % to about 35 wt. %. In some embodiments, the amount of VTMS applied to form the shell is at a weight percentage by the total weight of the VTMS reagent and the uncoated particle ranging from about 30 wt. % to about 35 wt. %. In some embodiments, the amount of VTMS applied to form the shell is at a weight percentage by the total weight of the VTMS reagent and the uncoated particle ranging from about 12.5 wt. % to about 35 wt. %. In some embodiments, the amount of VTMS applied to form the shell is at a weight percentage by the total weight of the VTMS reagent and the uncoated particle ranging from about 15 wt. % to about 35 wt. %. In some embodiments, the amount of VTMS applied to form the shell is at a weight percentage by the total weight of the VTMS reagent and the uncoated particle ranging from about 17.5 wt. % to about 35 wt. %. In some embodiments, the amount of VTMS applied to form the shell is at a weight percentage by the total weight of the VTMS reagent and the uncoated particle ranging from about 20 wt. % to about 35 wt. %. In some embodiments, the amount of VTMS applied to form the shell is at a weight percentage by the total weight of the VTMS reagent and the uncoated particle ranging from about 22.5 wt. % to about 35 wt. %. In some embodiments, the amount of VTMS applied to form the shell is at a weight percentage by the total weight of the VTMS reagent and the uncoated particle ranging from about 25 wt. % to about 35 wt. %. In some embodiments, the amount of VTMS applied to form the shell is at a weight percentage by the total weight of the VTMS reagent and the uncoated particle ranging from about 27.5 wt. % to about 35 wt. %. In some embodiments, the amount of VTMS applied to form the shell is at a weight percentage by the total weight of the VTMS reagent and the uncoated particle ranging from about 30.0 wt. % to about 35 wt. %.

In some embodiments, the amount of VTMS applied to form the shell is at a weight percentage by the total weight of the VTMS reagent and the uncoated particle ranging from about 5 wt. % to about 30 wt. %. In some embodiments, the amount of VTMS applied to form the shell is at a weight percentage by the total weight of the VTMS reagent and the uncoated particle ranging from about 6 wt. % to about 30 wt. %. In some embodiments, the amount of VTMS applied to form the shell is at a weight percentage by the total weight of the VTMS reagent and the uncoated particle ranging from about 7 wt. % to about 30 wt. %. In some embodiments, the amount of VTMS applied to form the shell is at a weight percentage by the total weight of the VTMS reagent and the uncoated particle ranging from about 8 wt. % to about 30 wt. %. In some embodiments, the amount of VTMS applied to form the shell is at a weight percentage by the total weight of the VTMS reagent and the uncoated particle ranging from about 9 wt. % to about 30 wt. %. In some embodiments, the amount of VTMS applied to form the shell is at a weight percentage by the total weight of the VTMS reagent and the uncoated particle ranging from about 10 wt. % to about 30 wt. %. In some embodiments, the amount of VTMS applied to form the shell is at a weight percentage by the total weight of the VTMS reagent and the uncoated particle ranging from about 12.5 wt. % to about 30 wt. %. In some embodiments, the amount of VTMS applied to form the shell is at a weight percentage by the total weight of the VTMS reagent and the uncoated particle ranging from about 15 wt. % to about 30 wt. %. In some embodiments, the amount of VTMS applied to form the shell is at a weight percentage by the total weight of the VTMS reagent and the uncoated particle ranging from about 17.5 wt. % to about 30 wt. %. In some embodiments, the amount of VTMS applied to form the shell is at a weight percentage by the total weight of the VTMS reagent and the uncoated particle ranging from about 20 wt. % to about 30 wt. %. In some embodiments, the amount of VTMS applied to form the shell is at a weight percentage by the total weight of the VTMS reagent and the uncoated particle ranging from about 22.5 wt. % to about 30 wt. %. In some embodiments, the amount of VTMS applied to form the shell is at a weight percentage by the total weight of the VTMS reagent and the uncoated particle ranging from about 25 wt. % to about 30 wt. %. In some embodiments, the amount of VTMS applied to form the shell is at a weight percentage by the total weight of the VTMS reagent and the uncoated particle ranging from about 27.5 wt. % to about 30 wt. %.

In some embodiments, the amount of VTMS applied to form the shell is at a weight percentage by the total weight of the VTMS reagent and the uncoated particle ranging from about 21.0 wt. % to about 29.0 wt. %. In some embodiments, the amount of VTMS applied to form the shell is at a weight percentage by the total weight of the VTMS reagent and the uncoated particle ranging from about 22.0 wt. % to about 26.0 wt. %. In some embodiments, the amount of VTMS applied to form the shell is at a weight percentage by the total weight of the VTMS reagent and the uncoated particle ranging from about 23.0 wt. % to about 26.0 wt. %. In some embodiments, the amount of VTMS applied to form the shell is at a weight percentage by the total weight of the VTMS reagent and the uncoated particle ranging from about 24.0 wt. % to about 26.0 wt. %.

In some embodiments, the amount of VTMS applied to form the shell is at a weight percentage by the total weight of the VTMS reagent and the uncoated particle ranging from about 5 wt. % to about 25 wt. %. In some embodiments, the amount of VTMS applied to form the shell is at a weight percentage by the total weight of the VTMS reagent and the uncoated particle ranging from about 7.5 wt. % to about 25 wt. %. In some embodiments, the amount of VTMS applied to form the shell is at a weight percentage by the total weight of the VTMS reagent and the uncoated particle ranging from about 6 wt. % to about 25 wt. %. In some embodiments, the amount of VTMS applied to form the shell is at a weight percentage by the total weight of the VTMS reagent and the uncoated particle ranging from about 7 wt. % to about 25 wt. %. In some embodiments, the amount of VTMS applied to form the shell is at a weight percentage by the total weight of the VTMS reagent and the uncoated particle ranging from about 8 wt. % to about 25 wt. %. In some embodiments, the amount of VTMS applied to form the shell is at a weight percentage by the total weight of the VTMS reagent and the uncoated particle ranging from about 9 wt. % to about 25 wt. %. In some embodiments, the amount of VTMS applied to form the shell is at a weight percentage by the total weight of the VTMS reagent and the uncoated particle ranging from about 10 wt. % to about 25 wt. %. In some embodiments, the amount of VTMS applied to form the shell is at a weight percentage by the total weight of the VTMS reagent and the uncoated particle ranging from about 15 wt. % to about 25 wt. %. In some embodiments, the amount of VTMS applied to form the shell is at a weight percentage by the total weight of the VTMS reagent and the uncoated particle ranging from about 12.5 wt. % to about 25 wt. %. In some embodiments, the amount of VTMS applied to form the shell is at a weight percentage by the total weight of the VTMS reagent and the uncoated particle ranging from about 15 wt. % to about 25 wt. %. In some embodiments, the amount of VTMS applied to form the shell is at a weight percentage by the total weight of the VTMS reagent and the uncoated particle ranging from about 17.5 wt. % to about 25 wt. %. In some embodiments, the amount of VTMS applied to form the shell is at a weight percentage by the total weight of the VTMS reagent and the uncoated particle ranging from about 20 wt. % to about 25 wt. %.

In some embodiments, the amount of VTMS applied to form the shell is at a weight percentage by the total weight of the VTMS reagent and the uncoated particle selected from the group of about 5.0 wt. %, about 5.5 wt. %, about 6.0 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, about 25.0 wt. %, about 25.5 wt. %, about 26.0 wt. %, about 26.5 wt. %, about 27.0 wt. %, about 27.5 wt. %, about 28.0 wt. %, about 28.5 wt. %, about 29.0 wt. %, about 29.5 wt. %, about 30.0 wt. %, about 30.5 wt. %, about 31.0 wt. %, about 31.5 wt. %, about 32.0 wt. %, about 32.5 wt. %, about 33.0 wt. %, about 33.5 wt. %, about 34.0 wt. %, about 34.5 wt. %, about 35.0 wt. %, about 35.5 wt. %, about 36.0 wt. %, about 36.5 wt. %, about 37.0 wt. %, about 37.5 wt. %, about 38.0 wt. %, about 38.5 wt. %, about 39.0 wt. %, about 39.5 wt. %, or 40.0 wt. %. In an embodiment, the amount of VTMS applied to form the shell is about 7.5 wt. % by the total weight of the VTMS reagent and the uncoated particle. In an embodiment, the amount of VTMS applied to form the shell is about 10.0 wt. % by the total weight of the VTMS reagent and the uncoated particle. In an embodiment, the amount of VTMS applied to form the shell is about 15.0 wt. % by the total weight of the VTMS reagent and the uncoated particle. In an embodiment, the amount of VTMS applied to form the shell is about 20.0 wt. % by the total weight of the VTMS reagent and the uncoated particle. In an embodiment, the amount of VTMS applied to form the shell is about 25.0 wt. % by the total weight of the VTMS reagent and the uncoated particle. In an embodiment, the amount of VTMS applied to form the shell is about 30.0 wt. % by the total weight of the VTMS reagent and the uncoated particle.

In some embodiments, the amount of VTMS applied to form the shell is about 8.3 wt. % by the total weight of the uncoated particle (weight ratio VTMS/uncoated particle =0.083:1). In some embodiments, the amount of VTMS applied to form the shell is about 33.0 wt. % by the total weight of the uncoated particle (weight ratio VTMS/uncoated particle =0.33:1). In some embodiments, the amount of VTMS applied to form the shell is about 66.0 wt. % by the total weight of the uncoated particle (weight ratio VTMS/uncoated particle =0.66:1). In some embodiments, the amount of VTMS applied to form the shell ranges from about 8.3 wt. % to about 66 wt. % by the total weight of the uncoated particle (weight ratio VTMS/uncoated particle ranges from 0.083:1 to 0.66:1).

The results in Tables 6A, 6B, and 6C below showed the increase of the shell thickness would reduce the level of leaching of the payloads, for example, a particle having a 25% VTMS shell exhibited better results in reducing the leakage of dye as compared with a particle having a 9.1% VTMS shell (see Tables 6B and 6C below). However, the further increase the amount of VTMS from about 25 wt. % to about 40 wt. % by the total weight of the VTMS reagent and the uncoated particle did not yield improved shell performance on reducing dye leaching as compared to the particle having a 25 VTMS shell (Tables 6A and 6B).

In some embodiments, the shell layer is present in an amount of greater than 10.0 wt. % of the total weight of the uncoated particles. In some embodiments, the shell layer is present in an amount of greater than 20.0 wt. % of the total weight of the uncoated particles. In some embodiments, the shell layer is present in an amount of greater than 30.0 wt. % of the total weight of the uncoated particles. In some embodiments, the shell layer is present in an amount of greater than 40.0 wt. % of the total weight of the uncoated particles. In some embodiments, the shell layer is present in an amount of greater than 50.0 wt. % of the total weight of the uncoated particles. In some embodiments, the shell layer is present in an amount of greater than 60.0 wt. % of the total weight of the uncoated particles. In some embodiments, the shell layer is present in an amount in a range from about 10.0 wt. % to about 200 wt. % of the total weight of the uncoated particles. In some embodiments, the shell layer is present in an amount ranging from about 20.0 wt. % to about 100 wt. % of the total weight of the uncoated particles. In some embodiments, the shell layer is present in an amount ranging from about 20.0 wt. % to about 120 wt. % of the total weight of the uncoated particles. In some embodiments, the shell layer is present in an amount ranging from about 20.0 wt. % to about 130 wt. % of the total weight of the uncoated particles. In some embodiments, the shell layer is present in an amount ranging from about 20.0 wt. % to about 140 wt. % of the total weight of the uncoated particles. In some embodiments, the shell layer is present in an amount ranging from about 20.0 wt. % to about 150 wt. % of the total weight of the uncoated particles. In some embodiments, the shell layer is present in an amount ranging from about 20.0 wt. % to about 200 wt. % of the total weight of the uncoated particles. In some embodiments, the shell layer is present in an amount ranging from about 30.0 wt. % to about 100 wt. % of the total weight of the uncoated particles. In some embodiments, the shell layer is present in an amount ranging from about 40.0 wt. % to about 100 wt. % of the total weight of the uncoated particles. In some embodiments, the shell layer is present in an amount ranging from about 60.0 wt. % to about 100 wt. % of the total weight of the uncoated particles. In some embodiments, the shell layer is present in an amount ranging from about 70.0 wt. % to about 100 wt. % of the total weight of the uncoated particles.

In some embodiments, the amount of shell is at a weight percentage by the total weight of the shell and the uncoated particle ranging from about 5 wt. % to about 40 wt. %. In some embodiments, the amount of shell is at a weight percentage by the total weight of the shell and the uncoated particle ranging from about 6 wt. % to about 40 wt. %. In some embodiments, the amount of shell is at a weight percentage by the total weight of the shell and the uncoated particle ranging from about 7 wt. % to about 40 wt. %. In some embodiments, the amount of shell is at a weight percentage by the total weight of the shell and the uncoated particle ranging from about 8 wt. % to about 40 wt. %. In some embodiments, the amount of shell is at a weight percentage by the total weight of the shell and the uncoated particle ranging from about 9 wt. % to about 40 wt. %. In some embodiments, the amount of shell is at a weight percentage by the total weight of the shell and the uncoated particle ranging from about 10 wt. % to about 40 wt. %. In some embodiments, the amount of shell is at a weight percentage by the total weight of the shell and the uncoated particle ranging from about 15 wt. % to about 40 wt. %. In some embodiments, the amount of shell is at a weight percentage by the total weight of the shell and the uncoated particle ranging from about 25 wt. % to about 40 wt. %. In some embodiments, the amount of shell is at a weight percentage by the total weight of the shell and the uncoated particle ranging from about 30 wt. % to about 40 wt. %. In some embodiments, the amount of shell is at a weight percentage by the total weight of the shell and the uncoated particle ranging from about 35 wt. % to about 40 wt. %. In some embodiments, the amount of shell is at a weight percentage by the total weight of the shell and the uncoated particle ranging from about 12.5 wt. % to about 40 wt. %. In some embodiments, the amount of shell is at a weight percentage by the total weight of the shell and the uncoated particle ranging from about 15 wt. % to about 40 wt. %. In some embodiments, the amount of shell is at a weight percentage by the total weight of the shell and the uncoated particle ranging from about 17.5 wt. % to about 40 wt. %. In some embodiments, the amount of shell is at a weight percentage by the total weight of the shell and the uncoated particle ranging from about 20 wt. % to about 40 wt. %. In some embodiments, the amount of shell is at a weight percentage by the total weight of the shell and the uncoated particle ranging from about 22.5 wt. % to about 40 wt. %.In some embodiments, the amount of shell is at a weight percentage by the total weight of the shell and the uncoated particle ranging from about 25 wt. % to about 40 wt. %. In some embodiments, the amount of shell is at a weight percentage by the total weight of the shell and the uncoated particle ranging from about 27.5 wt. % to about 40 wt. %. In some embodiments, the amount of shell is at a weight percentage by the total weight of the shell and the uncoated particle ranging from about 30.0 wt. % to about 40 wt. %. In some embodiments, the amount of shell is at a weight percentage by the total weight of the shell and the uncoated particle ranging from about 35 wt. % to about 40 wt. %. In some embodiments, the amount of shell is at a weight percentage by the total weight of the shell and the uncoated particle ranging from about 37.5 wt. % to about 40 wt. %.

In some embodiments, the amount of shell is at a weight percentage by the total weight of the shell and the uncoated particle ranging from about 5 wt. % to about 35 wt. %. In some embodiments, the amount of shell is at a weight percentage by the total weight of the shell and the uncoated particle ranging from about 6 wt. % to about 35 wt. %. In some embodiments, the amount of shell is at a weight percentage by the total weight of the shell and the uncoated particle ranging from about 7 wt. % to about 35 wt. %. In some embodiments, the amount of shell is at a weight percentage by the total weight of the shell and the uncoated particle ranging from about 8 wt. % to about 35 wt. %. In some embodiments, the amount of shell is at a weight percentage by the total weight of the shell and the uncoated particle ranging from about 9 wt. % to about 35 wt. %. In some embodiments, the amount of shell is at a weight percentage by the total weight of the shell and the uncoated particle ranging from about 10 wt. % to about 35 wt. %. In some embodiments, the amount of shell is at a weight percentage by the total weight of the shell and the uncoated particle ranging from about 15 wt. % to about 35 wt. %. In some embodiments, the amount of shell is at a weight percentage by the total weight of the shell and the uncoated particle ranging from about 25 wt. % to about 35 wt. %. In some embodiments, the amount of shell is at a weight percentage by the total weight of the shell and the uncoated particle ranging from about 30 wt. % to about 35 wt. %. In some embodiments, the amount of shell is at a weight percentage by the total weight of the shell and the uncoated particle ranging from about 12.5 wt. % to about 35 wt. %. In some embodiments, the amount of shell is at a weight percentage by the total weight of the shell and the uncoated particle ranging from about 15 wt. % to about 35 wt. %. In some embodiments, the amount of shell is at a weight percentage by the total weight of the shell and the uncoated particle ranging from about 17.5 wt. % to about 35 wt. %. In some embodiments, the amount of shell is at a weight percentage by the total weight of the shell and the uncoated particle ranging from about 20 wt. % to about 35 wt. %. In some embodiments, the amount of shell is at a weight percentage by the total weight of the shell and the uncoated particle ranging from about 22.5 wt. % to about 35 wt. %. In some embodiments, the amount of shell is at a weight percentage by the total weight of the shell and the uncoated particle ranging from about 25 wt. % to about 35 wt. %. In some embodiments, the amount of shell is at a weight percentage by the total weight of the shell and the uncoated particle ranging from about 27.5 wt. % to about 35 wt. %. In some embodiments, the amount of shell is at a weight percentage by the total weight of the shell and the uncoated particle ranging from about 30.0 wt. % to about 35 wt. %.

In some embodiments, the amount of shell is at a weight percentage by the total weight of the shell and the uncoated particle ranging from about 5 wt. % to about 30 wt. %. In some embodiments, the amount of shell is at a weight percentage by the total weight of the shell and the uncoated particle ranging from about 6 wt. % to about 30 wt. %. In some embodiments, the amount of shell is at a weight percentage by the total weight of the shell and the uncoated particle ranging from about 7 wt. % to about 30 wt. %. In some embodiments, the amount of shell is at a weight percentage by the total weight of the shell and the uncoated particle ranging from about 8 wt. % to about 30 wt. %. In some embodiments, the amount of shell is at a weight percentage by the total weight of the shell and the uncoated particle ranging from about 9 wt. % to about 30 wt. %. In some embodiments, the amount of shell is at a weight percentage by the total weight of the shell and the uncoated particle ranging from about 10 wt. % to about 30 wt. %. In some embodiments, the amount of shell is at a weight percentage by the total weight of the shell and the uncoated particle ranging from about 12.5 wt. % to about 30 wt. %. In some embodiments, the amount of shell is at a weight percentage by the total weight of the shell and the uncoated particle ranging from about 15 wt. % to about 30 wt. %. In some embodiments, the amount of shell is at a weight percentage by the total weight of the shell and the uncoated particle ranging from about 17.5 wt. % to about 30 wt. %. In some embodiments, the amount of shell is at a weight percentage by the total weight of the shell and the uncoated particle ranging from about 20 wt. % to about 30 wt. %. In some embodiments, the amount of shell is at a weight percentage by the total weight of the shell and the uncoated particle ranging from about 22.5 wt. % to about 30 wt. %. In some embodiments, the amount of shell is at a weight percentage by the total weight of the shell and the uncoated particle ranging from about 25 wt. % to about 30 wt. %. In some embodiments, the amount of shell is at a weight percentage by the total weight of the shell and the uncoated particle ranging from about 27.5 wt. % to about 30 wt. %.

In some embodiments, the amount of shell is at a weight percentage by the total weight of the shell and the uncoated particle ranging from about 21.0 wt. % to about 29.0 wt. %. In some embodiments, the amount of shell is at a weight percentage by the total weight of the shell and the uncoated particle ranging from about 22.0 wt. % to about 26.0 wt. %. In some embodiments, the amount of shell is at a weight percentage by the total weight of the shell and the uncoated particle ranging from about 23.0 wt. % to about 26.0 wt. %. In some embodiments, the amount of shell is at a weight percentage by the total weight of the shell and the uncoated particle ranging from about 24.0 wt. % to about 26.0 wt. %.

In some embodiments, the amount of shell is at a weight percentage by the total weight of the shell and the uncoated particle ranging from about 5 wt. % to about 25 wt. %. In some embodiments, the amount of shell is at a weight percentage by the total weight of the shell and the uncoated particle ranging from about 7.5 wt. % to about 25 wt. %. In some embodiments, the amount of shell is at a weight percentage by the total weight of the shell and the uncoated particle ranging from about 6 wt. % to about 25 wt. %. In some embodiments, the amount of shell is at a weight percentage by the total weight of the shell and the uncoated particle ranging from about 7 wt. % to about 25 wt. %. In some embodiments, the amount of shell is at a weight percentage by the total weight of the shell and the uncoated particle ranging from about 8 wt. % to about 25 wt. %. In some embodiments, the amount of shell is at a weight percentage by the total weight of the shell and the uncoated particle ranging from about 9 wt. % to about 25 wt. %. In some embodiments, the amount of shell is at a weight percentage by the total weight of the shell and the uncoated particle ranging from about 10 wt. % to about 25 wt. %. In some embodiments, the amount of shell is at a weight percentage by the total weight of the shell and the uncoated particle ranging from about 15 wt. % to about 25 wt. %. In some embodiments, the amount of shell is at a weight percentage by the total weight of the shell and the uncoated particle ranging from about 12.5 wt. % to about 25 wt. %. In some embodiments, the amount of shell is at a weight percentage by the total weight of the shell and the uncoated particle ranging from about 15 wt. % to about 25 wt. %. In some embodiments, the amount of shell is at a weight percentage by the total weight of the shell and the uncoated particle ranging from about 17.5 wt. % to about 25 wt. %. In some embodiments, the amount of shell is at a weight percentage by the total weight of the shell and the uncoated particle ranging from about 20 wt. % to about 25 wt. %.

In some embodiments, the amount of shell is at a weight percentage by the total weight of the shell and the uncoated particle selected from the group of about 5.0 wt. %, about 5.5 wt. %, about 6.0 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, about 25.0 wt. %, about 25.5 wt. %, about 26.0 wt. %, about 26.5 wt. %, about 27.0 wt. %, about 27.5 wt. %, about 28.0 wt. %, about 28.5 wt. %, about 29.0 wt. %, about 29.5 wt. %, about 30.0 wt. %, about 30.5 wt. %, about 31.0 wt. %, about 31.5 wt. %, about 32.0 wt. %, about 32.5 wt. %, about 33.0 wt. %, about 33.5 wt. %, about 34.0 wt. %, about 34.5 wt. %, about 35.0 wt. %, about 35.5 wt. %, about 36.0 wt. %, about 36.5 wt. %, about 37.0 wt. %, about 37.5 wt. %, about 38.0 wt. %, about 38.5 wt. %, about 39.0 wt. %, about 39.5 wt. %, or 40.0 wt. %. In an embodiment, the amount of shell is about 7.5 wt. % by the total weight of the shell and the uncoated particle. In an embodiment, the amount of shell is about 10.0 wt. % by the total weight of the shell and the uncoated particle. In an embodiment, the amount of shell 1 is about 15.0 wt. % by the total weight of the shell and the uncoated particle. In an embodiment, the amount of shell is about 20.0 wt. % by the total weight of the shell and the uncoated particle. In an embodiment, the amount of shell is about 25.0 wt. % by the total weight of the shell and the uncoated particle. In an embodiment, the amount of shell is about 30.0 wt. % by the total weight of the shell and the uncoated particle.

In some embodiments, the shell layer is present in an amount (e.g., 10 wt. % of the total weight of the uncoated particles) that forms an imperfect shell that is unable to completely prevent leakage of components or that meets the cytotoxicity IC₃₀ criteria as set forth above. In some embodiments, the shell layer is present in an amount of about 100 wt. % of the total weight of the uncoated particles. In some embodiments, the shell layer is present in an amount of about 200 wt. % of the total weight of the uncoated particles. In some embodiments, the shell layer is present in an amount in selected from the group of about 10.0 wt. %, about 15.0 wt. %, about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, about 100 wt. %, about 110 wt. %, about 115 wt. %, about 120 wt. %, about 125 wt. %, about 130 wt. %, about 135 wt. %, about 140 wt. %, about 145 wt. %, about 150 wt. %, about 155 wt. %, about 160 wt. %, about 165 wt. %, about 170 wt. %, about 175 wt. %, about 180 wt. %, about 185 wt. %, about 190 wt. %, about 195 wt. %, about 200 wt. % of the total weight of the uncoated particles. In some embodiments, the shell layer is present in an amount in a range from 10.0 wt. % to about 35.0 wt. % of the total weight of the uncoated particles. In some embodiments, the shell is present in an amount of about 35.0 wt. % of the total weight of the uncoated particles.

It should be noted that particle cytotoxicity and efficacy are determined respectively by the Extractable Cytotoxicity Test and the Efficacy Determination Protocol. To reduce the number of each of these tests, it is advantageous to establish a surfactant-based extractable test to estimate the leached concentration outside the particle and to initially evaluate the effects of particle structure variation by measuring the reduction in the leached concentration before performing the Extractable Cytotoxicity Test and the Efficacy Determination Protocol.

In some embodiments, the particle has a substantially low leakage of colorant such that the particle has low cytotoxicity. In some embodiments, the substantial low leakage of colorant refers to a colorant leakage being less than about 20.0%. In some embodiments, the leakage of colorant is less than about 15.0%. In some embodiments, the leakage of colorant is less than about 10.0%. In some embodiments, the leakage of colorant is less than about 5.0%. In some embodiments, the leakage of colorant is less than about 4.0%. In some embodiments, the leakage of colorant is less than about 3.0%. In some embodiments, the leakage of colorant is less than about 2.0%. In some embodiments, the leakage of colorant is less than about 1.0%. In some embodiments, the leakage of colorant is less than about 0.1%. In some embodiments, the leakage of colorant is less than about 0.01%. In some embodiments, the leakage of the colorant is 0%. In some embodiments, the leakage of the colorant is less than a percentage value selected from the group of: about 0.01%, 0.1%, 0.5%, 1.0%, 1.5%, 2.0%, 2.5%, 3.0 %, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, or 25.0%. In some embodiments, the leakage of the colorant ranging from about 0.01% to about 5.0%. In some embodiments, the leakage of the colorant ranging from about 0.01% to about 4.0%. In some embodiments, the leakage of the colorant ranging from about 0.01% to about 3.0%. In some embodiments, the leakage of the colorant ranging from about 0.01% to about 2.0%. In some embodiments, the leakage of the colorant ranging from about 0.01% to about 1.0%. In some embodiments, the leakage of the colorant ranging from about 0.01% to about 0.1%. In some embodiments, the leakage of the colorant ranging from about 0.1% to about 5.0%. In some embodiments, the leakage of the colorant ranging from about 0.1% to about 4.0%. In some embodiments, the leakage of the colorant ranging from about 0.1% to about 3.0%. In some embodiments, the leakage of the colorant ranging from about 0.1% to about 2.0%. In some embodiments, the leakage of the colorant ranging from about 0.1% to about 1.0%.

In some embodiments, the level of the colorant and/or the material (e.g., the payload) leaching from particles with or without shell can be tuned by adjusting the weight ratio of the carrier to the colorant and/or the material. In some embodiments, the level of the colorant and/or the material leakage can be reduced by increasing the weight ratio of the carrier to the colorant and/or the material. In some embodiments, the cytotoxicity of the particle is reduced due to the reduced level of the leakage of the colorant and/or the material as a result of increased weight ratio of the carrier to the payloads. In some embodiments, the weight ratio of polymer carrier to the dye also has an effect on the cytotoxicity caused by the leached dye from the polymer particle due to the inherent porosity and free volume of the polymeric particle matrix.

In some embodiments, the particle comprises the carrier to the payload (e.g., dye) in a weight ratio ranging from 1:10 to 10:1. In some embodiments, the weight ratio of the carrier to the payload ranges from 1:1 to 7:1. In some embodiments, the weight ratio of the carrier to the payload ranges from 2:1 to 7:1. In some embodiments, the weight ratio of the carrier to the payload ranges from 3:1 to 7:1. In some embodiments, the weight ratio of the carrier to the payload ranges from 4:1 to 7:1. In some embodiments, the weight ratio of the carrier to the payload ranges from 5:1 to 7:1. In some embodiments, the weight ratio of the carrier to the payload ranges from 6:1 to 7:1. In some embodiments, the weight ratio of the carrier to the payload ranges from 1:7 to 7:1. In some embodiments, the weight ratio of the carrier to the payload ranges from 1:5 to 5:1. In some embodiments, the weight ratio of the carrier to the payload ranges from 1:3 to 3:1. In some embodiments, the weight ratio of the carrier to the payload is a range selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1. In some embodiments, the weight ratio of the carrier to the payload is a range selected from the group of 1:1, 2:1, 3:1, 5:1, or 7:1. In some embodiments, the weight ratio of the carrier to the payload is 2:1. In some embodiments, the weight ratio of the carrier to the payload is 3:1. In some embodiments, the weight ratio of the carrier to the payload is 5:1. In some embodiments, the weight ratio of the carrier to the payload is 7:1.

In some embodiments, the particle exhibits stability and carrier matrix integrity such that the degradation of the colorant and/or the material ranges from about 5.0% to about 95% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is 0% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 90% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 85% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 80% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 75% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 70% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 65% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 60% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 55% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 50% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 45% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 40% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 30% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 20% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 10% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 5% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 1% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material is less than 0.1% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material ranges from about 0.01% to 10.0% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material ranges from about 0.01% to 5.0% as measured by the Efficacy Determination Protocol after incubating the particles in the extraction medium (serum) for 24 hours at 37° C. In some embodiments, the particle exhibits stability and carrier matrix integrity such that the degradation of the colorant and/or the material ranges from about 0.01% to 1.0% as measured by the Efficacy Determination Protocol after incubating the particles in the extraction medium (serum) for 24 hours at 37° C. In some embodiments, the particle exhibits stability such that the colorant and the material respectively has a degree of degradation selected from the group of about 0%, about 0.01%, about 0.1%, about 0.5%, about 1.0%, about 2.0%, about 3.0%, about 5.0%, about 6.0%, about 7.0%, about 8.0%, about 9.0%, about 10.0%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30% , about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53% , about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76% , about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%. In some embodiments, the particle exhibits stability such that the colorant and the material respectively has a degree of degradation selected from the group of about 5.0%, about 10%, about 15%, about 20%, about 25%, about 30%, about 35%, about 40%, about 45%, about 50%, about 55%, about 60%, about 65%, about 70% , about 75%, about 80%, about 85%, about 90%, or about 95%. In some embodiments, the particle exhibits stability such that the degree of the degradation of the colorant and the material respectively ranges from about 25% to about 50%. In some embodiments, the particle exhibits stability such that the degradation of the colorant and the material respectively is less than about 25.0% as measured by the Efficacy Determination Protocol. In some embodiments, the colorant and the material respectively has a degree of degradation in a range selected from the group of: less than about 25.0%, less than about 20.0%, less than about 15.0%, less than about 10.0%, less than about 5.0%, less than about 1.0%, less than about 0.5%, less than about 0.1%, less than about 0.01%, 0% as determined by the Efficacy Determination Protocol. In some embodiments, the colorant and the material respectively has a degree of degradation less than about 10.0% as determined by the Efficacy Determination Protocol. In some embodiments, the colorant and the material respectively has a degree of degradation less than about 5.0%. In some embodiments, the colorant and the material respectively has a degree of degradation less than about 1.0%. In some embodiments, the colorant and the material respectively has a degree of degradation less than about 0.1%.

In one embodiment, this disclosure provides a particle suitable for tissue marking applications (e.g., tattoo ink) comprising (a) a core comprising a carrier, a material, and a visible color dye, (b) a shell enclosing the core, wherein the material absorbs radiation at infrared wavelengths (IR absorbing agent), wherein the visible color dye is transformed to a colorless dye when the material absorbs radiation at infrared wavelengths, wherein the visible color dye and the material exhibits stability such that the colorant and the material in the particle exhibit stability such that the particle is considered passing the Efficacy Determination Protocol; and wherein the particle structure is constructed such that it passes the Extractable Cytotoxicity Test.

In some embodiments, the IR absorbing agent is a tetrakis aminium dye. In some embodiments, the IR absorbing agent is a zinc iron phosphate pigment.

In some embodiments, the tetrakis aminium dye is Epolight™ 1178. In some embodiments, the IR absorbing agent is a tetrakis aminium dye has minimal visible color. In some embodiments, the tetrakis aminium dye is Epolight™ 1117 ((hexafluorophosphate as counterion, molecular weight, 1211 Da, peak absorption 1098 nm).

In some embodiments, the materials are inorganic IR absorbing agents with near-infrared absorbing properties selected from zinc copper phosphate pigment ((Zn, Cu)₂P₂O₇), zinc iron phosphate pigment ((Zn, Fe)₃(PO₄)₂), magnesium copper silicate ((Mg, Cu)₂Si₂O₆ solid solutions), and combinations thereof. In some embodiments, the inorganic IR absorbing agent is a zinc iron phosphate pigment ((Zn, Fe)3(PO4)2).

In some embodiments, the IR absorbing agent is in close proximity to the visible color dye within the carrier matrix. In some embodiments, the IR absorbing agent and the color dye are admixed within the carrier to form a homogeneous dispersion or a solid solution. In some embodiments, the IR absorbing agent and the color dye may have oppositely charged functional group(s) (e.g., IR absorbing agent is positively charged tetrakis aminium dye, and dye of visible color is negatively charged phosphate) such that the two components can be drawn to close proximity by ionic electrostatic interactions.

In some embodiments, the particles comprise IR absorbing agent in an amount ranging from about 5.0 wt. % to about 15.0 wt. % by the total weight of the particles. In some embodiments, the IR absorbing agent is present in an amount ranging from about 5.5 wt. % to about 15.0 wt. % by the total weight of the particle. In some embodiments, the IR absorbing agent is present in an amount ranging from about 6.0 wt. % to about 15.0 wt. % by the total weight of the particle. In some embodiments, the IR absorbing agent is present in an amount ranging from about 6.5 wt. % to about 15.0 wt. % by the total weight of the particle. In some embodiments, the IR absorbing agent is present in an amount ranging from about 7.0 wt. % to about 15.0 wt. % by the total weight of the particle. In some embodiments, the IR absorbing agent is present in an amount ranging from about 7.5 wt. % to about 15.0 wt. % by the total weight of the particle. In some embodiments, the IR absorbing agent is present in an amount ranging from about 8.0 wt. % to about 15.0 wt. % by the total weight of the particle. In some embodiments, the IR absorbing agent is present in an amount ranging from about 8.5 wt. % to about 15.0 wt. % by the total weight of the particle. In some embodiments, the IR absorbing agent is present in an amount ranging from about 9.0 wt. % to about 15.0 wt. % by the total weight of the particle. In some embodiments, the IR absorbing agent is present in an amount ranging from about 9.5 wt. % to about 15.0 wt. % by the total weight of the particle. In some embodiments, the IR absorbing agent is present in an amount ranging from about 10.0 wt. % to about 15.0 wt. % by the total weight of the particle. In some embodiments, the IR absorbing agent is present in an amount ranging from about 10.5 wt. % to about 15.0 wt. % by the total weight of the particle. In some embodiments, the IR absorbing agent is present in an amount ranging from about 11.0 wt. % to about 15.0 wt. % by the total weight of the particle. In some embodiments, the IR absorbing agent is present in an amount ranging from about 11.5 wt. % to about 15.0 wt. % by the total weight of the particle. In some embodiments, the IR absorbing agent is present in an amount ranging from about 12.0 wt. % to about 15.0 wt. % by the total weight of the particle. In some embodiments, the IR absorbing agent is present in an amount ranging from about 12.5 wt. % to about 15.0 wt. % by the total weight of the particle. In some embodiments, the IR absorbing agent is present in an amount ranging from about 13.0 wt. % to about 15.0 wt. % by the total weight of the particle. In some embodiments, the IR absorbing agent is present in an amount ranging from about 13.5 wt. % to about 15.0 wt. % by the total weight of the particle. In some embodiments, the IR absorbing agent is present in an amount ranging from about 14.0 wt. % to about 15.0 wt. % by the total weight of the particle. In some embodiments, the IR absorbing agent is present in an amount ranging from about 5.0 wt. % to about 14.0 wt. % by the total weight of the particle. In some embodiments, the IR absorbing agent is present in an amount ranging from about 5.5 wt. % to about 14.0 wt. % by the total weight of the particle. In some embodiments, the IR absorbing agent is present in an amount ranging from about 6.0 wt. % to about 14.0 wt. % by the total weight of the particle. In some embodiments, the IR absorbing agent is present in an amount ranging from about 6.5 wt. % to about 14.0 wt. % by the total weight of the particle. In some embodiments, the IR absorbing agent is present in an amount ranging from about 7.0 wt. % to about 14.0 wt. % by the total weight of the particle. In some embodiments, the IR absorbing agent is present in an amount ranging from about 7.5 wt. % to about 14.0 wt. % by the total weight of the particle. In some embodiments, the IR absorbing agent is present in an amount ranging from about 8.0 wt. % to about 14.0 wt. % by the total weight of the particle. In some embodiments, the IR absorbing agent is present in an amount ranging from about 8.5 wt. % to about 14.0 wt. % by the total weight of the particle. In some embodiments, the IR absorbing agent is present in an amount ranging from about 9.0 wt. % to about 14.0 wt. % by the total weight of the particle. In some embodiments, the IR absorbing agent is present in an amount ranging from about 9.5 wt. % to about 14.0 wt. % by the total weight of the particle. In some embodiments, the IR absorbing agent is present in an amount ranging from about 10.0 wt. % to about 14.0 wt. % by the total weight of the particle. In some embodiments, the IR absorbing agent is present in an amount ranging from about 10.5 wt. % to about 14.0 wt. % by the total weight of the particle. In some embodiments, the IR absorbing agent is present in an amount ranging from about 11.0 wt. % to about 14.0 wt. % by the total weight of the particle. In some embodiments, the IR absorbing agent is present in an amount ranging from about 11.5 wt. % to about 14.0 wt. % by the total weight of the particle. In some embodiments, the IR absorbing agent is present in an amount ranging from about 12.0 wt. % to about 14.0 wt. % by the total weight of the particle. In some embodiments, the IR absorbing agent is present in an amount ranging from about 12.5 wt. % to about 14.0 wt. % by the total weight of the particle. In some embodiments, the IR absorbing agent is present in an amount ranging from about 13.0 wt. % to about 14.0 wt. % by the total weight of the particle. In some embodiments, the IR absorbing agent is present in an amount ranging from about 13.5 wt. % to about 14.0 wt. % by the total weight of the particle. In some embodiments, the IR absorbing agent is present in an amount ranging from about 5.0 wt. % to about 13.0 wt. % by the total weight of the particle. In some embodiments, the IR absorbing agent is present in an amount ranging from about 5.5 wt. % to about 13.0 wt. % by the total weight of the particle. In some embodiments, the IR absorbing agent is present in an amount ranging from about 6.0 wt. % to about 13.0 wt. % by the total weight of the particle. In some embodiments, the IR absorbing agent is present in an amount ranging from about 6.5 wt. % to about 13.0 wt. % by the total weight of the particle. In some embodiments, the IR absorbing agent is present in an amount ranging from about 7.0 wt. % to about 13.0 wt. % by the total weight of the particle. In some embodiments, the IR absorbing agent is present in an amount ranging from about 7.5 wt. % to about 13.0 wt. % by the total weight of the particle. In some embodiments, the IR absorbing agent is present in an amount ranging from about 8.0 wt. % to about 13.0 wt. % by the total weight of the particle. In some embodiments, the IR absorbing agent is present in an amount ranging from about 8.5 wt. % to about 13.0 wt. % by the total weight of the particle. In some embodiments, the IR absorbing agent is present in an amount ranging from about 9.0 wt. % to about 13.0 wt. % by the total weight of the particle. In some embodiments, the IR absorbing agent is present in an amount ranging from about 9.5 wt. % to about 13.0 wt. % by the total weight of the particle. In some embodiments, the IR absorbing agent is present in an amount ranging from about 10.0 wt. % to about 13.0 wt. % by the total weight of the particle. In some embodiments, the IR absorbing agent is present in an amount ranging from about 10.5 wt. % to about 13.0 wt. % by the total weight of the particle. In some embodiments, the IR absorbing agent is present in an amount ranging from about 11.0 wt. % to about 13.0 wt. % by the total weight of the particle. In some embodiments, the IR absorbing agent is present in an amount ranging from about 11.5 wt. % to about 13.0 wt. % by the total weight of the particle. In some embodiments, the IR absorbing agent is present in an amount ranging from about 12.0 wt. % to about 13.0 wt. % by the total weight of the particle.

In some embodiments, the IR absorbing agent is present in an amount ranging from about 5.0 wt. % to about 12.0 wt. % by the total weight of the particle. In some embodiments, the IR absorbing agent is present in an amount ranging from about 5.5 wt. % to about 12.0 wt. % by the total weight of the particle. In some embodiments, the IR absorbing agent is present in an amount ranging from about 6.0 wt. % to about 12.0 wt. % by the total weight of the particle. In some embodiments, the IR absorbing agent is present in an amount ranging from about 6.5 wt. % to about 12.0 wt. % by the total weight of the particle. In some embodiments, the IR absorbing agent is present in an amount ranging from about 7.0 wt. % to about 12.0 wt. % by the total weight of the particle. In some embodiments, the IR absorbing agent is present in an amount ranging from about 7.5 wt. % to about 12.0 wt. % by the total weight of the particle. In some embodiments, the IR absorbing agent is present in an amount ranging from about 8.0 wt. % to about 12.0 wt. % by the total weight of the particle. In some embodiments, the IR absorbing agent is present in an amount ranging from about 8.5 wt. % to about 12.0 wt. % by the total weight of the particle. In some embodiments, the IR absorbing agent is present in an amount ranging from about 9.0 wt. % to about 12.0 wt. % by the total weight of the particle. In some embodiments, the IR absorbing agent is present in an amount ranging from about 9.5 wt. % to about 12.0 wt. % by the total weight of the particle. In some embodiments, the IR absorbing agent is present in an amount ranging from about 10.0 wt. % to about 12.0 wt. % by the total weight of the particle. In some embodiments, the IR absorbing agent is present in an amount ranging from about 10.5 wt. % to about 12.0 wt. % by the total weight of the particle. In some embodiments, the IR absorbing agent is present in an amount ranging from about 11.0 wt. % to about 12.0 wt. % by the total weight of the particle. In some embodiments, the IR absorbing agent is present in an amount ranging from about 11.5 wt. % to about 12.0 wt. % by the total weight of the particle.

In some embodiments, the IR absorbing agent is present in an amount ranging from about 5.0 wt. % to about 11.0 wt. % by the total weight of the particle. In some embodiments, the IR absorbing agent is present in an amount ranging from about 5.5 wt. % to about 11.0 wt. % by the total weight of the particle. In some embodiments, the IR absorbing agent is present in an amount ranging from about 6.0 wt. % to about 11.0 wt. % by the total weight of the particle. In some embodiments, the IR absorbing agent is present in an amount ranging from about 6.5 wt. % to about 11.0 wt. % by the total weight of the particle. In some embodiments, the IR absorbing agent is present in an amount ranging from about 7.0 wt. % to about 11.0 wt. % by the total weight of the particle. In some embodiments, the IR absorbing agent is present in an amount ranging from about 7.5 wt. % to about 11.0 wt. % by the total weight of the particle. In some embodiments, the IR absorbing agent is present in an amount ranging from about 8.0 wt. % to about 11.0 wt. % by the total weight of the particle. In some embodiments, the IR absorbing agent is present in an amount ranging from about 8.5 wt. % to about 11.0 wt. % by the total weight of the particle. In some embodiments, the IR absorbing agent is present in an amount ranging from about 9.0 wt. % to about 11.0 wt. % by the total weight of the particle. In some embodiments, the IR absorbing agent is present in an amount ranging from about 9.5 wt. % to about 11.0 wt. % by the total weight of the particle. In some embodiments, the IR absorbing agent is present in an amount ranging from about 10.0 wt. % to about 11.0 wt. % by the total weight of the particle. In some embodiments, the IR absorbing agent is present in an amount ranging from about 10.5 wt. % to about 11.0 wt. % by the total weight of the particle.

In some embodiments, the IR absorbing agent is present in an amount ranging from about 5.0 wt. % to about 10.0 wt. % by the total weight of the particle. In some embodiments, the IR absorbing agent is present in an amount ranging from about 5.5 wt. % to about 10.0 wt. % by the total weight of the particle. In some embodiments, the IR absorbing agent is present in an amount ranging from about 6.0 wt. % to about 10.0 wt. % by the total weight of the particle. In some embodiments, the IR absorbing agent is present in an amount ranging from about 6.5 wt. % to about 10.0 wt. % by the total weight of the particle. In some embodiments, the IR absorbing agent is present in an amount ranging from about 7.0 wt. % to about 10.0 wt. % by the total weight of the particle. In some embodiments, the IR absorbing agent is present in an amount ranging from about 7.5 wt. % to about 10.0 wt. % by the total weight of the particle. In some embodiments, the IR absorbing agent is present in an amount ranging from about 8.0 wt. % to about 10.0 wt. % by the total weight of the particle. In some embodiments, the IR absorbing agent is present in an amount ranging from about 8.5 wt. % to about 10.0 wt. % by the total weight of the particle. In some embodiments, the IR absorbing agent is present in an amount ranging from about 9.0 wt. % to about 10.0 wt. % by the total weight of the particle.

In some embodiments, the IR absorbing agent is present in an amount ranging from about 8.0 wt. % to about 10.0 wt. % by the total weight of the particle. In some embodiments, the IR absorbing agent is present in an amount ranging from about 7.0 wt. % to about 10.0 wt. % by the total weight of the particle. In some embodiments, the IR absorbing agent is present in an amount ranging from about 6.0 wt. % to about 10.0 wt. % by the total weight of the particle. In some embodiments, the IR absorbing agent is present in an amount ranging from about 5.0 wt. % to about 10.0 wt. % by the total weight of the particle. In some embodiments, the IR absorbing agent is present in an amount ranging from about 4.0 wt. % to about 10.0 wt. % by the total weight of the particle. In some embodiments, the IR absorbing agent is present in an amount ranging from about 3.0 wt. % to about 10.0 wt. % by the total weight of the particle.

In some embodiments, the particles comprise IR absorbing agent in an amount selected from the group of about 5.0 wt. %, about 5.56 wt. %, about 10.4 wt. %, about 12.0 wt. %, about 12.1 wt. %, about 13.64 wt. %, about 14.0 wt. %, or about 15.0 wt. % by the total weight of the particles. In some embodiments, the particles comprise IR absorbing agent in an amount of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, or about 15.0 wt. %.

In some embodiments, the carrier is formed of polymer or co-polymers; examples include but may not limited to polycarbonate polyacrylates, polymethacrylates and copolymers thereof, polyurethanes, polyureas, cellulosic materials, polymaleic acid and its derivatives, and polyvinyl acetate. In some embodiments, the carrier comprises polymethacrylates and copolymers thereof.

In some embodiments, the polymer carrier has a glass transition temperature (T_{g}) of at least 45° C. In some embodiments, the polymer carrier has a glass transition temperature ranging from 45° C. to 120° C. In some embodiments, the polymer carrier has a glass transition temperature ranging from 45° C. to 100° C. In some embodiments, the polymer carrier has a glass transition temperature ranging from 55° C. to 100° C. In some embodiments, the polymer carrier has a glass transition temperature ranging from 75° C. to 100° C. In some embodiments, the polymer carrier has a glass transition temperature ranging from 95° C. to 100° C. In some embodiments, the polymer carrier has a glass transition temperature selected from the group of 45° C., 50° C., 55° C., 60° C., 65° C., 70° C., 75° C., 80° C., 85° C., 90° C., 95° C., 100° C., 110° C., or 120° C. In some embodiments, the polymer carrier has a glass transition temperature is selected from the group of 95° C., 96° C., 97° C., 98° C., 99° C., or 100° C. In some embodiments, the polymer carrier has a glass transition temperature at 99° C. It is preferred that the polymer T_{g} be greater than about 37° C.

In one embodiment, the polymeric carrier is polymethylmethacrylate (PMMA). In some embodiments, the polymeric carrier is a polyacrylate blend comprising 96% polymethylmethacrylate and 4% polybutylacrylate. In some embodiments, the polymer carrier is a polymethacrylate /butylacrylate copolymer comprising 96% methylmethacrylate repeating units and 4% butylacrylate repeating units. In some embodiments, the polymethyl methacrylate is a copolymer of methylmethacrylate/butylacrylate (NeoCryl® B-805, T_{g} 99° C., average molecular weight 85,000 Da).

In some embodiments, the particle comprises NeoCryl® B-805 (copolymer of 96.0 wt. % methylmethacrylate/4.0 wt. % butylacrylate) in an amount ranging from about 60.0 wt. % to about 85 wt. % by the total weight of the particle. In some embodiments, the particle comprises NeoCryl® B-805 in an amount ranging from about 65.0 wt. % to about 85 wt. % by the total weight of the particle. In some embodiments, the particle comprises NeoCryl® B-805 in an amount ranging from about 70.0 wt. % to about 85 wt. % by the total weight of the particle. In some embodiments, the particle comprises NeoCryl® B-805 in an amount ranging from about 71.0 wt. % to about 85 wt. % by the total weight of the particle. In some embodiments, the particle comprises NeoCryl® B-805 in an amount ranging from about 72.0 wt. % to about 85 wt. % by the total weight of the particle. In some embodiments, the particle comprises NeoCryl® B-805 in an amount ranging from about 72.5 wt. % to about 85 wt. % by the total weight of the particle. In some embodiments, the particle comprises NeoCryl® B-805 in an amount ranging from about 73.0 wt. % to about 85 wt. % by the total weight of the particle. In some embodiments, the particle comprises NeoCryl® B-805 in an amount ranging from about 74.0 wt. % to about 85 wt. % by the total weight of the particle. In some embodiments, the particle comprises NeoCryl® B-805 in an amount ranging from about 75.0 wt. % to about 85 wt. % by the total weight of the particle. In some embodiments, the particle comprises NeoCryl® B-805 in an amount ranging from about 76.0 wt. % to about 85 wt. % by the total weight of the particle. In some embodiments, the particle comprises NeoCryl® B-805 in an amount ranging from about 77.0 wt. % to about 85 wt. % by the total weight of the particle. In some embodiments, the particle comprises NeoCryl® B-805 in an amount ranging from about 78.0 wt. % to about 85 wt. % by the total weight of the particle. In some embodiments, the particle comprises NeoCryl® B-805 in an amount ranging from about 79.0 wt. % to about 85 wt. % by the total weight of the particle. In some embodiments, the particle comprises NeoCryl® B-805 in an amount ranging from about 80.0 wt. % to about 85 wt. % by the total weight of the particle.

In some embodiments, the particle comprises NeoCryl® B-805 in an amount ranging from about 65.0 wt. % to about 80 wt. % by the total weight of the particle. In some embodiments, the particle comprises NeoCryl® B-805 in an amount ranging from about 64.0 wt. % to about 80 wt. % by the total weight of the particle. In some embodiments, the particle comprises NeoCryl® B-805 in an amount ranging from about 63.0 wt. % to about 80 wt. % by the total weight of the particle. In some embodiments, the particle comprises NeoCryl® B-805 in an amount ranging from about 62.0 wt. % to about 80 wt. % by the total weight of the particle. In some embodiments, the particle comprises NeoCryl® B-805 in an amount ranging from about 61.0 wt. % to about 80 wt. % by the total weight of the particle. In some embodiments, the particle comprises NeoCryl® B-805 in an amount ranging from about 60.0 wt. % to about 80 wt. % by the total weight of the particle. In some embodiments, the particle comprises NeoCryl® B-805 in an amount ranging from about 59.0 wt. % to about 80 wt. % by the total weight of the particle. In some embodiments, the particle comprises NeoCryl® B-805 in an amount ranging from about 58.0 wt. % to about 80 wt. % by the total weight of the particle. In some embodiments, the particle comprises NeoCryl® B-805 in an amount ranging from about 57.0 wt. % to about 80 wt. % by the total weight of the particle. In some embodiments, the particle comprises NeoCryl® B-805 in an amount ranging from about 56.0 wt. % to about 80 wt. % by the total weight of the particle. In some embodiments, the particle comprises NeoCryl® B-805 in an amount ranging from about 55.0 wt. % to about 80 wt. % by the total weight of the particle. In some embodiments, the particle comprises NeoCryl® B-805 in an amount ranging from about 55.0 wt. % to about 85 wt. % by the total weight of the particle. In some embodiments, the particle comprises NeoCryl® B-805 in an amount ranging from about 56.0 wt. % to about 84 wt. % by the total weight of the particle. In some embodiments, the particle comprises NeoCryl® B-805 in an amount ranging from about 57.0 wt. % to about 83 wt. % by the total weight of the particle. In some embodiments, the particle comprises NeoCryl® B-805 in an amount ranging from about 58.0 wt. % to about 82 wt. % by the total weight of the particle. In some embodiments, the particle comprises NeoCryl® B-805 in an amount ranging from about 59.0 wt. % to about 81 wt. % by the total weight of the particle. In some embodiments, the particle comprises NeoCryl® B-805 in an amount ranging from about 60.0 wt. % to about 80 wt. % by the total weight of the particle. In some embodiments, the particle comprises NeoCryl® B-805 in an amount ranging from about 61.0 wt. % to about 79 wt. % by the total weight of the particle. In some embodiments, the particle comprises NeoCryl® B-805 in an amount ranging from about 61.0 wt. % to about 78 wt. % by the total weight of the particle

In some embodiments, the particle comprises NeoCryl® B-805 in an amount ranging from about 62.0 wt. % to about 64.0 wt. % by the total weight of the particle. In some embodiments, the particle comprises NeoCryl® B-805 in an amount ranging from about 62.0 wt. % to about 74 wt. % by the total weight of the particle. In some embodiments, the particle comprises NeoCryl® B-805 in an amount ranging from about 61.0 wt. % to about 77 wt. % by the total weight of the particle. In some embodiments, the particle comprises NeoCryl® B-805 in an amount ranging from about 61.0 wt. % to about 76 wt. % by the total weight of the particle. In some embodiments, the particle comprises NeoCryl® B-805 in an amount ranging from about 61.0 wt. % to about 75 wt. % by the total weight of the particle. In some embodiments, the particle comprises NeoCryl® B-805 in an amount ranging from about 61.0 wt. % to about 74 wt. % by the total weight of the particle.

In some embodiments, the particle comprises NeoCryl® B-805 in an amount ranging from about 70.0 wt. % to about 80.0 wt. % by the total weight of the particle. In some embodiments, the particle comprises NeoCryl® B-805 in an amount ranging from about 71.0 wt. % to about 79 wt. % by the total weight of the particle. In some embodiments, the particle comprises NeoCryl® B-805 in an amount ranging from about 72.0 wt. % to about 78 wt. % by the total weight of the particle. In some embodiments, the particle comprises NeoCryl® B-805 in an amount ranging from about 72.0 wt. % to about 77 wt. % by the total weight of the particle. In some embodiments, the particle comprises NeoCryl® B-805 in an amount ranging from about 72.0 wt. % to about 76 wt. % by the total weight of the particle. In some embodiments, the particle comprises NeoCryl® B-805 in an amount ranging from about 72.0 wt. % to about 75 wt. % by the total weight of the particle.

In some embodiments, the particle comprises NeoCryl® B-805 in an amount selected from the group of 62.0 wt. %, 70.0 wt. %, 75.0 wt. % or 78.3 wt. % by the total weight of the particle. In some embodiments, the particle comprises NeoCryl® B-805 in an amount selected from the group of about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, or about 80 wt. % by the total weight of the particle.

In some embodiments, the particle containing the NeoCryl® B-805 polymer carrier and the dye has a weight ratio of the NeoCryl® B-805 polymer carrier to the dye ranging from 1:1 to 7:1 (see Table 3 below). In some embodiments, the particle containing the NeoCryl® B-805 polymer carrier and the dye has a weight ratio of the NeoCryl® B-805 polymer carrier to the dye ranging from 2:1 to 7:1. In some embodiments, the particle containing the NeoCryl® B-805 polymer carrier and the dye has a weight ratio of the NeoCryl® B-805 polymer carrier to the dye ranging from 3:1 to 7:1. In some embodiments, the particle containing the NeoCryl® B-805 polymer carrier and the dye has a weight ratio of the polymer carrier to the dye ranging from 5:1 to 7:1. In some embodiments, the particle containing the NeoCryl® B-805 polymer carrier and the dye has a weight ratio of the polymer carrier to the dye selected from the group of 1:1, 2:1, 3:1, 4:1, 5:1, 6:1, or 7:1. In some embodiments, the particle containing the NeoCryl® B-805 polymer carrier and the dye has a weight ratio of the NeoCryl® B-805 polymer carrier to the dye is 3:1. In some embodiments, the particle containing the NeoCryl® B-805 polymer carrier and the dye has a weight ratio of the NeoCryl® B-805 polymer carrier to the dye is 4:1. In some embodiments, the particle containing the NeoCryl® B-805 polymer carrier and the dye has a weight ratio of the NeoCryl® B-805 polymer carrier to the dye is 5:1. In some embodiments, the particle containing the NeoCryl® B-805 polymer carrier and the dye has a weight ratio of the NeoCryl® B-805 polymer carrier to the dye is 6:1. In some embodiments, the particle containing the NeoCryl® B-805 polymer carrier and the dye has a weight ratio of the NeoCryl® B-805 polymer carrier to the dye is 7:1.

In some embodiments, the shell comprises a cross-linked polymeric structure. In some embodiments, the cross-linked polymer is an inorganic polymer. In some embodiments, the inorganic polymer is a sol-gel organo-modified silicone polymer prepared by Stöber reaction. In some embodiments, the shell comprises sol-gel vinylsilicate made from VTMS reactant by Stöber reaction. In some embodiments, the particle shell further has a surface modification. In some embodiments, the surface modification on the shell comprises a hydrophilic polymer coating on the shell.

In some embodiments, the core-shell particles have approximately spherical shape. In some embodiments, the particles are microparticles have a median particle size selected from the group of 0.5 μm, 0.7 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10.0 μm, 11.0 μm, 12.0 μm, 13.0 μm, 14.0 μm, 15.0 μm, 16.0 μm, 17.0 μm, 18.0 μm, 19.0 μm, 20.0 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, 150 μm, 155 μm, or 160 μm. In some embodiments, the particles are microparticles have a median particle size selected from the group of 0.5 μm, 0.7 μm, 1.0 μm, 2.0 μm, 3.0 μm, 4.0 μm, 5.0 μm, or 6.0 μm. In some embodiments, the core-shell particles are microparticles have a median particle size in a range from about 1 μm to about 10 μm. In some embodiments, the core-shell particles are microparticles have a median particle size in a range from about 1 μm to about 9 μm. In some embodiments, the core-shell particles are microparticles have a median particle size in a range from about 1 μm to about 8 μm. In some embodiments, the core-shell particles are microparticles have a median particle size in a range from about 1 μm to about 7 μm. In some embodiments, the core-shell particles are microparticles have a median particle size in a range from about 1 μm to about 6 μm. In some embodiments, the core-shell particles are microparticles have a median particle size in a range from about 1 μm to about 5 μm. In some embodiments, the core-shell particles are microparticles have a median particle size in a range from about 1 μm to about 4 μm. In some embodiments, the core-shell particles are microparticles have a median particle size in a range from about 1 μm to about 3 μm. In some embodiments, the core-shell particles are microparticles have a median particle size in a range from about 1 μm to about 2 μm. In some embodiments, the core-shell particles are microparticles have a median particle size in a range from about 2 μm to about 10 μm. In some embodiments, the core-shell particles are microparticles have a median particle size in a range from about 2 μm to about 9 μm. In some embodiments, the core-shell particles are microparticles have a median particle size in a range from about 2 μm to about 8 μm. In some embodiments, the core-shell particles are microparticles have a median particle size in a range from about 2 μm to about 7 μm. In some embodiments, the core-shell particles are microparticles have a median particle size in a range from about 2 μm to about 6 μm. In some embodiments, the core-shell particles are microparticles have a median particle size in a range from about 2 μm to about 5 μm. In some embodiments, the core-shell particles are microparticles have a median particle size in a range from about 2 μm to about 4 μm. In some embodiments, the core-shell particles are microparticles have a median particle size in a range from about 2 μm to about 3 μm. In some embodiments, the core-shell particles are microparticles have a median particle size in a range from about 3 μm to about 10 μm. In some embodiments, the core-shell particles are microparticles have a median particle size in a range from about 3 μm to about 9 μm. In some embodiments, the core-shell particles are microparticles have a median particle size in a range from about 3 μm to about 8 μm. In some embodiments, the core-shell particles are microparticles have a median particle size in a range from about 3 μm to about 7 μm. In some embodiments, the core-shell particles are microparticles have a median particle size in a range from about 3 μm to about 6 μm. In some embodiments, the core-shell particles are microparticles have a median particle size in a range from about 3 μm to about 5 μm. In some embodiments, the core-shell particles are microparticles have a median particle size in a range from about 3 μm to about 4 μm.

In some embodiments, the particles are microparticles have a median particle size in a range from about 10 μm to about 150 μm. In some embodiments, the microparticles have a median particle size in a range from about 1 μm to about 6 μm. In some embodiments, the microparticles have a median particle size in a range from about 1 μm to about 4 μm. For tattoo pigments, it is generally preferred that the particles have a median particle size between about 0.5 μm to about 5 μm.

Using conventional, linear or modestly branched polymers as the carrier, it has been found that the free volume or porosity of the carrier can allow an unacceptable amount of leakage, as determined by the Extractable Cytotoxicity Test. As a result, it has been found that coating the initially formed particle with a cross-linked inorganic polymer shell improves the resistance of the particle to incursion by biological media. The degree of cross-linking of the shell affects the shell porosity, and consequentially, reducing the porosity of the shell by increasing cross-linking improves particle performance in the ECT to achieve IC₃₀ or less. The shell may comprise inorganic polymers such as silicates, organosilicate, organo-modified silicone polymer, or may be cross-linked organic polymers such as polyureas or polyurethanes. The process to apply the cross-linked shell must be designed so as to maximize the stability of the particle components to the chemistry required in shell construction, at least until the growing shell protects the components encapsulated in the particle.

In an embodiment, the particle suitable for tissue marking applications (e.g., tattoo ink) comprises a core containing (i) about 21.6 wt. % to about 38.0 wt. % of a colorant, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (EpolightTM 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.083:1 to 0.66:1 (7.5 wt. % VTMS to 40 wt. % VTMS by the weight of VTMS reagent and uncoated particle); wherein the particle having median particle size of 0.5 μm, 0.7 μm, 1 μm, 2 μm, 3 μm, 4 μm, 5 μm, or 6 μm. When the weight ratio of the VTMS reagent to the uncoated particle is 0.083:1, the weight amount of VTMS applied in relating to the core-shell particle is 7.5 wt. % by the total weight of the VTMS reagent and uncoated particle. When the weight ratio of the VTMS reagent to the uncoated particle is 0.33:1, the weight amount of VTMS applied in relating to the uncoated particle is 25 wt. % by the total weight of the VTMS reagent and uncoated particle. When the weight ratio of the VTMS reagent to the uncoated particle is 0.66:1, the weight amount of VTMS applied in relating to the uncoated particle is 40 wt. % by the total weight of the VTMS reagent and uncoated particle.

In an embodiment, the particle suitable for tissue marking applications (e.g., tattoo ink) comprises a core containing (i) about 21.6 wt. % to about 38.0 wt. % of a colorant, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (EpolightTM 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.33:1 to 0.66:1 (25 wt. % VTMS to 40 wt. % VTMS by the weight of VTMS reagent and uncoated particle), wherein the particle has a median particle size of 0.5 μm, 0.7 μm, 1 μm, 2 μm, 3 μm, 4 μm, 5 μm, or 6 μm.

In an embodiment, the particle suitable for tissue marking applications (e.g., tattoo ink) comprises a core containing (i) about 21.6 wt. % to about 38.0 wt. % of a colored compound having formula D-Sp-Nu-FG as described above, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (Epolight™ 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.33:1 to 0.66:1 (25 wt. % VTMS to 40 wt. % VTMS by the weight of VTMS reagent and uncoated particle), wherein the particle has a median particle size of 0.5 μm, 0.7 μm, 1 μm, 2 μm, 3 μm, 4 μm, 5 μm, or 6 μm.

In an embodiment, the particle suitable for tissue marking applications (e.g., tattoo ink) comprises a core containing (i) about 21.6 wt. % to about 38.0 wt. % of a colored compound selected from compounds having formulas (I)-(VII) as described above, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (Epolight™ 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.33:1 to 0.66:1 (25 wt. % VTMS to 40 wt. % VTMS by the weight of VTMS reagent and uncoated particle), wherein the particle has a median particle size of 0.5 μm, 0.7 μm, 1 μm, 2 μm, 3 μm, 4 μm, 5 μm, or 6 μm.

In an embodiment, the particle suitable for tissue marking applications (e.g., tattoo ink) comprises a core containing (i) about 21.6 wt. % to about 38.0 wt. % of a colorant, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (EpolightTM 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.083:1 to 0.33:1 (7.5 wt. % VTMS to 25 wt. % VTMS by the weight of VTMS reagent and uncoated particle), wherein the particle has a median particle size of 0.5 μm, 0.7 μm, 1 μm, 2 μm, 3 μm, 4 μm, 5 μm, or 6 μm.

In an embodiment, the particle suitable for tissue marking applications (e.g., tattoo ink) comprises a core containing (i) about 21.6 wt. % to about 38.0 wt. % of a colorant, (ii) about 62.0 wt. % to about 78.3 wt. % copolymer of methyl methacrylate and butyl methacrylate 96:4 (Neocryl® 805); (iii) about 5.56 wt. % to about 14.0 wt. % IR absorbing agent (EpolightTM 1117); and a sol gel vinyl modified silicate polymer shell made from a hydrolyzed vinyltrimethoxysilane (VTMS) solution to enclose the core, in which the weight ratio of the VTMS reagent to the uncoated particle is 0.33:1 (25 wt. % VTMS by the weight of VTMS reagent and uncoated particle), wherein the particle has a median particle size of 0.5 μm, 0.7 μm, 1 μm, 2 μm, 3 μm, 4 μm, 5 μm or 6 μm.

In an embodiment, the particle suitable for tissue marking applications (e.g., tattoo ink) comprises a core containing (i) about 38.0 wt. % of a colorant, (ii) about 62.0 wt. % copolymer of methyl methacrylate and butyl methacrylate 96:4 (Neocryl® 805); (iii) about 5.56 wt. % to about 14.0 wt. % IR absorbing agent (Epolight™ 1117); and a sol gel vinyl modified silicate polymer shell made from a hydrolyzed vinyltrimethoxysilane (VTMS) solution to enclose the core, in which the weight ratio of the VTMS reagent to the uncoated particle is from 0.33:1 (25 wt. % VTMS by the weight of VTMS reagent and uncoated particle), wherein the particle has a median particle size of 2 μm.

In an embodiment, the particle suitable for tissue marking applications (e.g., tattoo ink) comprises a core containing (i) about 25.0 wt. % of a colorant, (ii) about 75.0 wt. % copolymer of methyl methacrylate and butyl methacrylate 96:4 (Neocryl® 805); (iii) about 5.56 wt. % to about 14.0 wt. % IR absorbing agent (Epolight™ 1117); and a sol gel vinyl modified silicate polymer shell made from a hydrolyzed vinyltrimethoxysilane (VTMS) solution to enclose the core, in which the weight ratio of the VTMS reagent to the uncoated particle is 0.33:1 (25 wt. % VTMS by the weight of VTMS reagent and uncoated particle), wherein the particle has a median particle size of 0.5 μm, 0.7 μm, 1 μm, 2 μm, or 3 μm.

In an embodiment, the particle suitable for tissue marking applications (e.g., tattoo ink) comprises a core containing (i) about 21.7 wt. % of a colorant, (ii) about 78.3 wt. % copolymer of methyl methacrylate and butyl methacrylate 96:4 (Neocryl® 805); (iii) about 5.56 wt. % to about 14.0 wt. % IR absorbing agent (Epolight™ 1117); and a sol gel vinyl modified silicate polymer shell made from a hydrolyzed vinyltrimethoxysilane (VTMS) solution to enclose the core, in which the weight ratio of the VTMS reagent to the uncoated particle is 0.33:1 (25 wt. % VTMS by the weight of VTMS reagent and uncoated particle), wherein the particle has a median particle size of 1 μm, 2 μm, or 3 μm.

The wt. % for ingredients of the particle are calculated based on the total weight of the particle without the shell. This calculation of the wt. % for ingredients of the particle is applicable to all wt. % of the particle ingredients disclosed above in this disclosure.

3. Additional Embodiments

In some embodiments, this disclosure provides a particle comprising (a) a core comprising a carrier, a material, and a colorant; and (b) a shell enclosing the core; wherein the shell comprises a cross-linked organo-silicate polymer derived from trialkoxysilane, or trihalorosilane; wherein the colorant and the material in the particle exhibit stability such that the particle is considered passing the Efficacy Determination Protocol; and wherein the particle structure is constructed such that it passes the Extractable Cytotoxicity Test.

In some embodiments, the trialkoxysilane used for making the shell is selected from the group of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group of trichlorosilane, tribromosilane, triiodosilane, and combinations thereof. In some embodiments, the cross-linked organo-silicate polymer is derived from vinyl-trimethoxysilane.

In some embodiments, the material absorbs the infrared radiation having wavelengths in the range of 700 to 1500 nm. In some embodiments, the material for absorbing infrared radiation is a tetrakis aminium dye. In some embodiments, the material for absorbing infrared radiation is a zinc iron phosphate pigment.

In some embodiments, the colorant is a visible colorant comprising a chromophore group and a thermally activatable fragmentation group, wherein the visible colorant is transformed to a colorless dye when the material absorbs radiation at infrared wavelengths. In some embodiments, the chromophore group is selected from the group of a substituted or unsubstituted triarylmethane, a xanthene, a rhodamine, a fluoran, an azocarbocyanine, a benzilidine, a thiazine, an acridine, an aminoanthraquinone, and combinations thereof. In some embodiments, the thermally activatable fragmentation group produces a nucleophilic group upon activation. In some embodiments, the thermally activatable fragmentation group is selected from the group of substituted and unsubstituted carbonates, carbamates, esters, lactams, lactones, amides, imides, oximes, sulfonates, phosphonates, and combinations thereof.

In some embodiments, this disclosure provides a particle comprising (a) a core comprising a carrier, a material, and a colorant composed of a chromophore group and a thermally activatable fragmentation group; and (b) a shell enclosing the core; wherein the colorant is transformed to colorless when the material absorbs radiation at infrared wavelengths; wherein the thermally activatable fragmentation group is selected from the group of substituted and unsubstituted carbonates, carbamates, esters, lactams, lactones, amides, imides, oximes, sulfonates, phosphonates, and combinations thereof; wherein the chromophore group is a xanthene dye and the thermally activatable fragmentation group produces a nucleophilic group upon activation; wherein the shell comprises a cross-linked organo-silicate polymer derived from trialkoxysilane, or trihalorosilane; wherein the colorant and the material in the particle exhibit stability such that the particle is considered passing the Efficacy Determination Protocol; and wherein the particle structure is constructed such that it passes the Extractable Cytotoxicity Test.

In some embodiments, the trialkoxysilane used to form the shell is selected from the group of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used to form the shell is selected from the group of trichlorosilane, tribromosilane, triiodosilane, and combinations thereof. In some embodiments, the cross-linked organo-silicate polymer is derived from vinyl-trimethoxysilane.

### 4. Tattoo Ink and Method of Making Permanent Tattoos

In some embodiments, this disclosure provides the use of the tattoo particles as described herein in making tattoo ink (also known as tissue marking) for the formation of permanent removable tattoo. The tattoo particles are mixed with a dermatologically acceptable liquid carrier to form tattoo inks with various colors including black, blue, yellow, green, magenta, cyan, neutral black (NB) and process black (PB). In some embodiments, the mixture of the tattoo particles and the liquid carrier forms an injectable liquid suspension suitable for use with conventional tattoo machines.

In some embodiments, the dermatologically acceptable liquid carrier is selected from the group of purified water, witch hazel, Listerine® mouthwash, and buffer solution.

In some embodiments, the dermatologically acceptable liquid carrier comprises Listerine® mouthwash, a liquid formulation containing essential oils menthol (mint) 0.042%, thymol (thyme) 0.064%, methyl salicylate (wintergreen) 0.06%, and eucalyptol (eucalyptus) 0.092%. In some embodiments, the dermatologically acceptable liquid carrier comprises witch hazel. In some embodiments, the dermatologically acceptable liquid carrier comprises purified water.

In some embodiments, the dermatologically acceptable liquid carrier comprises buffer solutions. In some embodiments, the buffer solutions comprise hydrogen ion buffers having pH between 6 and 8 (also known as Good's buffer). In some embodiments, the liquid carrier is a hydrogen ion buffer selected from the buffers listed in Table A below.

TABLE A
Good's Buffer

| | | | Solubility |
|---|---|---|---|
| | pKₐ at | ΔpKₐ/ | in water |
| Buffer | 20° C. | ° C. | at 0° C. |
| MES (2-(N-morpholino) | 6.15 | −0.011 | 0.65M |
| ethanesulfonic acid) | | | |
| (2-[Bis(2-hydroxyethyl)amino]-2- | 6.60 | | |
| (hydroxymethyl)propane-1,3-diol) | | | |
| ADA (2-[(2-amino-2-oxoethyl)- | 6.62 | −0.011 | — |
| (carboxymethyl)amino]acetic acid) | | | |
| ACES (N-(2-acetamido)-2- | 6.76 | | |
| aminoethanesulfonic acid) | | | |
| Bis-tris propane (1,3- | 6.80 | | — |
| bis(tris(hydroxymethyl) | | | |
| methylamino)propane) | | | |
| PIPES (piperazine-N,N′-bis(2- | 6.82 | −0.0085 | — |
| ethanesulfonic acid)) | | | |
| ACES (N-(2-acetamido)-2- | 6.88 | −0.020 | 0.22M |
| aminoethanesulfonic acid.) | | | |
| MOPSO (2-hydroxy-3-morpholin-4- | 6.95 | −0.015 | 0.75M |
| ylpropane-1-sulfonic acid) | | | |
| Cholamine chloride | 7.10 | −0.027 | 4.2M |
| | | | (as HCl) |
| MOPS ((3-(N-morpholino) | 7.15 | −0.013 | 3.0M |
| propanesulfonic acid)) | | | |
| BES | 7.17 | −0.016 | 3.2M |
| (OH—CH—CH₂)₂—⁺NH(CH₂CH₂SO₃⁻) | | | |
| TES (2-[[1,3-dihydroxy-2- | 7.5 | −0.020 | 2.6M |
| (hydroxymethyl)propan-2- | | | |
| yl]amino]ethanesulfonic acid) | | | |
| HEPES ((4-(2-hydroxyethyl)-1- | 7.55 | −0.014 | 2.25M |
| piperazineethanesulfonic acid)) | | | |
| DIPSO | 7.6 | −0.015 | 0.24M |
| MOBS | 7.6 | | |
| Acetamidoglycine | 7.7 | — | very high |
| TAPSO (3-[[1,3-dihydroxy-2- | 7.6 | −0.018 | 1.0M |
| (hydroxymethyl)propan-2-yl]amino]-2- | | | |
| hydroxypropane-1-sulfonic acid) | | | |
| TEA (triethanolamine) | 7.8 | | |
| POPSO | 7.85 | −0.013 | — |
| HEPPSO | 7.9 | −0.01 | 2.2M |
| EPS | 8.0 | | |
| HEPPS (3-[4-(2-Hydroxyethyl) | 8.1 | −0.015 | Large |
| piperazin-1-yl]propane-1-sulfonic acid) | | | |
| Tricine (N-(2-Hydroxy-1,1- | 8.15 | −0.021 | 0.8M |
| bis(hydroxymethyl)ethyl)glycine) | | | |
| Tris (tris(hydroxymethyl)aminomethane) | 8.2 | | |
| Glycinamide | 8.2 | −0.029 | 6.4M |
| | | | (as HCl) |
| Glycylglycine | 8.2 | | |
| HEPBS | 8.3 | | |
| Bicine | 8.35 | −0.018 | 1.1M |
| TAPS | 8.55 | −0.027 | Large |
| AMPB | 8.8 | | |
| CHES | 9.3 | | |
| AMP | 9.7 | | |
| AMPSO | 9.0 | | |
| CAPSO | 9.6 | | |
| CAPS | 10.4 | | |
| CABS | 10.7 | | |

In some embodiments, this disclosure provides a method of making a permanent tattoo in a subject comprising the step of injecting any of the tattoo ink as described above to an area of the skin of a subject to form the permanent tattoo on the subject; wherein the permanent tattoo has a first color imparted by the colorant, and wherein the first color changes to a second color when the permanent tattoo is exposed to a sufficient dose of laser at an infrared wavelength. In some embodiments, the second color is colorless. In some embodiments, the second color has a different hue from that of the first color.

In some embodiments, this disclosure provides a method of remotely triggering color-change from permanent tattoo comprising the step of administering a dose of laser at an infrared wavelength to any of the herein described permanent tattoo on the subject having the first color; wherein the laser causes the first color changing to a second color. In some embodiments, the second color is colorless. In some embodiments, the second color has a different hue from that of the first color.

In some embodiments, for any one of the herein described method, the color-change step further comprises repeating the step of applying a dose of laser. In some embodiments, the laser is a pulsed laser light. In some embodiments, the laser pulse duration is in a range from milliseconds to femtoseconds, and the laser has an oscillation wavelength at 1064 nm. In some embodiments, the laser emits light at 808 nm. In some embodiments, the laser emits light at 805 nm. In some embodiments, the laser pulse duration is in a range from nanoseconds to picoseconds. In some embodiments, the laser pulse duration is a combination of nano- and picoseconds.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### General Procedures

The compositions of this invention may be made by various methods known in the art. Such methods include those of the following examples, as well as the methods specifically exemplified below. For clarity, the term “uncoated particle” refers to the core of a core-shell particle.

### Example 1

### Particle Fabrication

Reagents source: Chemical reagents sodium dodecyl sulfate (SDS), polyvinyl alcohol (PVA) were purchased from Aldrich; dyes B141, C161, M071, Y161 were prepared at Bambu Vault LLC; vinyltrimethoxysilane (VTMS) was purchased from Gelest, Inc. Neocryl® B-805 polymer (MMA/BMA copolymer, weight average molecular weight=85,000 Da, glass transition temperature T_{g}=99° C.) was purchased from DSM. Epolight™ 1117 (tetrakis aminium, absorbing at 800 nm-1071 nm, melting point: 185-188° C., soluble in acetone, methylethylketone and cyclohexanone) was purchased from Epolin Inc. Antioxidant Cyanox® 1790 (1,3,5-tris(4-tert-butyl-3-hydroxy-2,6-dimethyl benzyl)-1,3,5-triazine-2,4,6-(1H,3H,5H)-trione, CAS NUMBER 040601-76-1) was purchased from Cytec Industries Inc.

### Example 1a

### Uncoated Particle Synthesis Through Emulsion Method

This method results in a primary particle (no shell) wherein both the colorant (e.g., tattoo dyes) and the material (e.g., IR absorbing agent) are in solid state solution thereby ensuring high absorbance.

Abbreviations: n-BMA: n-butyl methacrylate; MMA: methyl-methacrylate

Preparation of the aqueous phase: 1.2 g of sodium dodecyl sulfate (SDS) was added into 190 g of 4.9% aqueous polyvinyl alcohol (PVA) solution placed in a round bottom flask. An aqueous solution of SDS containing 4.9% PVA was formed after the dissolution of SDS (the aqueous phase). The aqueous phase was stirred with an IKA t-25 Turrax at 8000 RPM.

The preparation of the organic phase: to 88 g of dichloromethane was added 8.0 g of DSM Neocryl® B-805 polymer (MMA/BMA copolymer), 1.19 g of B141 dye, 0.36 g of C161 dye, 0.36 g of M071 dye, 0.60 g of Y161 dye, 1.82 g of Epolight™ 1117 dye, and 0.65 g of Cyanox® 1790 to allow the formation of a clear solution of Neocryl® B805 polymer and dyes (the polymer +Cyanox®: dye weight ratio=2:1).

The organic phase (polymer and dyes dissolved in dichloromethane) was injected directly into the aqueous phase (PVA solution with SDS surfactant) at the tip of the Turrax's rotostator. The shear mixing at 8000 RPM was continued for 30 minutes. The resulting mixture was decanted into an open-mouth container and magnetically stirred for 16 hours. A suspension of solid black-dye particles in aqueous fluid was produced.

The suspension of particles was centrifuged at 5000 RPM for 30 minutes and the particles were collected. The collected particles were washed with distilled water by resuspending the particles into distilled water and centrifuging to collect the particles. This particle washing process was repeated three times to remove the residual PVA. The resulting dye/MMA/BMA copolymer particles were suspended in distilled water.

It will be seen by the data below that the uncoated particles (without shell) allowed for sufficient leakage of both the colorant and material so as to fail the Extractable Cytotoxicity Test. Examples in which a shell was created over the uncoated particles are described below.

Example lb. Synthesis of Dye Particles Having a 25% VTMS Shell

In this example, a sol-gel vinyl modified silicone polymer shell was made from a VTMS HC1 solution containing VTMS at 25 wt. % of the total weight of the VTMS HC1 solution. The weight amount of VTMS in the solution comprised 25 wt. % of the total weight of the VTMS reagent and uncoated particle (weight ratio VTMS/uncoated particle =0.33:1), hereafter referred to as the “25% VTMS shell”.

In a first vessel, 1.52 g (0.01 mmol) of vinyltrimethoxysilane (CH₂═CHSi(OMe)3, VTMS, MW=148 Da) was mixed with 4.58 g of dilute aqueous hydrochloric acid at a pH of 3.5 under magnetic stirring (24.9 wt. % solution of CH₂=CHSi(OMe)₃ in diluted HCl). The resulting mixture was stirred for 2 hours to allow complete hydrolysis of VTMS to give vinylsilanetriol (CH₂═CHSi(OH)₃, MW=106 Da).

In a second vessel, under magnetic stirring, 3 g of pre-made uncoated dye particles of Example 1a above were dispersed in 57 grams of water to provide a 5 wt. % dye particle dispersion. The pH value of the resulting dye particle aqueous dispersion was adjusted to 10.0 with the addition of dilute aqueous ammonium hydroxide. To this particle dispersion at pH 10, an aliquot of 3.99 g of the hydrolyzed 25 wt. % VTMS solution was added at a rate of 2 drops per second to the particle suspension. The pH value of the resulting suspension was monitored after the hydrolyzed 25% VTMS solution addition and adjusted with ammonium hydroxide solution to maintain a pH of 10 for 60 minutes. After 60 minutes, the suspension was neutralized with glacial acetic acid to lower the pH from 10 to 4.6-5.7. The weight ratio of VTMS to the uncoated particle was 0.33:1.

The resulting particle suspension was centrifuged for 30 minutes at 5000 RPM to collect the sol gel vinylsilicate-coated dye particles. The particles collected after the centrifugation were redispersed in distilled water and subjected to centrifugation to collect the particles. This washing procedure was repeated 3 times to remove any unreacted chemical reagents. The resulting sol gel vinylsilicate-coated particles were suspended in distilled water.

To tune the level of leakage of payloads (the colorant and/or the material) from the interior of the particle, the thickness of the sol-gel vinyl modified silicone polymer shell made from VTMS reagent in the Stober reaction was varied. The particles having different shell thicknesses can be prepared using the same procedure described above. If the amount of VTMS applied to form the shell was at the weight ratio of VTMS/uncoated particle of 0.1:1, the amount of VTMS applied was 9.1 wt. % by the total weight of the VTMS reagent and uncoated particle, hereafter referred to as the “9.1% VTMS shell”. If the amount of VTMS applied to form the shell was at the weight ratio of VTMS/uncoated particle of 0.66:1, the amount of VTMS applied was 40 wt. % by the total weight of the VTMS reagent and uncoated particle, hereafter referred to as the “40% VTMS shell”.

### Example 2

### Characterization of Particle Physicochemical Properties

2a. Particle Size Distribution

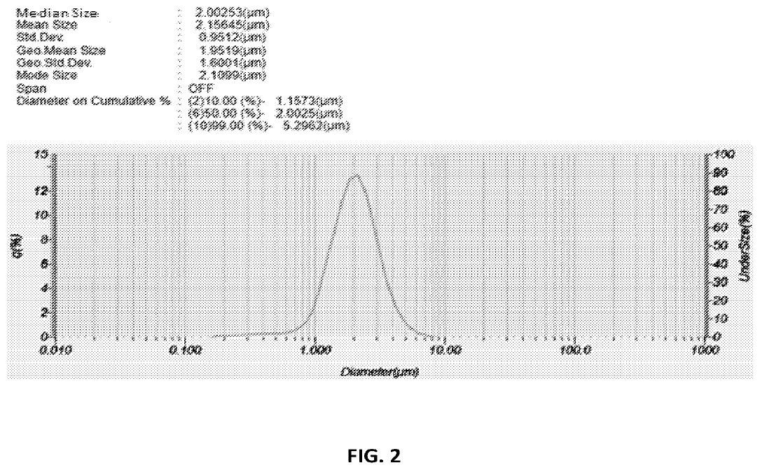

The particle size distribution of the resulting dye/MMA/BMA copolymer particles of Example 1b were measured with Horiba LA-950 Particle Size Analyzer in distilled water at pH 7.4 (FIG. 2). All particle size measurements were carried out at room temperature (about 17-22° C.). The median particle size (D₅₀) for the resulting black dye/MMA/BMA copolymer particles was 2.0 μm.

Various additional examples of dye particles were prepared according to the procedures set forth above. The physicochemical properties of the resulting particles are summarized in Table 3 below.

2b. The Dye Loading Determination

The particles were dried and ground in a mortar and pestle. An aliquot of 5-10 milligrams of the ground particles were added to 25 mL of dichloromethane (DCM). The absorbance spectrum of the extracted dye was measured over the range 400-1300 nm using a Shimadzu UV-3600 UV/VIS/NIR Spectrophotometer. The concentration of the extracted dye in DCM was determined from application of Beer's law (Eqn. 1) using the values given in Table 2.

$$[ Dye ] ( μM ) = \frac{{Absorbance}_{λ}}{{ε}_{λ} \times l} \times {10}^{6} \qquad ( Eqn . 1 )$$

where ε is taken from Table 2 and the path length, 1, is 1 cm, and dye include both colorant and IR absorbing agent.

TABLE 2
Spectral constants for visible and IR absorbing agents in particles

| | Extinction | Wavelength | Molecular |
|---|---|---|---|
| Dye | Coefficient (ϵ) | (λₘₐₓ) | weight |
| B141 | 18,600 M⁻¹ * cm⁻¹ | 606 nm | 759 g/mol |
| M071 | 85,000 M⁻¹ * cm⁻¹ | 558 nm | 792 g/mol |
| C161 | 70,000 M⁻¹ * cm⁻¹ | 680 nm | 747 g/mol |
| Y161 | 30,000 M⁻¹ * cm⁻¹ | 454 nm | 697 g/mol |
| IR1117 | 95,000 M⁻¹ * cm⁻¹ | 1064 nm | 1,211 g/mol |

The quantity of dye extracted was determined from the product of the concentration, the amount of total DCM solution (25 ml), and the molecular weight of the dye. Dye loading as a percentage of the total particle mass can be determined from Eqn 2.

$$Dye Loading ( \% ) = \frac{Amount of dye in DCM solution}{Amount of particle used} \times 100 \% \\ The Polymer / Dye weight ratio is then given as ( \frac{1}{Dye Loading} - 1 ) : 1 \qquad ( Eqn 2 )$$

TABLE 3
Color Particle Structure

| | | | Median | | |
|---|---|---|---|---|---|
| | Color | Polymer | Particle Size | Polymer/Dye | |
| Entry | Particle^{a} | Carrier | (micron) | Weight Ratio | Shell |
| 1 | NB | B805^{b} | 1, 3, 6 | 3:1 | — |
| 2 | NB | B805 | 3, 4 | 7:1 | — |
| 3 | NB | B805 | 3 | 3:1 | VTMS |
| 4 | PB1 | B805 | 0.5, 1, 3 | 3:1 | — |
| 5 | PB1 | B805 | 0.5, 0.7 | 3:1 | VTMS |
| 8 | PB1 with | B805 | 0.5, 1 | 3:1 | — |
| | Cyanox1790^{c} | | | | |
| 9 | PB1 with | B805 | 0.5, 1 | 3:1 | VTMS |
| | Cyanox1790 | | | | |
| 10 | PB1 | B728^{d} | 3 | 3:1 | — |
| 11 | PB1 with | B728 | 3 | 3:1 | — |
| | Cyanox1790 | | | | |
| 12 | PB2 with | B805 | 0.5, 0.7 | 3:1 | — |
| | Cyanox1790 | | | | |
| 13 | PB2 with | B805 | 0.5 | 3:1 | VTMS |
| | Cyanox1790 | | | | |
| 14 | PB3 with | B805 | 0.7, 1, 1.5, 2 | 3:1 | — |
| | Cyanox1790 | | | | |
| 15 | PB3 with | B805 | 0.7, 1, 1.5, 2 | 3:1 | VTMS |
| | Cyanox1790 | | | | |
| 16 | PB4 with | B805 | 0.5, 1, 1.5, 2, 3 | 2:1 | — |
| | Cyanox1790 | | | | |
| 17 | PB4 with | B805 | 0.5, 1, 1.5, 2, 3 | 2:1 | VTMS |
| | Cyanox1790 | | | | |
| 18 | Magenta | B805 | 1, 2, 3 | 5:1 | — |
| 19 | Cyan | B805 | 1, 2 | 5:1 | — |
| 20 | Yellow | B805 | 1, 2 | 5:1 | — |
| 21 | Yellow 197 | B805 | 2 | 5:1 | VTMS |
| 22 | M071 | B805 | 2 | 7:1 | VTMS |
| 23 | PB5 | B805 | 2 | 4:1 | VTMS |
| 24 | Y184 | B805 | 2 | 5:1 | VTMS |

^{a}Neutra Black (NB) and Process Black (PB) compositions as defined in Table 2.
^{b}Polymer B805: polyacrylate blend, 96% polymethylmethacrylate (PMMA) and 4% polybutylacrylate (Neocryl ® B-805 sold by DSM)
^{c}Cyanox1790: dye stabilizer mixed in the polymer matrix
^{d}Polymer 728: PMMA (Neocryl ® B-728 sold by DSM)

### Example 3

### Surfactant-Based Extractable Test

Absorbance spectra were measured from 400-1300 nm using Shimadzu UV-3600 UV-NIR Spectrophotometer. The absorbance spectrum of black dye B141 showed peaks at wavelength λ=464 nm and 606 nm which are the characteristics peaks for this dye molecule. Likewise, cyan dye C161 showed a characteristic maximum at λ=678 nm, magenta dye M071 showed a characteristic maximum at λ=558 nm, yellow dye Y161 showed a characteristic maximum at 454 nm, and the tetrakis aminium IR absorbing agent Epolight™ 1117 showed characteristic maxima at X.=1006 nm and X.=1098 nm.

3a. The Determination of Leached Dye Concentrations (Standard Protocol)

Dried particles (50 mg) were added to 3 mL of 1% sodium dodecyl sulfate to form a dispersion. The dispersion was sonicated for approximately 1 hour. The dispersion was centrifuged, and the supernatant component was withdrawn and filtered through a 0.2 p.m syringe filter. The absorbance spectrum of the filtrate was measured from 400-1300 nm using Shimadzu UV-3600 UV/VIS/NIR Spectrophotometer in a 1 cm cell.

The amount of the dye leached is calculated as in 2b above by applying Beer's law (Eq. 1) to give leachate concentrations. Leaching reduction is defined as the percentage of dye leached from coated particles when compared to uncoated particles of the same structure.

### Example 4

### Cellular Cytotoxicity Assay

4a. Cytotoxicity of Particle Components

Dye components were each dissolved in ethanol (molecular grade ethanol from Fisher Scientific) to produce a stock solution at 1 mM (B141, M071, and Epolight™ 1117) or 100 μM (C161). For each stock solution, additional dilutions were made at 2×, 4×, 8×, 16×, 32×, 64×, and 128×, and each concentration was tested for cytotoxicity against NIH 3T3 murine fibroblasts in a cytotoxicity test. NIH-3T3 cells were plated in a 96-well culture plate at a density of 10,000 cells per well and allowed to adhere to the surface overnight. Different concentrations of the dye solution were added to the NIH 3T3 cells and incubated for 24 hours at 37° C., in a 5% CO₂ incubator. Controls for the cytotoxicity experiment included “live” and “dead” (cells were killed due to osmotic pressure by adding D.I. water). “Live” cells had nothing except cell culture media containing 10% FBS added to them and were used to obtain the 100% viability data. The “dead” control was used to obtain the 0% viability data point. After 24 hours, cells were washed twice with 1× PBS containing calcium and magnesium and 100 μL of media was added at the end. To a final volume of 100 μL of media in the wells, 20 μL of PMS activated MTS reagent was added and incubated for 90 minutes. At the end of the 90 minutes, absorbance was measured at 490 nm using a plate reader (Spectramax M2e, Molecular Devices). Viability of cells was calculated using the absorbance measured for the “live” (100%) and “dead” (0%) controls and the results of % viability estimated from the absorbance for the different concentrations of the dyes were plotted in MS Excel using linear regression curve fitting algorithm to obtain the IC₃₀. All the samples were tested in triplicate and results were averaged over the three repeats.

Cytotoxicity of particle components, as described by the IC₃₀ concentration, is detailed in Table 4 below. The IR absorbing agent (the material) is cytotoxic at concentrations greater than about 41 μM. The dyes (the colorants) are cytotoxic at concentrations above about 61 μM for the Black and Magenta dyes, and above about 14 μM for the Cyan dye. It should be noted that the cytotoxicity of combinations of dyes can be unacceptable even if the leachate concentrations of all the components fall below their individual IC₃₀ values.

TABLE 4
Cellular cytotoxicity of particle components

| | | | IC₃₀ |
|---|---|---|---|
| Entry | Test | Component | (70% viability) |
| 1 | Black dye | B141 | 61.22 μM^{a} |
| 2 | Magenta dye | M071 | 63.23 μM^{a} |
| 3 | Cyan dye | C161 | 14.65 μM^{a} |
| 4 | Infrared dye | IR 1117 | 41.38 μM^{a} |

^{a}dye solution in ethanol

4b. Extractable Cytotoxicity Test (ECT)

100 mg of dry particles were weighed out and then suspended in 1 mL of cell culture media Dulbecco's Modified Eagle's medium (DMEM) containing 10% fetal bovine serum (FBS) and vortexed five times to ensure thorough mixing. This suspension was incubated at 37° C. for 24 hrs. After the incubation period, the suspension was centrifuged at 10,000 G for 10 minutes and the supernatant was collected. The supernatant solution was filtered through a 0.2 micron syringe filter and was used for cytotoxicity evaluation as the “neat” or 1X sample. This 1X neat extract was serially diluted with media containing 10% FBS for cytotoxicity testing. The following serial dilutions were made using the neat extract and the DMEM supplemented with 10%FBS: 2X (2-fold dilution), 4X (4-fold dilution), 8X (8-fold dilution), 16X (16-fold dilution) and 32X (32-fold dilution).

Inhibitory Concentration for 30% cell killing (IC₃₀) of the particle extract on NIH-3T3 cells (obtained from ATCC) was determined by performing an MTS assay, a standard colorimetric method to measure the cell viability following incubation with different dilutions of the 1X extract obtained above. NIH-3T3 cells were plated in a 96-well culture plate at a density of 10,000 cells per well and allowed to adhere to the surface overnight. Extract concentrations ranging from 1X to 32X were added and incubated for 24 hours at 37° C., in a 5% CO2 incubator. Controls for the cytotoxicity experiment included “live” and “dead” (cells were killed due to osmotic pressure by adding D.I. water). “Live” cells had nothing except cell culture media containing 10% FBS added to them and were used to obtain the 100% viability data. The “dead” control was used to obtain the 0% viability data point. After 24 hours, to a final volume of 100 μL of media in the cells, 20 μL of PMS activated MTS reagent was added and incubated for 90 minutes. The absorbance was measured at 490 nm using a plate reader (Spectramax M2e, Molecular Devices). Viability of cells was calculated using the absorbance measured at 1× dilution of the extract and the results of absorbance for serial dilutions 1× to 32× of the extract were plotted in MS Excel using linear regression curve fitting algorithm to obtain the IC₃₀. All the samples were tested in triplicate and results were averaged over the three repeats. A particle that results in a 70% cell viability in the cytotoxicity test is considered passing the Extractable Cytotoxicity Test.

In an embodiment, a particle example that results in 70% cell viability (or higher) in the Extractable Cytotoxicity Test at the original extract concentration (1X) is considered passing the ECT criteria. In an embodiment, a particle example demonstrating results in 70% cell viability (or higher) in the cytotoxicity test at 10-fold dilution (0.1X) is considered passing the ECT criteria. In an embodiment, a particle example showing results in 70% cell viability (or higher) in the cytotoxicity test at 100-fold dilution (0.01X) is considered passing the ECT criteria. In some instances, if the neat or dilution concentration of the colorant and the material in the leachate is independently less than IC₁₀, IC₃₀, IC₄₀, IC₅₀, IC₆₀, IC₇₀, IC₈₀, or IC₉₀, the particle passes the Extractable Cytotoxicity Test.

4c. Cytotoxicity of Particle Structures.

Cytotoxicity of particles was determined using the ECT described above. The polymer carrier is known to be a biocompatible material and was only tested at a concentration comparable to what would be used in practice, and as shown in Table 5, it is not cytotoxic (greater than 70% cell viability) at this concentration. The effect of the use of a protective shell on particle cytotoxicity are also described Table 5 below. The use of a VTMS shell significantly reduced the cytotoxicity when compared to particles without such shells. From the data in Table 5, it is shown that the presence of the VTMS shell is critical to meeting the requirements of the Extractable Cytotoxicity Test (ECT).

TABLE 5
Cellular cytotoxicity of PB1 particle structures

| | | Polymer^{a}/dye | Particle | | Cytotoxicity |
|---|---|---|---|---|---|
| entry | Particle Color | ratio | size (μm) | Shell | (% viability)^{b} |
| 1 | Empty B805^{a} | — | 3 | none | 92.4 |
| 2 | PB1 | 3:1 | 0.5 | none | 51.5 |
| 3 | PB1 | 3:1 | 3 | none | 58.5 |
| 4 | PB1 | 3:1 | 3 | 25 wt. % | 79.7 |
| | | | | VTMS | |

^{a}B805 polymer, MMA/BMA copolymer
^{b}in vitro cellular viability at 1X strength of the dye extract (70% viability is considered passing)

### Example 5

### Effects of Shell Matrix Material on the Leaching of Payloads from the Particle

5a. Impact of VTMS Shell on Leaching

Particles were prepared with 3 μm cores containing 3:1 weight ratio of polymer to the PB1 dye composition. Portions of these particles were coated with several different shell thicknesses as described in Example lb above and tested for effectiveness in preventing dye leaching following the surfactant-based extractable test described in section Example 3b. Tables 6A, 6B, and 6C summarize the leaching from 3 μm PB1 particles to which a 40% shell (0.66:1 VTMS:uncoated particles), a 25% shell (0.33:1 VTMS: uncoated particles), and a 9.1% shell (0.1:1 VTMS: uncoated particles) were applied, respectively.

The results in Tables 6A.-6C. showed that increasing the shell thickness generally can reduce the leaching of the payloads. For example, a particle having a 25% VTMS shell exhibited better results in reducing the leakage of dye as compared with a particle having a 9.1% VTMS (Tables 6B. and 6C.). However, the further increasing the concentration of VTMS starting material in solution from 25 wt. % to 40 wt. % of the weight of the particle core did not yield reducing dye leaching when compared the particle having a 25% VTMS shell (Tables 6A. and 6B.).

TABLE 6A
Dye leaching from 3 μm 3:1 PB1 particles (FIG. 3)
3 μm 3:1 PB1 Particles

| | | | | | IR1117 |
|---|---|---|---|---|---|
| | | B141 | M071 | C161 | (1064 |
| | | (606 nm) | (558 nm) | (680 nm) | nm) |
| Uncoated | Leachate | 54.2 | 17.1 | 10.8 | 18.6 |
| particle | concentration | | | | |
| | (μM)^{a} | | | | |
| with a 40% | Leachate | 6.8 | 1.1 | 0.6 | 0.1 |
| VTMS shell | concentration | | | | |
| | (μM)^{a} | | | | |
| | Reduced | 12.5% | 6.3% | 5.5% | 0.6% |
| | leaching to | | | | |
| | Reduction | 8.0 | 15.8 | 18.0 | 176.6 |
| | factor^{b} | | | | |

^{a}The concentrations of the leached dyes from the uncoated particles were calculated using Eqn 1 in Example 2b above.
^{b}The amount of dye leaching reduced was defined in Example 3b above.

TABLE 6B
Dye leaching from 3 μm 3:1 PB1 particles
3 μm 3:1 PB1 Particles

| | | | | | IR1117 |
|---|---|---|---|---|---|
| | | B141 | M071 | C161 | (1064 |
| | | (606 nm) | (558 nm) | (680 nm) | nm) |
| Uncoated | Leachate | 59.9 | 18.7 | 11.8 | 19.0 |
| particle | concentration | | | | |
| | (μM)^{a} | | | | |
| with a 25% | Leachate | 6.5 | 1.1 | 0.6 | 0.1 |
| VTMS shell | concentration | | | | |
| | (μM)^{a} | | | | |
| | Reduced | 10.9% | 5.6% | 5.1% | 0.3% |
| | leaching to | | | | |
| | Reduction | 9.2 | 17.8 | 19.7 | 361.4 |
| | factor^{b} | | | | |

^{a}The concentrations of the leached dyes from the uncoated particles were calculated using Eqn 1 in Example 2b above.
^{b}The amount of dye leaching reduced was defined in Example 3b above.

TABLE 6C
Dye leaching from 3 μm 3:1 PB1 particles
3 μm 3:1 PB1 Particles

| | | | | | IR1117 |
|---|---|---|---|---|---|
| | | B141 | M071 | C161 | (1064 |
| | | (606 nm) | (558 nm) | (680 nm) | nm) |
| Uncoated | Leachate | 60.1 | 18.0 | 13.2 | 20.7 |
| Particle | concentration | | | | |
| | (μM)^{a} | | | | |
| with a 9.1% | Leachate | 11.8 | 2.2 | 1.4 | 0.3 |
| VTMS shell | concentration | | | | |
| | (μM)^{a} | | | | |
| | Reduced | 19.7% | 12.0% | 10.6% | 1.3% |
| | leaching to | | | | |
| | Reduction | 5.1 | 8.3 | 9.4 | 75.5 |
| | factor^{b} | | | | |

^{a}The concentrations of the leached dyes from the uncoated particles were calculated using Eqn 1 in Example 2b above.
^{b}The amount of dye leaching reduced was defined in Example 3b above.

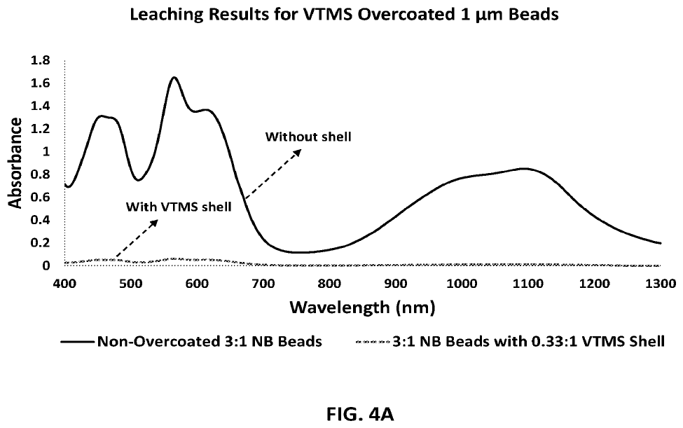

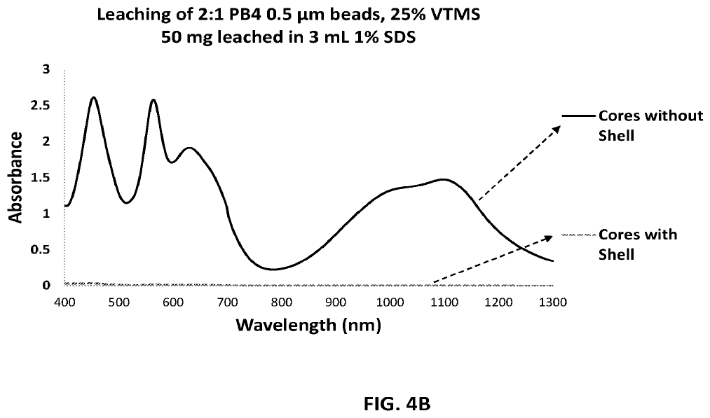

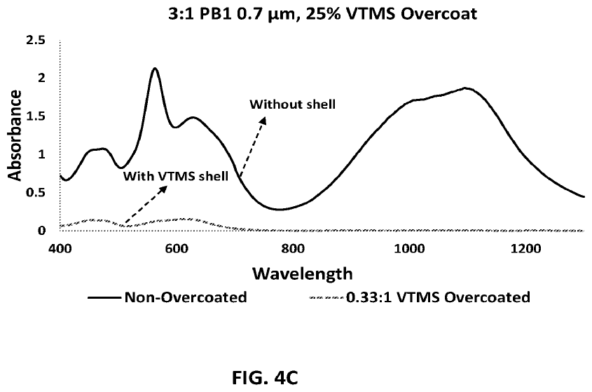

The 25% VTMS shell proved effective on multiple different iterations of particles, as seen in Tables 7A, 7B, and 7C, significantly reducing the leaching of dyes from particles with shells as compared to leaching from the uncoated particles. The absorbance spectra for leachates are illustrated in FIGS. 4A-4C. The concentration of the leached dyes and the dye leaching reduction for various particles are summarized in Tables 7A-7C.

TABLE 7A
Dye leaching from 1 μm 3:1 NB particles (FIG. 4A)
1 μm 3:1 NB Particle

| | | B141 | M071 | IR1117 |
|---|---|---|---|---|
| | | (606 nm) | (558 nm) | (1064 nm) |
| Uncoated | Leachate | 73.4 | 17.8 | 8.7 |
| particle | concentration | | | |
| | (μM)^{a} | | | |
| with a 25% | Leachate | 2.8 | 0.6 | 0.1 |
| VTMS shell | concentration | | | |
| | (μM)^{a} | | | |
| | Leaching | 3.8% | 3.4% | 1.2% |
| | reduced to | | | |
| | Reduction | 26.2 | 29.7 | 87.0 |
| | factor^{b} | | | |

^{a}The concentrations of the leached dyes from the uncoated particles were calculated using Eqn 1 in Example 2b above.
^{b}The amount of dye leaching reduced was defined in Example 3b above.

TABLE 7B
Dye leaching from 0.5 μm 2:1 PB4 particles (FIG 4B)
0.5 μm 2:1 PB4 Particles

| | | B141 | M071 | C161 | Y161 | IR1117 |
|---|---|---|---|---|---|---|
| | | (606 nm) | (558 nm) | (680 nm) | (454 nm) | (1064 nm) |
| Uncoated | Leachate | 96.1 | 28.0 | 20.7 | 87.1 | 14.9 |
| particle | concentration | | | | | |
| | (μM)^{a} | | | | | |
| with a 25% | Leachate | 0.8 | 0.2 | 0.2 | 1.2 | 0.0 |
| VTMS shell | concentration | | | | | |
| | (μM)^{a} | | | | | |
| | Leaching reduced to | 0.8% | 0.7% | 1.0% | 1.4% | 0.3% |
| | Reduction Factor^{b} | 120.1 | 140.0 | 103.5 | 72.6 | 333.3 |

^{a}The concentrations of the leached dyes from the uncoated particles were calculated using Eqn 1 in Example 2b above.
^{b}The amount of dye leaching reduced was defined in Example 3b above.

TABLE 7C
Dye leaching from 0.7 μm 3:1 PB1 particles (FIG. 4C)
0.7 μm 3:1 PB1 Particles

| | | | | | IR1117 |
|---|---|---|---|---|---|
| | | B141 | M071 | C161 | (1064 |
| | | (606 nm) | (560 nm) | (680 nm) | nm) |
| Uncoated | Leachate | 75.6 | 23.4 | 16.0 | 18.9 |
| particle | concentration | | | | |
| | (μM)^{a} | | | | |
| with a 25% | Leachate | 8.1 | 1.3 | 0.8 | 0.1 |
| VTMS shell | concentration | | | | |
| | (μM)^{a} | | | | |
| | Leaching | 10.7% | 5.6% | 5.0% | 0.5 |
| | reduced to | | | | |
| | Reduction | 9.3 | 18.0 | 20.0 | 189.0 |
| | factor^{b} | | | | |

^{a}The concentrations of the leached dyes from the uncoated particles were calculated using Eqn 1 in Example 2b above.
^{b}The amount of dye leaching reduced was defined in Example 3b above

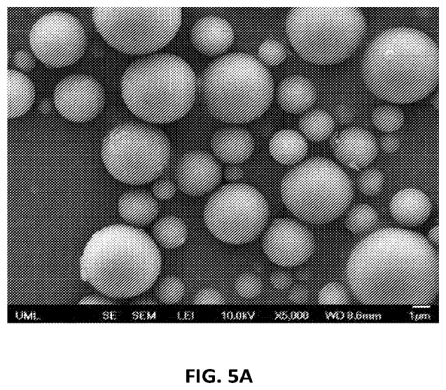

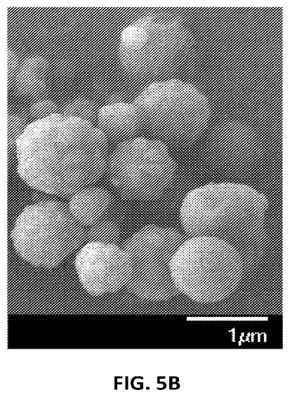

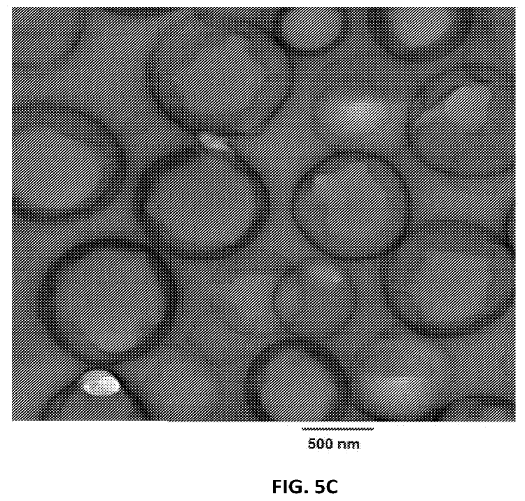

The scanning electron microscope (SEM) image for 1 μm uncoated particles and for 1 μm 3:1 neutral black particles having a 25% VTMS shell is shown in FIG. 5A and 5B, respectively. The presence of the organosilicate shell is evident from the change in surface morphology from a smooth surface on uncoated particles to the irregular, rough surface on the particles with shells.

FIG. 5C illustrates the transmission electron microscope (TEM) images for cross-sectioned 0.7 μm 3:1 process black 1 particles having a 25% VTMS shell. The presence of a thin, uniform shell is evident from the dark, circular ring around each particle.

5b. Effects of TEOS as Shell Material Upon the Leachability of the Payloads

The effects of shell made from tetraethoxysilane (TEOS) upon leaching of the payloads have been studied using the particles fabricated under the same conditions of separate, acidic hydrolysis followed by condensation at pH 10 as those used for the VTMS shell construction. The cross-linking reaction was checked after 2, 4, and 26 hours. Reductions in leaching for each of these was very low, with the 26-hour reaction only yielding a 20% reduction in leachate. While IR absorbing agent leaching had been reduced more than that of the visible dyes, measurement of the dye content left in the particles indicated a loss of >40% of the IR absorbing agent. The absorbance spectra for the leached payloads from the particles having TEOS as shell are summarized in Table 8 below.

TABLE 8
Dye leaching from 0.7 μm, 3:1
Process Black 1 (PB1) particles^{a} (FIG. 6).

| | | | | | IR1117 |
|---|---|---|---|---|---|
| | | B141 | M071 | C161 | (1064 |
| | | (606 nm) | (560 nm) | (680 nm) | nm) |
| Uncoated | Leachate | 87.5 | 23.9 | 17.2 | 19.0 |
| | concentration | | | | |
| | (μM)^{b} | | | | |
| With a | Leachate | 70.6 | 19.1 | 13.6 | 8.8 |
| 25 wt. % | concentration | | | | |
| TEOS shell, | (μM)^{b} | | | | |
| 26 Hours | Leaching | 80.7% | 79.9% | 79.01 | 46.3% |
| | reduced to | | | | |
| | Reduction | 1.2 | 1.3 | 1.3 | 2.2 |
| | Factor^{c} | | | | |

^{a}Particles contained no Cyanox ® 1790
^{b}The concentrations of the leached dyes from the uncoated particles were calculated using Eqn 1 in Example 2b above.
^{c}The calculation for the amount of dye leached reduced is defined in Example 3b above

The results in Table 8 and in FIG. 6 showed that the shell made from 25 wt. % TEOS alone does not provide sufficient reduction in leaching of the payloads from the particle as compared with the shell made from VTMS even under the conditions that the thickness of shell from TEOS is greater than that of the shell from VTMS.

5c. Effects of Shell Material Combinations on the Leaching of the Payloads

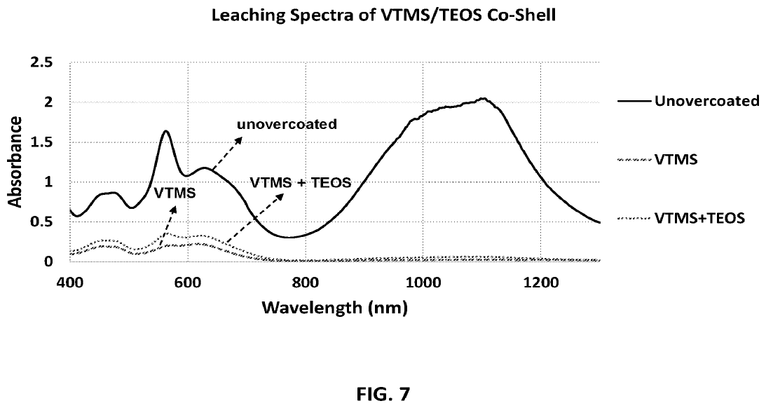

A shell comprising both VTMS and TEOS was constructed on uncoated particles containing the PB1 dye composition. VTMS was added first, at ¼ the normal level (weight ratio of 1:1 VTMS:uncoated particle in the reaction mixture, equivalent to 9.1% VTMS) and condensed at pH 8 for 2 hours, followed by the addition of TEOS at a 3× the molar amount of VTMS (weight ratio of 0.1:0.42:1 VTMS:TEOS:uncoated particles), and condensed at pH 8 for additional 24 hours. With this procedure, it was expected that the TEOS would condense onto an initially formed VTMS shell to produce a finished coating with greater cross-link density. The leaching test results in Table 9 below and in FIG. 7 showed that the TEOS/VTMS shell performed worse than the shell produced with only VTMS.

TABLE 9
Dye leaching from 0.9 μm, 3:1 PBI particles^{a} (FIG. 7).

| | | B141 | M071 | C161 | IR1117 |
|---|---|---|---|---|---|
| | | (606 nm) | (560 nm) | (680 nm) | (1064 nm) |
| Uncoated | Leachate | 60.1 | 18.0 | 13.2 | 20.7 |
| | concentration | | | | |
| | (μM)^{b} | | | | |
| With a | Leachate | 11.8 | 2.2 | 1.4 | 0.3 |
| 25% | concentration | | | | |
| VTMS | (μM)^{b} | | | | |
| shell | Leaching | 19.6% | 12.2% | 10.6% | 1.5% |
| | reduced to | | | | |
| | Reduction | 5.1 | 8.2 | 9.4 | 69.0 |
| | Factor | | | | |
| With a | Leachate | 17.2 | 3.8 | 2.5 | 0.7 |
| VTMS + | concentration | | | | |
| TEOS | (μM)^{b} | | | | |
| (1:3) | Leaching | 28.6% | 21.1% | 19.3% | 3.2% |
| shell | reduced to | | | | |
| | Reduction | 3.5 | 4.7 | 5.3 | 29.6 |
| | Factor | | | | |

^{a}Particles contained no Cyanox ® 1790.
^{b}The concentrations of the leached dyes from the uncoated particles were calculated using Eqn 1 in Example 2b above.
^{c}The calculation for the amount of dye leached reduced is defined in Example 3b above.

Effects of shells made from various different silane reagents on the payloads have been studied. The dye particles were coated with shells made from various different types of trimethoxysilane derivative including n-octyltriethoxysilane, 2-[methoxy(polyethyleneoxy)6-9 propyl]trimethoxysilane, and 3-(trimethoxysilyl)propyl methacrylate. The leaching test protocol as set forth above was performed on each of the particles coated with different trimethoxysilane derivatives. None of the shells gave improved leaching over the shell made from TEOS or VTMS alone.

### Example 6

### Efficacy Determination Protocol

An Efficacy Determination Protocol is used to evaluate the effect of biological chemicals including bodily fluid on the colorant and/or the material that are encapsulated inside the particle. Briefly, a known quantity of the particles containing the colorant is incubated with 1mL of complete cell culture media (for example macrophage or neutrophil cell growth media) containing 10% fetal bovine serum at 37° C. As a negative control, the same quantity of particles containing the colorant is suspended in 1mL of distilled water and incubated at 37° C. At different time intervals (for example: 24 h, 48 h, 72 h, 120 h) following incubation, for both the test and control, a small portion of the sample is removed and diluted with distilled water. If the colorant absorbs UV-VIS-IR, then the UV-VIS-IR absorbance spectrum of each solution is measured using a UV-VIS-IR spectrophotometer. Degradation of the chemical agents by the cell culture medium is determined by comparing the peak absorption in the spectrum of the test sample to the absorption of the control sample at the same spectral peak, and degradation is generally reported as the percentage in the reduction in the peak absorbance. If the colorant does not absorb UV-VIS-IR, other analytical tools, like NMR, HPLC, LCMS etc., would be used to quantify the concentration of the colorant in the test and control. The particles can be designed to ensure that no more than 90% degradation is observed at 24 h following incubation with relevant cell culture media.

In an embodiment, the degree of degradation for the dye encapsulated within the particle can be determined using the dye loading determination protocol set forth in Example 3a. above. The degradation of non-encapsulated colorant can also be compared to that of the encapsulated colorant to evaluate the effect of encapsulation in particles. Depending on the application, different biological agents can be added to the cell culture media to simulate conditions that occur in vivo. This protocol in conjunction with the Extractable Cytotoxicity Test will provide feedback (feedback loop protocol) to optimize the particle structure such that the colorant and/or the material can be protected from the degradation by body chemicals. The Extractable Cytotoxicity Test was conducted according to the protocols set forth above.

### Example 6a

### The Stability of IR absorbing agent Compound in Neutrophil and Macrophage Medium

An IR absorbing agent stock solution was prepared by dissolving 10.4 mg of IR absorbing agent (Epolight™ 1117) in 250 mL of methanol solvent.

Control solutions for tests of the IR absorbing agent stability were prepared by 1:1 dilution of the IR absorbing agent stock solution with 1.5 mL of distilled water. Test samples for stability in biological media were prepared by dilution of 1.5 mL of IR absorbing agent stock solution with 1.5 mL of media (neutrophil or macrophage media). All samples were vortexed at room temperature and sampled periodically over 20 minutes. Samples were analyzed by absorbance measured with a Shimadzu UV-3600 UV/VIS/NIR-in the IR spectrophotometer band. The testing results are as illustrated in FIG. 8 and FIG. 9.

The results in FIG. 8 and FIG. 9 showed that direct contact of IR absorbing agent with both neutrophil and macrophage media caused rapid degradation of IR absorbing agent. The results showed that body chemicals in the neutrophil and macrophage media can cause degradation of unprotected IR absorbing agent.

### Example 6b

### The Stability of Dye Encapsulated in Polymeric Particles in Neutrophil and Macrophage Medium

Aliquots of 100 mg of particles were placed in each of 900 μl volumes of distilled water, phosphate buffer solution (PBS), complete macrophage media, and complete neutrophil media. Each sample was vortexed to suspend the particles, and all samples were incubated at 40° C. Samples were analyzed at 0 hours, 22 hours, 42 hours, and 107 hours of incubation by withdrawing 20 μ1 and diluting into 3 ml of distilled water. Absorbance spectra were captured on a Shimadzu UV-3600 UV/VIS/NIR spectrophotometer over the range 320-1300 nm. Spectra were normalized to the peak of the M071 dye, and loss of IR absorbing agent was determined by changes in absorption at 1064 nm.

Results of the Efficacy Determination Protocol (EDP) are shown in Table 10. Treatment with water resulted in no loss of IR absorbing agent absorption in either the uncoated or coated particles, with any changes over time being representative of test variability. Treatment of particles with phosphate buffer resulted in small loss in the uncoated particles, but no change in the particles with the VTMS shell. Both macrophage and neutrophil media led to a loss of about 15% over 107 hours in uncoated particles. Only a small loss of about 5% was observed in the case of the particles with VTMS shells. The presence of the VTMS shell significantly improved the retention of IR absorbing agent in the coated particles.

TABLE 10
EDP: Retention of IR absorbing agent in 2 μm, 2:1
PB4 particles treated with biological media.

| Particle | Media | 0 hrs | 22 hrs | 42 hrs | 107 hrs |
|---|---|---|---|---|---|
| Uncoated | Distilled H₂O | 100.0% | 100.6% | 100.7% | 100.7% |
| particles | PBS | 100.0% | 98.2% | 98.1% | 96.4% |
| | Macrophage | 100.0% | 94.0% | 91.3% | 86.1% |
| | media | | | | |
| | Neutrophil | 100.0% | 93.3% | 91.0% | 85.2% |
| | media | | | | |
| Particles with | Distilled H₂O | 100.0% | 101.9% | 101.4% | 103.2% |
| 25% VTMS | PBS | 100.0% | 100.5% | 101.7% | 101.8% |
| shell | Macrophage | 100.0% | 98.1% | 97.3% | 95.3% |
| | media | | | | |
| | Neutrophil | 100.0% | 98.1% | 97.6% | 94.6% |
| | media | | | | |

From the results of the Efficacy Determination Protocol (EDP) on dyed particles, neither the uncoated and coated particles show degradation that would be expected to significantly reduce performance of the particles in the tattoo application. While the presence of shells improves the EDP performance, the shells are critical to meeting the requirements of the Extractable Cytotoxicity Test (ECT).

### Example 7

### Material Process Stability Test

Particle heaters are dispersed in a 2% solution of gelatin in warm water. The suspension is vortexed and transferred to 50 mm plastic culture dishes and allowed to gel, producing a greenish gel. The optical density is measured by reflectance spectroscopy to provide a baseline absorbance.

Areas on the culture dishes are irradiated over a range of pulse widths and fluences that span the conditions expected for use. Generally, pulse widths range from about 100 us to about 1 second, with fluences that range from about 0.1 J/cm² to about 60 J/cm². The absorbance is measured for each exposure condition and compared to the baseline absorbance. The preservation greater than 50% absorbance of the material after subject to such process conditions is considered to pass the Material Process Stability Test.

### Example 8

### Laser Triggered Changing of Color of Tattoo Particles

A series of experiments was performed to demonstrate the efficacy of color change in particles containing colorant and material. Generally, particles were irradiated in gelatin followed by spectroscopic analysis of the components, including the IR1117 material. Since the in vitro experiment was not designed to assure complete irradiation of all particles, the degree of color removal cannot be taken as an indicator of expected color change performance in an in vivo application.

Procedure: A solution of gelatin was prepared by adding Knox gelatin (1.0 g) to cold water (12.5 g) in a 100 mL glass jar equipped with a magnetic stir bar. The gelatin was stirred 15-30 minutes and then hot water was added (70° C.) until the total weight was 50.0 g. This gelatin was then used in 2.0 gm aliquots. Colorant loaded PMMA-BMA B-805 copolymer particles with 25% VTMS shells as prepared in Example lb above (20-30 mg) were then added to the gelatin solution (2.0 g) and vortexed in a 4 dram vial. The suspension of the tattoo particles in gelatin was then sonicated for 15-30 minutes before transferring to a 5 cm plastic culture dish. The gelatin suspension was spread evenly and allowed to set. It was then covered and stored at 6° C. until used.

Laser exposure was accomplished as follows: The cover for the culture dish was removed and a 5 cm clear plastic cover was cut and fit over the gelatin to prevent splatter. The top surface was then completely irradiated at 1064 nm with a 5 mm spot using fluences ranging from 2.46 J/cm² to 5.09 J/cm² using a Nd-YAG Q-switched Lutronic laser (Lutronic Spectra™ VRM II Laser with four distinct Q-switched mode wavelengths: 1064 nm, 532 nm, 585 nm, 650 nm, nano second pulse width, and spectra peak energy: 60 MW, 120 MW and 240 MW).

After irradiating the top surface, the culture dish was covered with its lid, turned over and irradiated from the opposite side to reach any unexposed particles visible only from the bottom side.

Following the complete irradiation, the plastic cover was removed and any adhered gelatin was transferred to a 10 ml centrifuge tube. The gelatin from the culture dish was also removed and transferred to the centrifuge tube with the aid of about 5 ml of water. The culture tube was rinsed with water and any suspended gelatin transferred by pipette to the centrifuge tube. The material in the tube was sonicated until the gelatin was redissolved (20-30 minutes, about 40° C.).

The sample was centrifuged for 20 minutes and the supernatant was removed. The recovered particles were slurried again with water, centrifuged, and the wash water removed. The resultant particles were dried in vacuum at room temperature and analyzed spectroscopically for the presence of dyes as in 2b above.

The standard fluence for removal of tattoo color in designs using these particles is 3.51 J/cm². 5.09 J/cm² is the maximum fluence on the Lutronic laser using a 5 mm spot. The laser triggered color changing tests were performed on 2 μm 5:1 Y197 (12.5% Y197:6.25% IR 1117), 2 μm 7:1 M071 (6.25% M071, 8.0 wt. % IR 1117), 2 μm 5:1 PBS (2.56 wt. % B141, 0.77 wt. % C161, 0.39 wt. % M071, 1.28 wt. % Y184, 8.0 wt. % IR1117), 2 μm 5:1 Y184 (12.5 wt. % Y184, 8.0 wt. % IR 1117) particles in the entries 21-24 of Table 3 above.(the numeric ratio is weight ratio of PMMA-BMA B-805 copolymer to colorant in the particle)

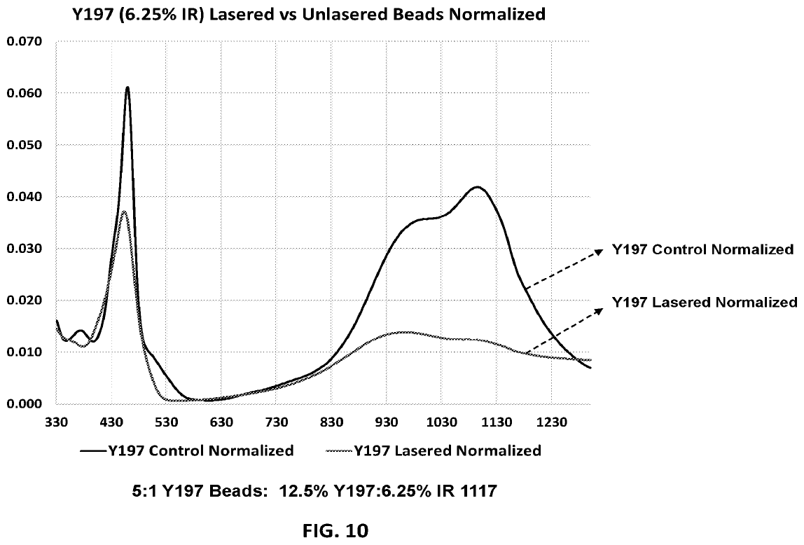

The laser triggered color change results for Y197 particles are summarized in FIG. 10 and Table 11 below.

TABLE 11
Spectroscopic change of particles comprising
Y197 and IR absorbing agent at 3.51 J/cm²

| Fluence | Abs Y197 | % | Abs IR | % |
|---|---|---|---|---|
| (J/cm²) | (458 nm) | Reduction | (1064 nm) | Reduction |
| 0 | 0.061 | 0 | 0.039 | 0 |
| 3.51 | 0.036 | 41 | 0.013 | 67 |

It was observed that the IR absorbing agent was substantially reduced in Y197 particles indicating a substantial absorption of IR radiation and subsequent heat generation and loss of IR absorbing agent (69%). Y197 was found to be reduced modestly in density (41%).

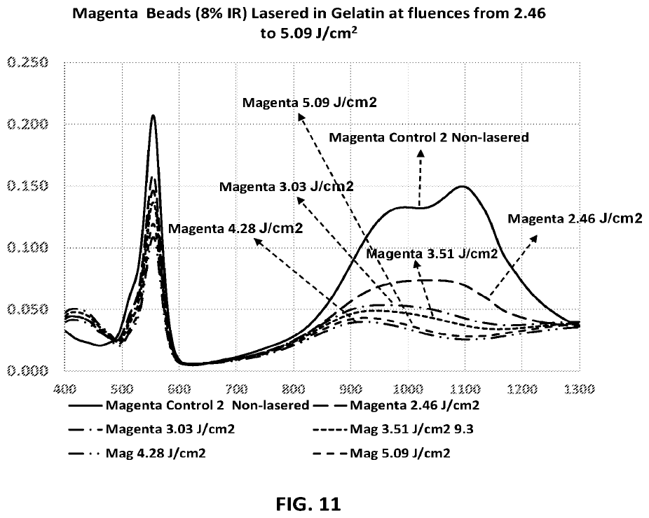

The laser triggered color change results for M701 particles at different fluences are summarized in FIG. 11 and Table 12 below.

TABLE 12
Spectroscopic change of particles comprising M071 and
IR absorbing agent at different fluences

| Fluence | Abs M071 | % | Abs IR | % |
|---|---|---|---|---|
| (J/cm²) | (458 nm) | Reduction | (1064 nm) | Reduction |
| 0 | 0.207 | 0 | 0.145 | 0 |
| 2.46 | 0.159 | 23 | 0.073 | 50 |
| 3.03 | 0.146 | 30 | 0.048 | 67 |
| 3.51 | 0.137 | 34 | 0.041 | 72 |
| 4.28 | 0.109 | 47 | 0.027 | 81 |
| 5.09 | 0.119 | 43 | 0.030 | 79 |

It was observed that the reduction of the IR absorbing agent in M071 particles was 80%, contrasting with a reduction of about 50% for the magenta dye. Furthermore, the dye reduction in M071 particles appeared to level off at 4.28 J/cm².

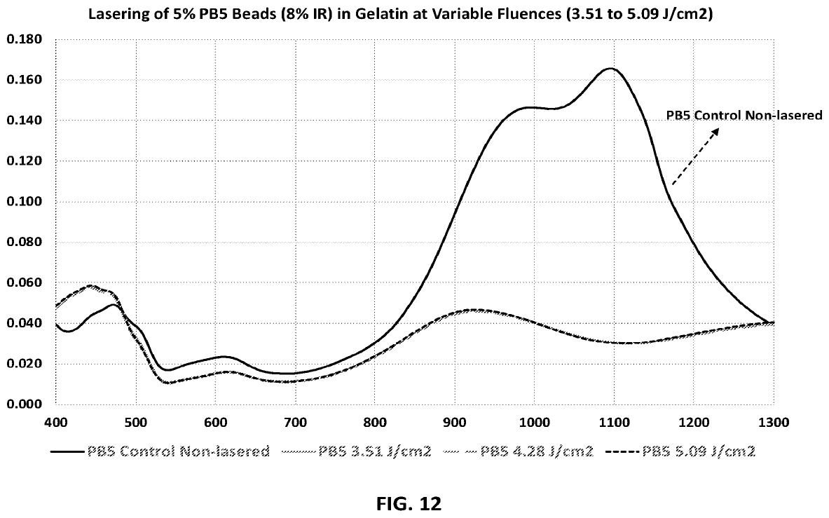

The laser triggered color change results for 5% PB5 particles at different fluences are summarized in FIG. 12 and Table 13 below.

TABLE 13
Spectroscopic change of particles comprising 5%
PB5 and IR absorbing agent at different fluences

| Fluence | Abs M071 | % | Abs IR | % |
|---|---|---|---|---|
| (J/cm²) | (458 nm) | Reduction | (1064 nm) | Reduction |
| 0 | 0.023 | 0 | 0.156 | 0 |
| 3.51 | 0.016 | 30 | 0.033 | 79 |
| 4.28 | 0.016 | 30 | 0.032 | 80 |
| 5.09 | 0.016 | 30 | 0.032 | 80 |

It was observed that the reduction of 5% PB5 in the particles stopped at about 30%. leveling off at a fluence of 3.51 J/cm². No additional heat was generated from the higher fluence which suggests that the IR absorbing agent absorbance was saturated at 3.51 J/cm².

### Example 9

### Application and Color Removal of Tattoo Prepared with 5% PB5 Particles

PMMA particles comprising 5% PBS and IR absorbing agent and coated with 25% VTMS shells were prepared as in Example lb. These particles were suspended in a liquid carrier and applied to skin using common tattoo equipment and techniques. Following a three-week healing process, the tattooed skin was measured for color density using an X-Rite Ci64UV sphere colorimeter. At 257 days following application of the tattoo, the tattoo was exposed to a single pulse of 1064 nm infrared light at 3.51 J/cm² within a 5 mm spot size with a pulse duration of 15 ns using a Lutronics Spectra VRM II nanosecond laser, leading to reduction in color of the tattoo.

The initial skin tone before application of the tattoo was described by having a black density of 0.53. The density of the tattoo before irradiation was 0.91 following healing of the tattoo application. After laser irradiation, the black density in the irradiated area was 0.56.

Spectral readings demonstrate a reduction of the color of the tattoo following irradiation, resulting in a skin tone the same color as the surrounding skin that had not been affected by the tattoo.

We claim:

1. A particle comprising: a core comprising a carrier, a material, and a colorant; a shell enclosing the core; wherein the color of the colorant changes when the material absorbs radiation at infrared wavelengths; wherein the colorant and the material in the particle exhibit stability such that the particle is considered passing the Efficacy Determination Protocol; and wherein the particle structure is constructed such that it passes the Extractable Cytotoxicity Test.

2. The particle of claim 1, wherein the carrier comprises a polymer or copolymer of methylmethacrylate.

3. The particle of claim 1, wherein the shell is a cross-linked polymer.

4. The particle of claim 3, wherein the shell comprises a silicate polymer derived from vinyl trimethoxysilane.

5. The particle of any one of claims 1-4, wherein the infrared wavelengths of the radiation are in the range of 700 to 1500 nm.

6. The particle of any one of claims 1-4, wherein the infrared wavelength of the radiation is at 1064 nm.

7. The particle of any one of claims 1-4, wherein the material for absorbing the radiation at infrared wavelengths is a tetrakis aminium dye.

8. The particle of any one of claims 1-6, wherein the material for absorbing the radiation at infrared wavelengths is Epolight IR 1117.

9. The particle of any one of claims 1-6, wherein the material for absorbing the radiation at infrared wavelengths is a zinc iron phosphate pigment.

10. The particle of claims 1, wherein the colorant comprises a chromophore group and a thermally activatable fragmentation group.

11. The particle of any one of claims 1-10, wherein the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof.

12. The particle of any one of claims 10-11, wherein the thermally activatable fragmentation group produces a nucleophilic group upon activation.

13. The particle of any one of claims 10-11, wherein the thermally activatable fragmentation group comprises substituted and unsubstituted carbonates, carbamates, esters, lactams, lactones, amides, imides, oximes, sulfonates, or phosphonates.

14. A particle comprising: (a) a core comprising a carrier, a material, and a thermally-activatable colorant; and (b) a shell enclosing the core; wherein the shell comprises a cross-linked organo-silicate polymer derived from trialkoxysilane, or trihalorosilane; wherein the thermally activatable colorant is transformed to colorless when the material absorbs radiation at infrared wavelengths and converts the energy to heat; wherein the thermally activatable colorant and the material in the particle exhibit stability such that the particle is considered passing the Efficacy Determination Protocol; and wherein the particle structure is constructed such that it passes the Extractable Cytotoxicity Test.

15. The particle of claim 14, wherein the carrier comprises a polymer or copolymer of methylmethacrylate.

16. The particle of any one of claim 14-15, wherein the trialkoxysilane used for making the shell is selected from the group of C2-C7 alkyl-trialkoxysilane, C2-C7 alkenyl-trialkoxysilane, C2-C7 alkynyl-trialkoxysilane, aryl-trialkoxysilane, and combinations thereof.

17. The particle of any one of claim 14-15, wherein the trihalosilane used for making the shell is selected from the group of trichlorosilane, tribromosilane, triiodosilane, and combinations thereof.

18. The particle of any one of claim 14-17, wherein the cross-linked organo-silicate polymer is derived from vinyl-trimethoxysilane (VTMS).

19. The particle of any one of claims 14-18, wherein the thermally activatable colorant comprises a chromophore group and a thermally activatable fragmentation group.

20. The particle of claim 19, wherein the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof.

21. The particle of any one of claims 19-20, wherein the thermally activatable fragmentation group produces a nucleophilic group upon activation.

22. The particle of any one of claims 19-20, wherein the thermally activatable fragmentation group comprises substituted and unsubstituted carbonates, carbamates, esters, lactams, lactones, amides, imides, oximes, sulfonates, or phosphonates.

23. The particle of claims 14-22, wherein the material absorbs the infrared radiation having wavelengths in the range of 700 to 1500 nm.

24. The particle of claim 23, wherein the material absorbs the infrared radiation having wavelength at 1064 nm.

25. The particle of claim 23, wherein the material for absorbing infrared radiation is a tetrakis aminium dye.

26. The particle of claims 23-25, wherein the material for absorbing the radiation at infrared wavelengths is Epolight IR 1117.

27. A tattoo ink for the permanent removable tattoo comprising the tattoo particles of any one of claims 1-26 and a dermatologically acceptable liquid carrier.

28. The tattoo ink of claim 27, wherein the tattoo ink is in the form of an injectable suspension.

29. The tattoo ink of claim 27, wherein the dermatologically acceptable liquid carrier is selected from the group of purified water, witch hazel, Listerine® mouthwash, and buffer solution.

30. The tattoo ink of any one of claims 27-29, wherein the dermatologically acceptable liquid carrier comprises a buffer solution having pH ranging from about 6 to about 8.

31. The tattoo ink of claim 30, wherein the buffer solution is a hydrogen ion buffer selected from the buffers listed in Table A.

32. A method of making permanent removable tattoos in a subject comprising the step of injecting the tattoo ink of any one of claims 27-31 to an area of skin of the subject to form a permanent tattoo on the subject.

33. A method of remotely triggering color-change from tattoo particles comprising the step of administering a dose of laser to the permanent tattoos on the subject of the claim 32.

34. The method of claim 33, further comprises repeating the step of claim 33.

35. The method of claim 33, wherein the laser is a pulsed laser light.

36. The method of claim 33, wherein the laser pulse duration is in a range from milliseconds to nanoseconds, and the laser has an oscillation wavelength at 1064 nm.

37. The method of claim 35, wherein the laser emits light at 808 nm.

38. The method of claim 35, wherein the laser emits light at 805 nm.

39. A particle comprising a carrier, a material, and a colorant, wherein the colorant is transformed to colorless when the material absorbs radiation at infrared wavelengths; wherein the colorant and the material in the particle exhibit stability such that the particle is considered passing the Efficacy Determination Protocol; and wherein the particle structure is constructed such that it passes the Extractable Cytotoxicity Test.

40. The particle of claim 39, wherein the carrier comprises a polymer or copolymer of methylmethacrylate.

41. The particle of claims 39-40, wherein the infrared wavelengths of the radiation are in the range of 700 to 1500 nm.

42. The particle of any one of claims 40-41, wherein the infrared wavelength of the radiation is at 1064 nm.

43. The particle of any one of claims 40-42, wherein the material for absorbing the radiation at infrared wavelengths is a tetrakis aminium dye.

44. The particle of any one of claims 40-42, wherein the material for absorbing the radiation at infrared wavelengths is a zinc iron phosphate pigment.

45. The particle of any one of claims 39-44, wherein the colorant comprises a chromophore group and a thermally activatable fragmentation group.

46. The particle of claim 45, wherein the chromophore group is selected from a substituted or unsubstituted triarylmethane, a xanthene, a rhodamine, a fluoran, an azocarbocyanine, a benzilidine, a thiazine, an acridine, an aminoanthraquinone, and combinations thereof.

47. The particle of any one of claims 45-46, wherein the thermally activatable fragmentation group produces a nucleophilic group upon activation.

48. The particle of any one of claims 45-47, wherein the thermally activatable fragmentation group comprises substituted and unsubstituted carbonates, carbamates, esters, lactams, lactones, amides, imides, oximes, sulfonates, or phosphonates.

49. The particle of any one of claims 39-48, wherein the particle is amorphous, partially amorphous, or partially crystalline.

50. The particle of any one of claims 39-49, wherein the particle further comprises a shell enclosing the particle to form a core-shell particle

51. The particle of claim 50, wherein the shell comprises a cross-linked polymer.

52. The particle of claim 50, wherein the shell comprises an organosilicate polymer derived from vinyl trimethoxysilane reagent in Stöber synthesis.

53. The particle of claim 39, wherein the carrier is crosslinked.

54. The particle of claim 1, wherein the color is changed to colorless.

55. The particle of claim 1, wherein the color is changed from one hue to a different hue.

56. The method of claim 35, wherein the laser pulse duration is selected from the group of about 10 ns; about 400 ps to about 500 ps; about 500 ps to about 600 ps, and about 600 ps to about 750 ps.

57. The method of claim 35, wherein the laser emits light at 1064 nm.

58. The particle of any one of claims 36-38, wherein the material for absorbing the radiation at infrared wavelengths is Epolight IR 1117.

59. The particle of any one of claims 33-38, wherein the colorant is one or more selected from the group of magenta, cyan, yellow, black and PB5 disclosed in the Table 1.

60. The particle of any one of claims 33-38, wherein the colorant is one or more selected from the group of magenta, cyan, yellow, black and PB5 disclosed in the Table 3.
